(19) United States
(12) Patent Application Publication
(10) Pub. No.: US 2009/0105091 A1
(43) Pub. Date: Apr. 23, 2009

(54) Modified Amino Acids

(75) Inventors: Ulrich Jordis, Vienna (AT); Jaywant B. Phopase, Vienna (AT)

Correspondence Address: FULBRIGHT & JAWORSKI L.L.P., 600 CONGRESS AVE., SUITE 2400, AUSTIN, TX, 78701, US

(73) Assignee: TECHNISCHE UNIVERSITAT WIEN, Vienna (AT)

(21) Appl. No.: 12/280,919

(22) PCT Filed: Nov. 24, 2006
(86) PCT No.: PCT/EP06/11264

§ 371 Date: Aug. 27, 2008

(30) Foreign Application Priority Data
Feb. 27, 2006 (EP) 06450025.9

(51) Int. Cl. C40B 40/04; C07C 229/02; C07C 271/06; C07D 209/04; C07D 207/02; C07K 5/10; C07K 7/06

(52) U.S. Cl. 506/15; 560/38; 560/34; 560/24; 548/494; 548/533; 540/460; 560/169; 530/330; 530/329; 530/328; 530/333

(57) ABSTRACT

The present invention provides a compound of the general formula (I), wherein X is the connection between the CO-hydrazine and the NR¹-oxalic acid or ester group, and uses and synthesis methods. These compounds represent amino acid derivatives, wherein the amine group is turned into an acidic group by the oxalic acid group and the carboxylic acid is turned into an amine functionality by the hydrazine group; as well as peptidomimetics comprising the compound and methods for their synthesis.

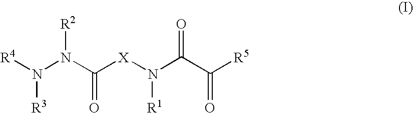

The present invention relates to modified amino acids and their use as building blocks for peptide and amino acid mimetics or analogs.

Amino acid derivatives are used or have been used as chemical moieties which mimic the biological function of an amino acid with modified stability, degradation or reactivity properties, especially in peptide mimics such modified amino acids or amino acids analogs are used. For example, amino acid derivatives have applications in the synthesis and manufacture of a wide range of pharmaceutical products and therapeutic agents used for the treatment of human or animal diseases or crop protecting agents such as herbicides, insecticides or fungicides.

One particular aspect in the design of modified peptides is the goal to obtain products that are metabolically stable and still exert their desired properties by providing a three dimensional arrangement of their natural or unnatural amino acid residues for interaction with and/or binding to their biological targets like receptors, enzymes, proteins and other macro- or small molecules. As an example the secondary structure peptidomimetical approach is a rational way to develop novel nonpeptide pharmaceutical agents based upon biologically significant proteinaceous leads (Eguchi et al., Mini-Reviews in Medicinal Chemistry (2002), 2(5): 447-462). Amino acid derivatives include ketones, aldehydes, acetals, esters, ethers, etc. and are designed for increased stability to prolong bioavailability of e.g. a peptide pharmaceutical or to increase its reactivity with a specific target for the targets inhibition.

In some areas lead compounds are derived by testing libraries of natural or unnatural peptides. As typically such leads cannot be used as drugs or other bioactive substances as they are either metabolically unstable or exert other unfavourable physicochemical properties, such leads are then altered in various ways, e.g. by preparing retro, inverso-, and retro-inverso analogs, by introducing additional conformational fixations, insertions or deletions of the original peptide motif, the compounds of the present invention can be used either as building blocks and sub-structures to arrive at biological active moieties or are biologically active themselves (WO 94/05311 A1). E.g. Ranganathan et al. (J. Chem. Soc. (1) (1993):92-4) describe oxal amides as retro-peptido mimetica.

According to the U.S. Pat. No. 5,618,914 B a peptide mimetic is disclosed forming a beta turn motif. Therein modular components or building blocks for the synthesis of the mimetic are provided, which can be assembled to a variety of three dimensionally constrained beta turn motifs. These building blocks are amino acid derivatives, wherein a linker group is ligated to the amino group of the template amino acid.

A peptidomimetic is a small protein-like chain, ring or ring/chain combination that contains both natural and nonnatural amino acids. It is designed and synthesized with the purpose of binding to target proteins in order to induce virous biological effects thus mimicking key interactions in the cell.

One example of such an effect is to induce cancer cells into a form of programmed cell death called apoptosis. All healthy cells in multi-celled organisms are subject to programmed cell death when they are no longer wanted; but cancer cells have the ability to evade apoptosis and the body's attempts to get rid of them. So peptidomimetics are part of the wide effort by researchers, research labs and institutions to create cures for cancer by means of restoring or activating apoptotic pathways in specific cells.

In the WO 2005/103012 A a hydrazino-substituted heterocyclic nitrile amino acid derivative is disclosed, among many other substances, which functions as cysteine protease inhibitor.

Methods for the synthesis of aromatic hydrazines are disclosed in the DE 1150391. These compounds are used as UV absorbents in dyes.

Compounds that mimic the structures and hydrogen-bonding patterns of protein β-sheets, but have not specifically been targeted toward binding proteins, have been reported. Kemp and co-workers described a 2,8-diaminoepindolidione molecular template that mimics the hydrogen-bonding functionality of one edge of a peptide β-strand and have coupled this β-strand mimic to peptides to generate intramolecularly hydrogen-bonded β-sheet like structures (Kemp et al. J. Org. Chem. (1990) 55: 4650-4657).

A beta sheet mimetic is disclosed in the WO 01/14412 and in an article of Nowick et al. (J. Am. Chem. Sec. (2000) 122:7654-7661). Therein the C-alpha atom is replaced by a 5-amino-2-methoxybenzoic acid, wherein the methoxy group forms a hydrogen bond with the amide group thus forming a rigid structure, which imposes the three dimensional structure of flat beta sheet motif on the peptide. This amino acid analogue has been investigated in molecular dynamics simulations to study dimerisation and beta-sheet folding mechanisms (Yu et al., Proteins: Structure, Function, and Bioinformatics (2004) 54: 116-127).

The goal of the present invention is to provide new amino acid mimetics with a broad range of applications, especially as building blocks and substructures in the synthesis of modified peptides to prepare molecules with desired properties.

Therefore the present invention provides a compound of the general formula 1,

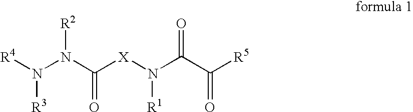

wherein
X is the connection between the CO-hydrazine and the NR¹-oxalic acid, oxalic ester or oxalic amide group and is either an unsubstituted 5-11-membered heteroaryl, or an optionally substituted group selected from C₃₋₂₀-cycloalkyl, 3-20-membered heterocyclyl, and
   linear,
   branched,
   cyclic or fused cyclic or bicyclic or fused bicyclic C₁₋₂₀-alkyl, C₂₋₂₀-alkenyl or C₂₋₂₀-alkinyl, preferably C₂₋₁₀-alkyl; and
R⁵ is selected from —SR¹⁰, —OR¹⁰ or —NR¹⁰R¹¹, provided that —NR¹⁰R¹¹ is not the amide functionality of an amino acid hydrazide, or R⁵ can cooperate with R² or R³ to form a bond or an 8 to 10 membered heterocyclic ring, and
R¹⁰ and R¹¹ are optionally substituted and independently selected from H, C₃₋₁₄-cycloalkyl, C₅₋₁₄-aryl, 3-14-membered heterocyclyl or heteroaryl, linear or branched C₁₋₁₄-alkyl, C₂₋₁₄-alkenyl, C₂₋₁₄-alkinyl and
R³ and R⁴ together may constitute a double bond to a group R¹²; wherein R¹² is optionally substituted and selected from C₃₋₁₄-cycloalkyl, 3-14-membered heterocyclyl or heteroaryl, linear or branched C₁₋₁₄-alkyl, C₂₋₁₄-alkenyl, C₂₋₁₄-alkinyl; and
R² is optionally substituted and selected from H, C₃₋₁₄-cycloalkyl, 3-14-membered heterocyclyl or heteroaryl, linear or branched C₁₋₁₄-alkyl, C₂₋₁₄-alkenyl, C₂₋₁₄-alkinyl; preferably R² is H; and
R¹, R³ and R⁴ are optionally substituted and independently selected from H, C₃₋₁₄-cycloalkyl, C₅₋₁₄-aryl, 3-14-membered heterocyclyl or heteroaryl, linear or branched C₁₋₁₄-alkyl, C₂₋₁₄-alkenyl, C₂₋₁₄-alkinyl; and
optionally at least two of R¹, R², R³, R⁴ and X can cooperate to form a (preferably monocyclic) 3 to 10 membered ring, preferably 1, 2, 3 or 4 rings are formed by R¹, R², R³, R⁴ and X, even more preferred X or in the alternative R² cooperates with one of R³ or R⁴ to form the ring; or
   an ester, amide, salt, stereoisomer or racemate therefrom;
   provided that
where X is CH₂, R³ and R⁴ are bound by single bonds and R² is selected from
   H (or D or T),
   C₃₋₂₀-cycloalkyl
   C₅₋₂₀-aryl
   3-20-membered heterocyclyl or heteroaryl
   linear or branched C₂₋₂₀-alkenyl, C₂₋₂₀-alkinyl, or unsubstituted C₁₋₄-alkyl, and
where X is heteroaryl, preferably X does not cooperate to form a ring, in particular a heterocycloalkyl ring, with R³, R⁴ or R⁵.
   The compounds of the present invention can be used as building blocks for peptide mimetic synthesis as reaction partners with natural or unnatural amino acids or as amino acid substituents in proteins. A property of the hydrazine and the oxalic acid, ester or amide is its mimicking ability of a natural amino acids amine bond.

Compounds with a hydrazine and an oxalic acid group have been described, e.g. in WO 97/22619 A2, Borloo et al. (L. Pept. Science 2(3/4) (1995):198-202), U.S. Pat. No. 4,863,947 A, Cave et al. (Europ. J. Med. Chem. 25(1) (1990):75-9), WO 2005/075475, JP 2000 141893 and WO 2005/103012, as more or less inert pharmaceutical agents or pesticides, but not as amino acid mimetics for peptide mimetic synthesis. The compounds of the invention, especially in their preferred embodiments, are designed for their reactivity with other amino acids, taking the requirements of standard a peptide synthesis, e.g. the use of protecting groups, into consideration.

In specific embodiments in the case of X and R¹ cooperating to form a cycloalkyl ring R⁵ does not comprise a further hydrazine group, R² does not form an aromatic ring with neither R³ or R⁴ and neither R³ nor R⁴ comprise sulphur. Preferably, X is part of a 3, 4, 5 or 6 membered ring in the case of X and R₁ forming a cycloalkyl ring. Especially preferred 1,6-naphthyridines or naphthyridines are excluded from the group X. These provisos can also be generalized for other compounds of formula 1 in other embodiments. Preferably in the NR¹⁰R¹¹ group R¹⁰ and R¹¹ are not covalently connected to and do not comprise another hydrazine group other than the hydrazine group of NR³R⁴NR² of formula 1. Preferably X is 3, 4, 5, 6, 7, 8, 9, 10 or 10-15 membered. Provided at least one of R³ or R⁴ forms an amide, the amide is preferably to an acidic group, preferably a protecting group. These building blocks that take the place of one or more amino acids can be used for peptidomimetics, to modify various peptides and proteins. Also comprised are pharmaceutical salts of the compound. An overview of pharmaceutical salts is given in the Handbook of Pharmaceutical Salts: Properties, Selection, and Use, P. H. Stahl and G. Wermuth (editors), publisher: Helvetica Chimica Acta, Zurich 2002.

Any aryl or heteroaryl group is preferably 5-20, more preferred 5-15 or 6 to 10, especially 6 membered, any cycloalkyl or heterocyclyl is preferably 3-20, more preferred 5-15 or 5 to 10, especially 6-8 membered, any alkyl, alkenyl or alkinyl group, optionally main chain hetero substituted, is preferably 2 to 20, more preferred 3-15 or 4 to 10, especially 5-8 membered.

The present invention provides certain novel derivatives of amino acids as shown in a compound of formula 1, wherein in an original template amino acids (exemplified by, but not restricted to, natural or unnatural, optionally substituted, alpha, beta, gamma, etc. to omega amino acids) are in carbocylic aromatic or heterocyclic compounds that are substituted by a carboxylic acid and bear an amino acid group as substituents or as part of the ring, the amino- and carboxylic acid functionalities are formally reversed in such a way that the original basic amino group is substituted by an oxalic acid function, thus rendering this end of the molecule acidic, and the originally acidic end is converted into a hydrazide, thus rendering this end of the molecule basic.

Preferably, in the compounds according to formula 1 the alkyl group is methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl. Further, the alkenyl group is preferably ethenyl, propenyl, 1-but-3-enyl, 1-pent-3-enyl or 1-hex-5-enyl. The alkynyl group is preferably ethynyl, propynyl, butynyl or pentyn-2-yl. Also preferably, the alkoxy group (or —O-alkyl group) is methoxy, ethoxy, propoxy, isopropoxy, n-butoxy, sec-butoxy, tertbutoxy, pentoxy, isopentoxy, neopentoxy, hexoxy or 3-methylpentoxy. The cycloalkyl group is preferably cyclopropyl, cyclobutyl, cyclopentyl or cyclohexyl, substituted or unsubstituted.

The aryl group is preferably phenyl, 1-naphthyl, 2-naphthyl, indanyl, indenyl, dihydronaphthyl, tetralinyl or 6,7,8,9-tetrahydro-5H-benzo[a]cycloheptenyl, substituted or unsubstituted.

The heteroaryl group is preferably pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuran-yl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide or benzothiopyranyl S,S-dioxide, substituted or unsubstituted.

In another embodiment, the heterocyclyl or heterocycloalkyl group is preferably a carbocyclic ring system of 4-, 5-, 6-, or 7-membered rings, which includes fused ring systems of 8-18 atoms containing at least one and up to four heteroatoms selected from nitrogen, oxygen, or sulfur. More preferably, the heterocycloalkyl or heterocyclyl group is morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide.

Halogen is preferably F, Cl, Br or I.

The chemical groups in a compound of formula 1 are preferably substituted by 1, 2, 3, 4, 5 or 6 substituents. The term “optionally substituted” refers to a substitution of at least one hydrogen atom of the respective chemical group, wherein the substituent is selected from the group of —OH, —SH, —NH₂, —SO₂, —SO₃, —PO₄, —O—C₁₋₄-alkyl, —S—C₁₋₈-alkyl, —NH—C₁₋₈-alkyl, —C₁₋₈-alkyl, —O—C₂₋₈-alkenyl, —S—C₂₋₈-alkenyl, —NH—C₂₋₈-alkenyl, —C₂₋₅-alkenyl, —O—C₂₋₈-alkynyl, —S—C₂₋₈-alkynyl, —NH—C₂₋₈-alkynyl, —C₂₋₈-alkynyl, —C₅₋₁₀-aryl or aryloxy, —C₅₋₁₀-hydroxyaryl, or 5 to 10 membered heteroaryl or heteroaryloxy, —C₅₋₁₀-cycloalkyl or -cycloalkenyl, 5 to 10 membered heterocycloalkyl, guanidinyl, a halogen atom, C₂₋₁₆-acyl, -acylamino or -acyloxy, amino, aminocarbonyl, alkoxycarbonylamino, azido, cyano, keto, thiocarbonyl, carboxy, carboxy-C₁₋₈-alkyl, C₅₋₁₀-arylthio, 5 to 10 membered heteroarylthio, 5 to 10 membered heterocyclyl, heterocyclylthio or heterocyclooxy, thiol, C₁₋₈-alkylthio, aminosulfonyl, aminocarbonylamino, hydroxyamino, alkoxyamino, nitro, —SO—C₁₋₈-alkyl, —SO—C₅₋₁₀-aryl, —SO—C₅₋₁₀-heteroaryl, —SO₂—C₁₋₈-alkyl, —SO₂—C₅₋₁₀-aryl and —SO₂—C₅₋₁₀-heteroaryl, carboxy, carboxy-C₁₋₈-alkyl, CF₃, substituted amino. The substituents are optionally further substituted.

Preferably R¹ or R² is hydrogen or methyl.

Heterosubstitutions provide a substitution of the main chain carbon atom by a heteroatom preferably selected from O, S, N or P. Such heterosubstitutions are included by the term “optionally substituted”. These heteroatoms are also counted for group size. Hydrogen is not counted.

If X is an aryl or heteroaryl group, X is preferably not further substituted. In the case of rigid aromatic groups the flexibility of the X group can be maintained by pre-venting further constriction through side chain substituents, whereby the compound of formula 1 can still adopt the necessary function as an universal amino acid derivative. If X is substituted aryl or heteroaryl the substituent is preferably selected from OH, O—, NH— or S—C₂₋₁₀-alkyl, C₀₋₁₀-alkyl-5-14 membered (hetero)aryl or -(hetero)cycloalkyl, SH, NH₂.

The group X in formula 1 forms the connection group between the CO-hydrazine and the NR¹-oxalic acid or ester groups. The atom numbering for the X group is performed using standard locant nomenclature for amino acids using greek letters, i.e. the alpha position is the position of the atom next to the CO-hydrazine group within X (as in amino acids the atom next to the carboxylic acid group, from which the CO-hydrazine group can be derived). The beta group would be the next main chain atom after the alpha atom and so forth. The NR¹-oxalic acid or ester group can be bound to X either on the alpha, or the beta, gamma, etc. position as in alpha, beta, gamma, etc. amino acids.

The compounds are preferably L- or D-stereoisomers at the C-alpha position.

In a specific embodiment X in formula 1 is a chemical group of one of formulas 2-6,

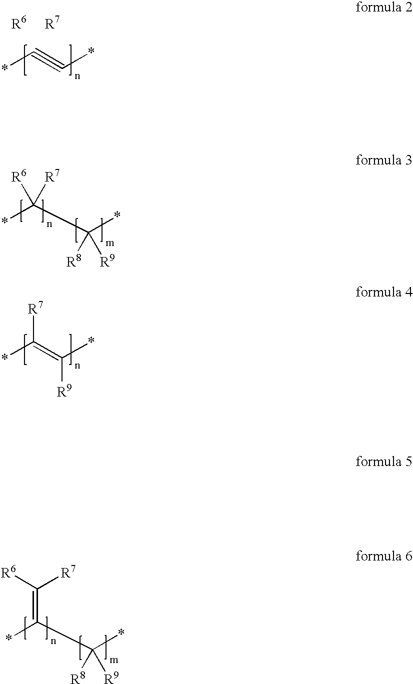

wherein R⁶, R⁷, R⁸ and R⁹ are optionally substituted and independently selected from H, C₃₋₁₄-cycloalkyl, C₅₋₁₂ aryl, 3-12-membered heterocyclyl or heteroaryl and linear or branched C₁₋₁₄-alkyl, C₂₋₁₄-alkenyl or C₂₋₁₄-alkynyl; and optionally at least two of R⁶, R⁷, R⁸ and R⁹ can cooperate to form a 3 to 22-membered (preferably 3 to 12 membered) optionally substituted or fused cycloalkyl or heterocyclic ring, or bicycles thereof; and n and m are independent integers between 0 and 5, preferably 1, 2, 3 or 4.

Preferably X is a substitute for a natural or unnatural amino acid functionality. Accordingly, side chains of these amino acids are expressed by X. In a specific embodiment X is a, preferably alpha, beta or gamma, NR¹-oxalic acid or ester bound group, selected from C₁₋₂-alkyl, guanidinylbutyl, 2-methyl-butyl, phenylethyl, p-hydroxyphenylethyl, indole-3-ylethyl, hydroxyethyl, methylthiopropyl, thioethyl, C₂₋₃-alkyl acid, C₂₋₃-alkyl acidamide, aminopentyl, 4-imidazolylethyl, or X and R¹ cooperate to form a butyl group, forming a pyrrolidine ring with the nitrogen of the NR¹-oxalic acid or ester group, wherein alpha means that the CO-hydrazine and the NR¹-oxalic acid, oxalic ester or oxalic amide group of formula 1 are bound to the same atom of the X group, preferably a carbon (“C-alpha”), beta means that the CO-hydrazine and the NR¹-oxalic acid, oxalic ester or oxalic amide group of formula 1 are bound to neighboring atoms of the X group (preferably C atoms “C-alpha” and “C-beta”, respectively) and gamma means that the CO-hydrazine and the NR¹-oxalic acid, oxalic ester or oxalic amide group of formula 1 are bound to atoms of the X group (preferably C atoms “C-alpha” and “C-gamma”) separated by one atom (preferably a C atom “C-beta”).

In especially preferred embodiments X is —CHR¹³—, wherein R¹³ is either H or D or the side chain of an amino acid selected from alanine, arginine, asparagine, aspartic acid, cysteine, glutamine, glutamic acid, glycine, histidine, isoleucine, leucine, lysine, methionine, phenylalanine, proline, 4-hydroxyproline, serine, threonine, tryptophan, tyrosine and valine, preferably the L-enantiomer therefrom.

The terms “hydrogen” or “H” also includes different isotopes of hydrogen such as H, D (i.e. ²H) or T (i.e. ³H). Other elements such as C, N, O, S, or P also include all known isotopes, stable or unstable, of these elements.

Preferably R¹⁰ and R¹¹ are selected from H, unsubstituted C₁₋₅-alkyl, C₂₋₁₀-alkenyl, C₂₋₁₀-alkinyl, C₃₋₁₄-cycloalkyl, C₅₋₁₄-aryl, 3-14-membered heterocyclyl or 3-14-membered heteroaryl.

In another embodiment X is selected from O-, S-, N- or P-heterosubstituted 3-20-membered O-, S-, N- or P-heterosubstituted heteroaryl or from the group of optionally substituted heterocycloalkyl, and linear or branched 1-20-membered heteroalkyl, 2-20-membered heteroalkenyl or heteroalkinyl, in particular selected from a O—, S—, N- or P-heterosubstituted 3-20-membered heterocycle excluding 1,6-naphthyridine.

In any way preferably one of R³ or R⁴ is a protecting group, preferably Fmoc.

Especially preferred are the compound according to formula 1 as described above, selected from the compounds of the following table 1. These compounds can be in form of a kit with 1, at least 2, at least 3 or at least 4 different compounds of table 1. The compounds are as depicted with a Fmoc protecting group also comprised by the present invention with other protecting groups on their hydrazine or oxalic functionality as depicted in table 1. However, Fmoc is the preferred protecting group.

TABLE 1
Compounds

| | |
|---|---|
| 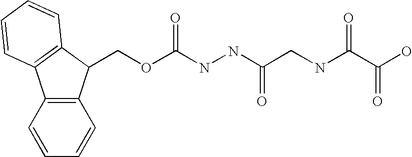 | C₁₉H₁₇N₃O₆ |
| 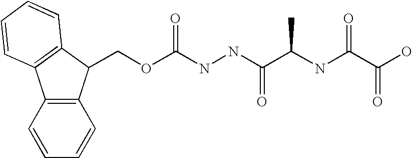 | C₂₀H₁₉N₃O₆ |
| 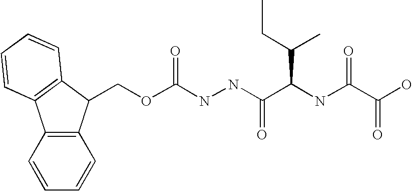 | C₂₃H₂₅N₃O₆ |
| 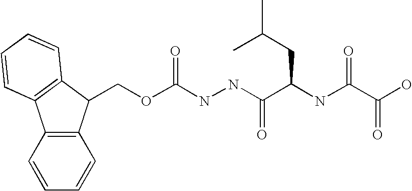 | C₂₃H₂₅N₃O₆ |
| 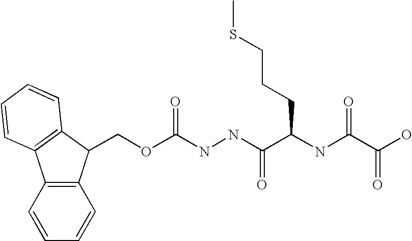 | C₂₃H₂₅N₃O₆S |
| 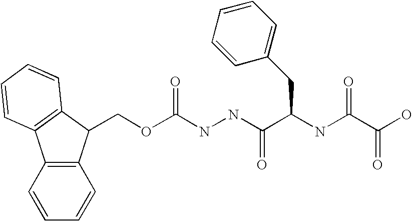 | C₂₆H₂₃N₃O₆ |
| 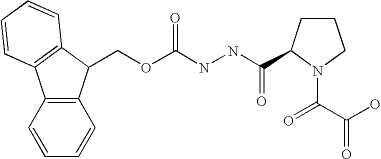 | C₂₂H₂₁N₃O₆ |
| 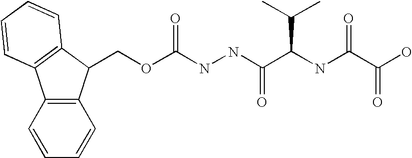 | C₂₂H₂₃N₃O₆ |
| 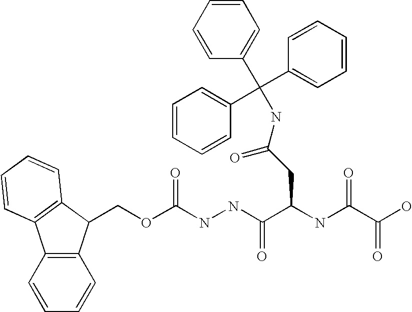 | C₄₀H₃₄N₄O₇ |
| 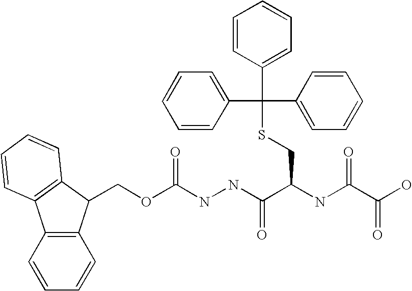 | C₃₉H₃₃N₃O₆S |
| 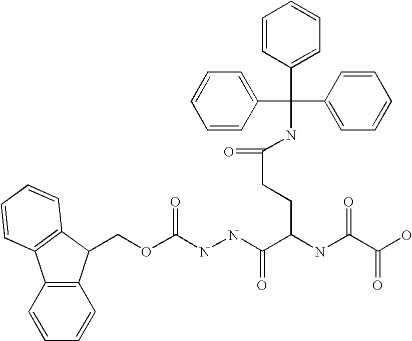 | C₄₁H₃₆N₄O₇ |
| 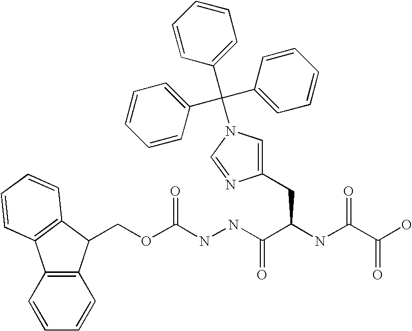 | C₄₂H₃₅N₅O₆ |
| 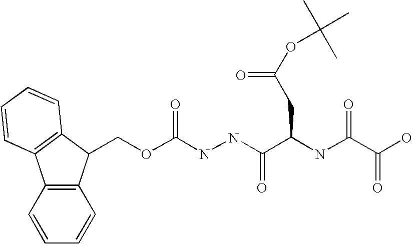 | C₂₅H₂₇N₃O₈ |
| 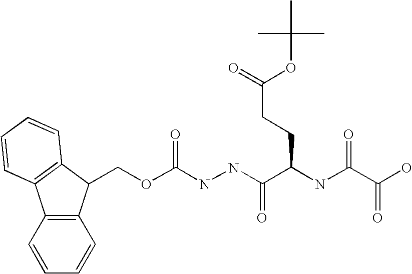 | C₂₀H₂₉N₃O₈ |
| 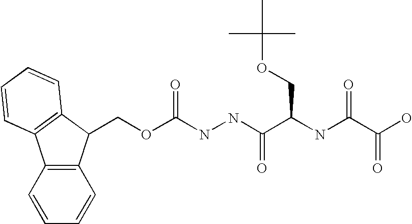 | C₂₄H₂₇N₃O₇ |
| 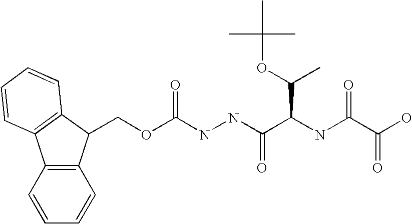 | C₂₅H₂₉N₃O₇ |
| 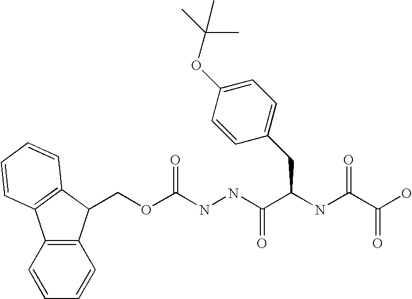 | C₃₀H₃₁N₃O₇ |
| 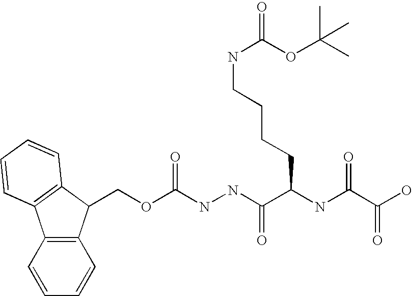 | C₂₈H₃₄N₄O₈ |
| 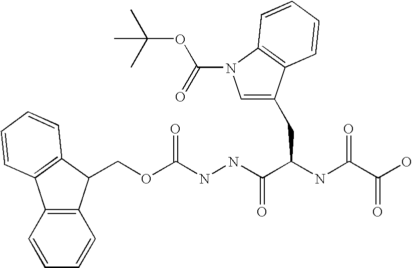 | C₃₃H₃₂N₄O₈ |
| 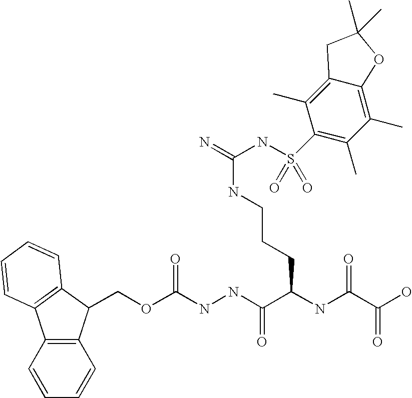 | C₃₆H₄₂N₆O₉S |
| 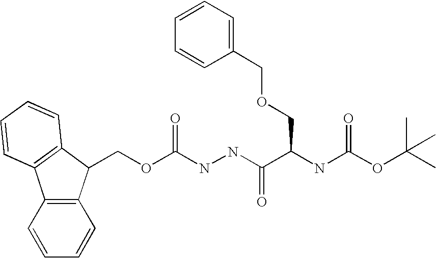 | C₃₀H₃₃N₃O₆ |
| 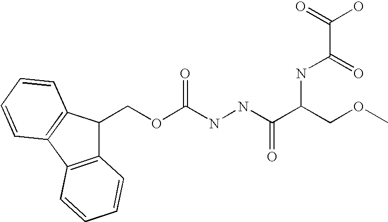 | C₂₁H₂₁N₃O₇ |
| 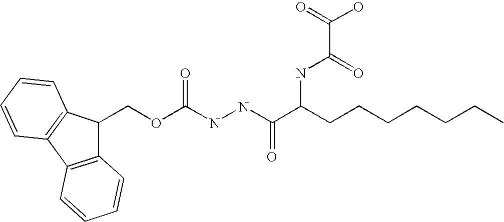 | C₂₆H₃₁N₃O₆ |
| 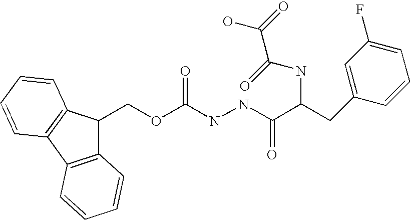 | C₂₆H₂₂FN₃O₆ |
| 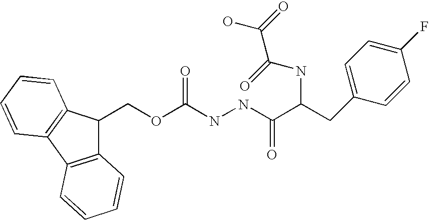 | C₂₆H₂₂FN₃O₆ |
| 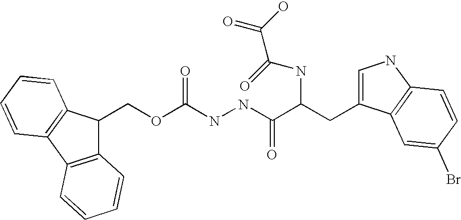 | C₂₈H₂₃BrN₄O₆ |
| 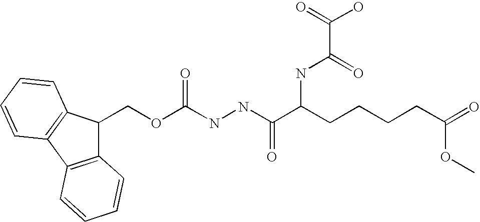 | C₂₅H₂₇N₃O₈ |
| 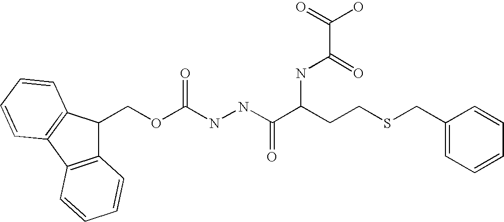 | C₂₈H₂₇N₃O₆S |
| 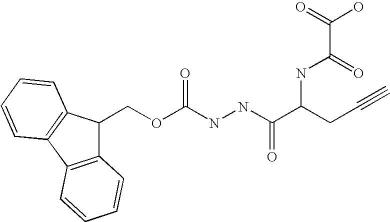 | C₂₂H₁₉N₃O₆ |
| 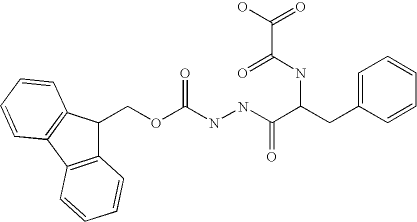 | C₄₁H₃₄N₄O₈ |
| 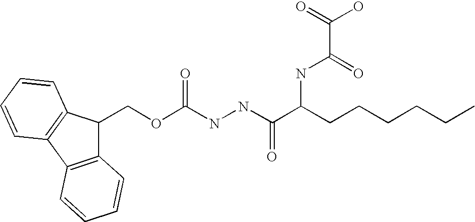 | C₂₅H₂₉N₃O₆ |
| 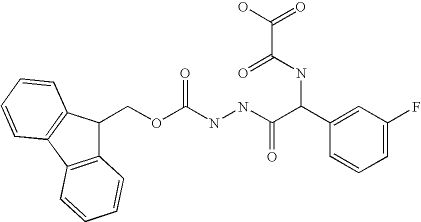 | C₂₅H₂₀FN₃O₆ |
| 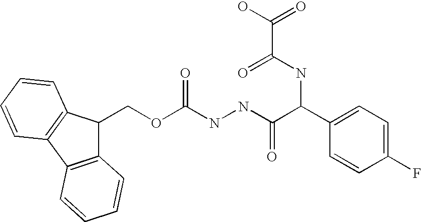 | C₂₅H₂₀FN₃O₆ |
| 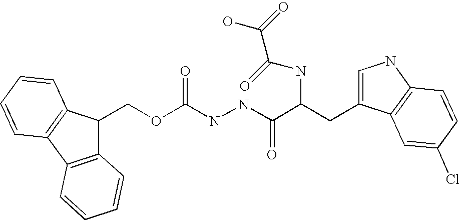 | C₂₈H₂₃ClN₄O₆ |
| 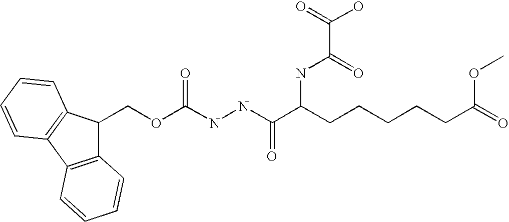 | C₂₆H₂₉N₃O₈ |
| 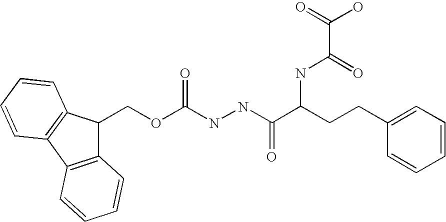 | C₂₇H₂₅N₃O₆ |
| 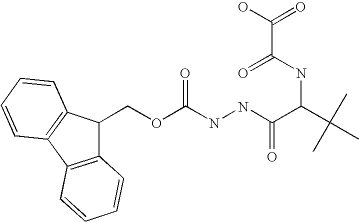 | C₂₃H₂₅N₃O₆ |
| 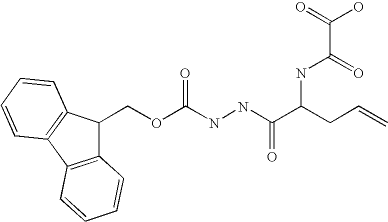 | C₂₂H₂₁N₃O₆ |
| 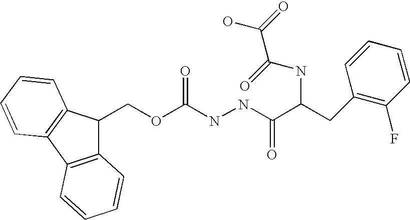 | C₂₆H₂₂FN₃O₆ |
| 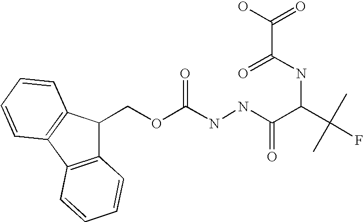 | C₂₂H₂₂FN₃O₆ |
| 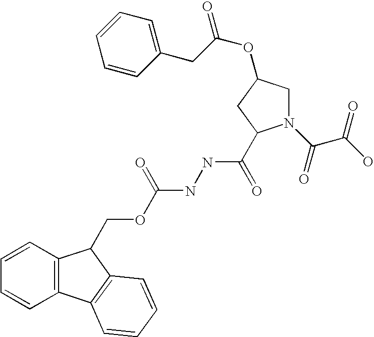 | C₃₀H₂₇N₃O₈ |
| 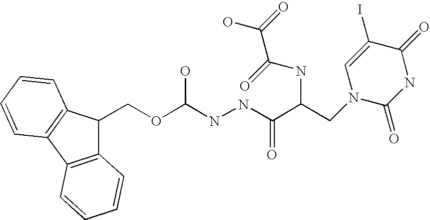 | C₂₄H₂₀IN₅O₈ |
| 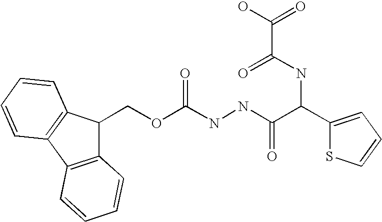 | C₂₃H₁₉N₃O₆S |
| 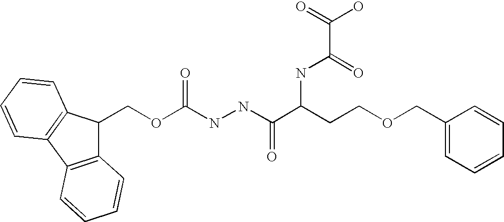 | C₂₈H₂₇N₃O₇ |
| 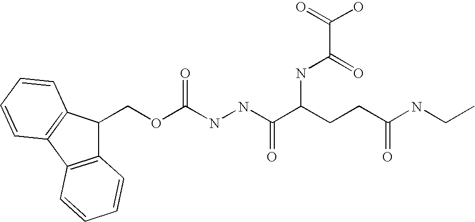 | C₂₄H₂₆N₄O₇ |
| 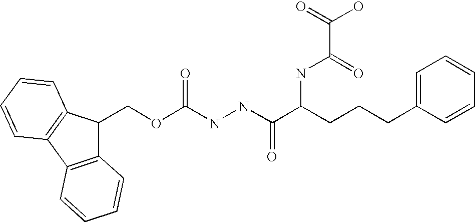 | C₂₈H₂₇N₃O₆ |
| 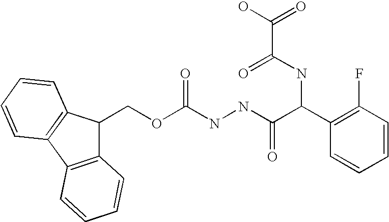 | C₂₅H₂₀FN₃O₆ |
| 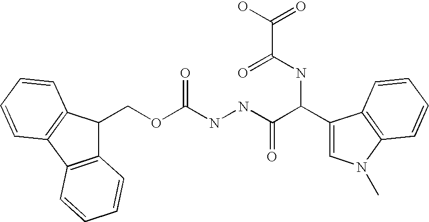 | C₂₈H₂₄N₄O₆ |
| 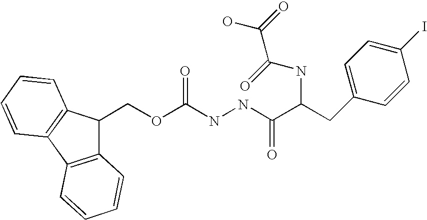 | C₂₆H₂₂IN₃O₆ |
| 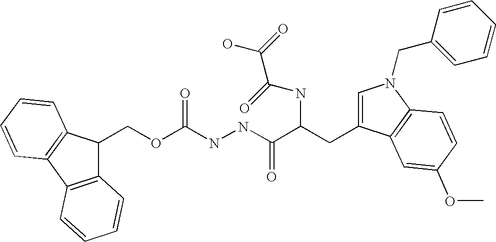 | C₃₆H₃₂N₄O₇ |
| 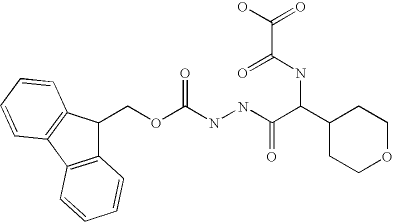 | C₂₄H₂₅N₃O₇ |
| 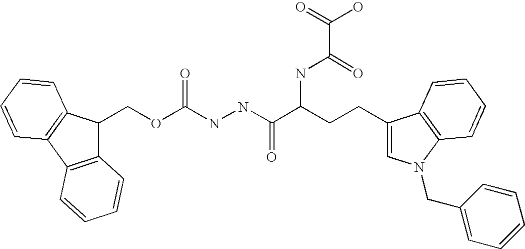 | C₃₆H₃₂N₄O₆ |
| 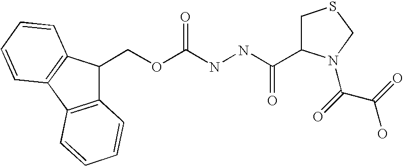 | C₂₁H₁₉N₃O₆S |
| 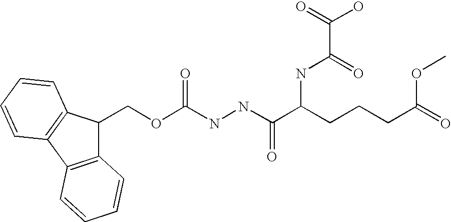 | C₂₄H₂₅N₃O₈ |
| 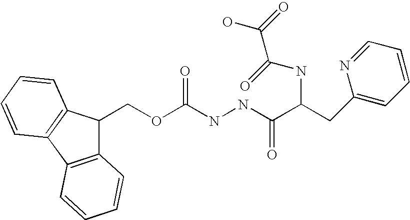 | C₂₅H₂₂N₄O₆ |
| 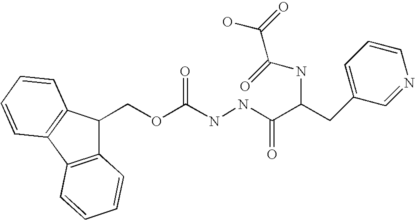 | C₂₅H₂₂N₄O₆ |
| 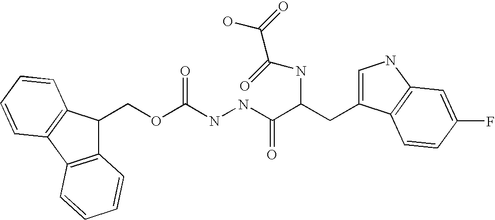 | C₂₈H₂₃FN₄O₆ |
| 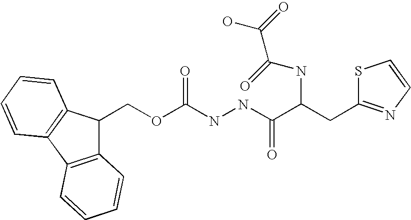 | C₂₃H₂₀N₄O₆S |
| 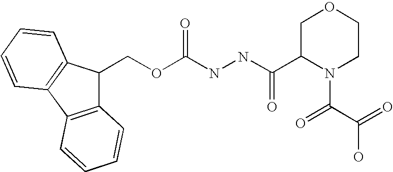 | C₂₂H₂₁N₃O₇ |
| 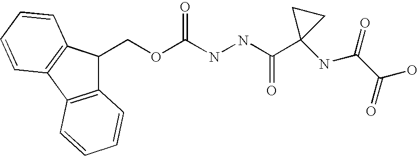 | C₂₁H₁₉N₃O₆ |
| 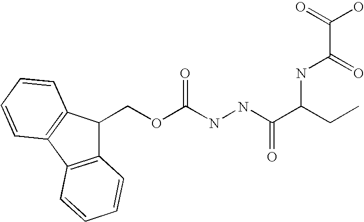 | C₂₁H₂₁N₃O₆ |
| 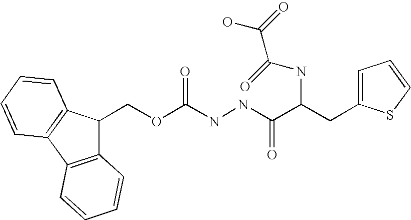 | C₂₄H₂₁N₃O₆S |
| 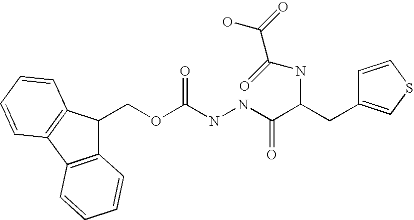 | C₂₄H₂₁N₃O₆S |
| 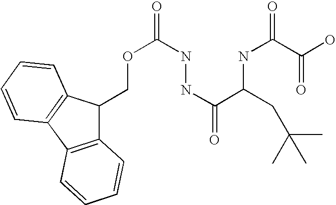 | C₂₄H₂₇N₃O₆ |
| 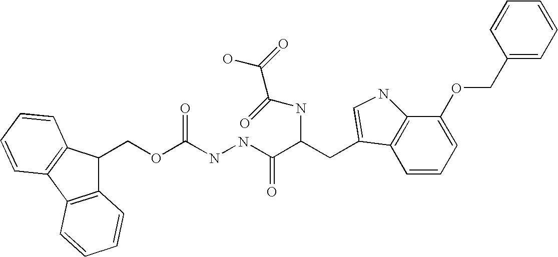 | C₃₅H₃₀N₄O₇ |
| 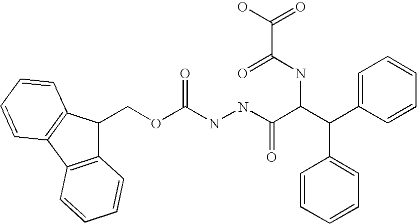 | C₃₂H₂₇N₃O₆ |
| 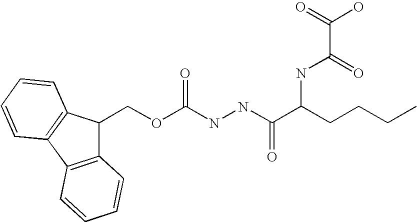 | C₂₃H₂₅N₃O₆ |
| 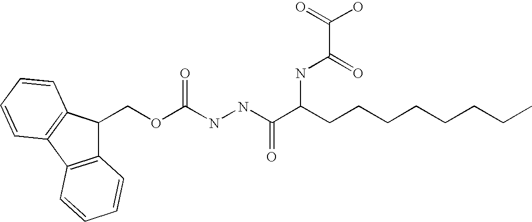 | C₂₇H₃₃N₃O₆ |
| 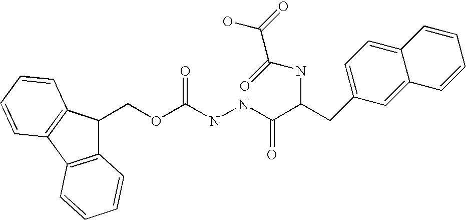 | C₃₀H₂₅N₃O₆ |
| 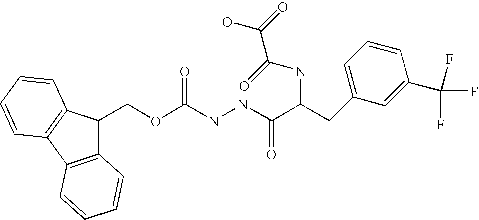 | C₂₇H₂₂F₃N₃O₆ |
| 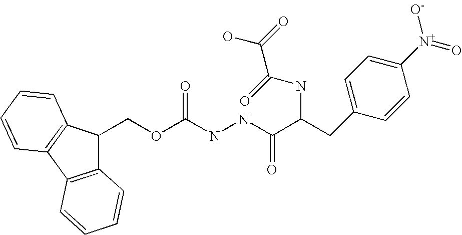 | C₂₆H₂₂N₄O₈ |
| 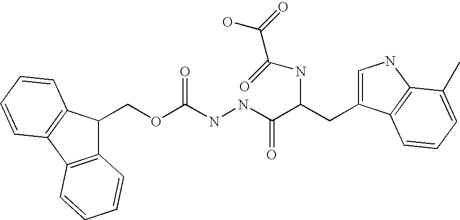 | C₂₉H₂₆N₄O₆ |
| 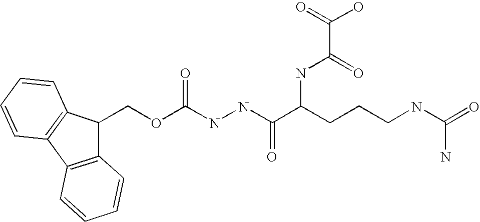 | C₂₃H₂₅N₅O₇ |
| 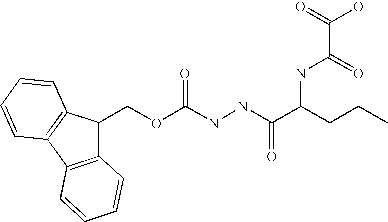 | C₂₂H₂₃N₃O₆ |
| 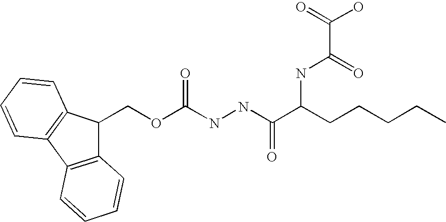 | C₂₄H₂₇N₃O₆ |
| 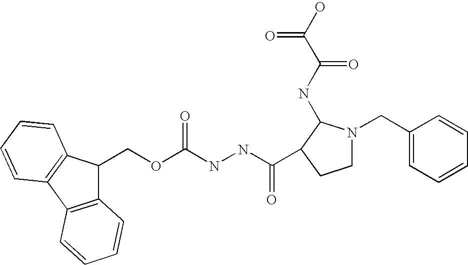 | C₂₉H₂₈N₄O₆ |
| 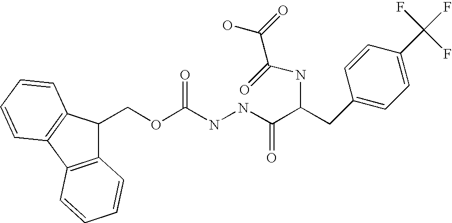 | C₂₇H₂₂F₃N₃O₆ |
| 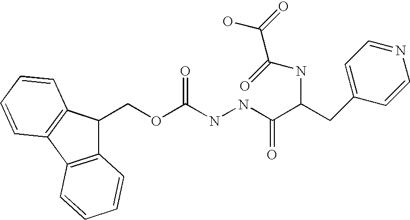 | C₂₅H₂₂N₄O₆ |
| 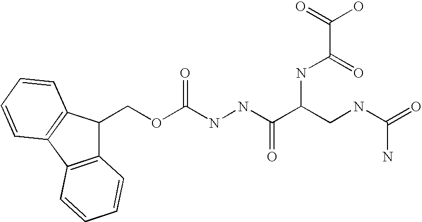 | C₂₇H₂₇N₅O₇ |
| 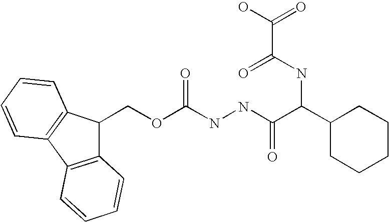 | C₂₅H₂₇N₃O₆ |
| 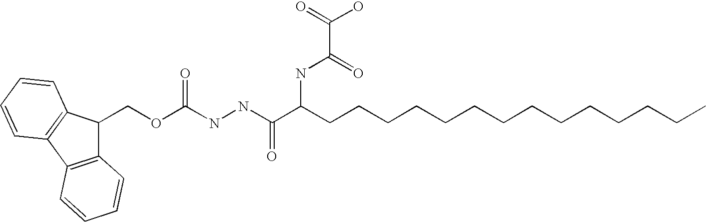 | C₃₅H₄₅N₃O₆ |
| 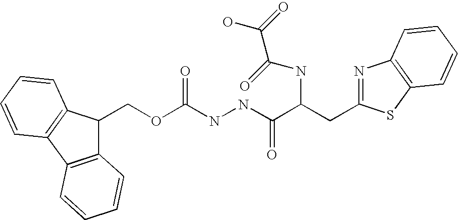 | C₂₇H₂₂N₄O₆S |
| 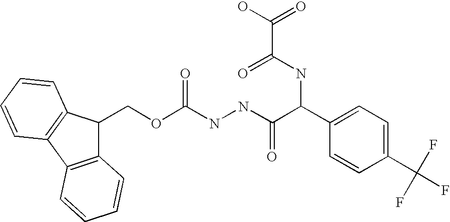 | C₂₆H₂₀F₃N₃O₆ |
| 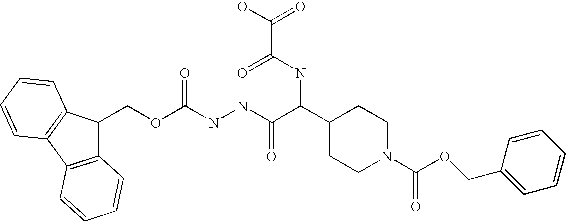 | C₃₂H₃₂N₄O₈ |
| 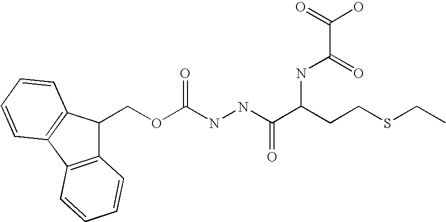 | C₂₃H₂₅N₃O₆S |
| 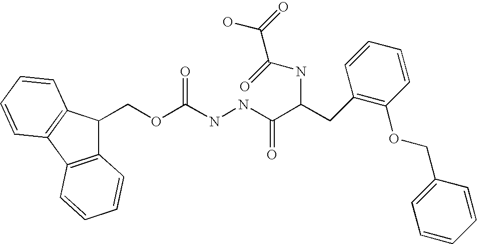 | C₃₃H₂₉N₃O₇ |
| 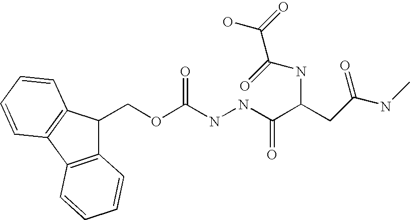 | C₂₂H₂₂N₄O₇ |
| 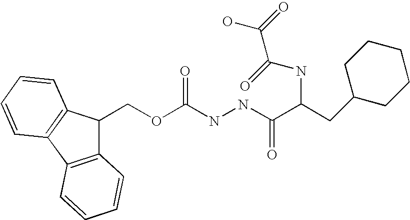 | C₂₆H₂₉N₃O₆ |
| 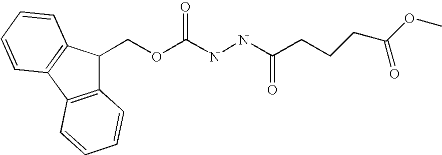 | C₂₁H₂₂N₂O₅ |
| 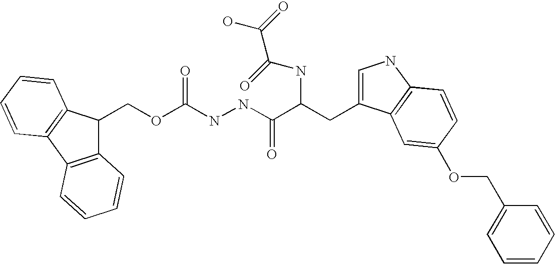 | C₃₅H₃₀N₄O₇ |
| 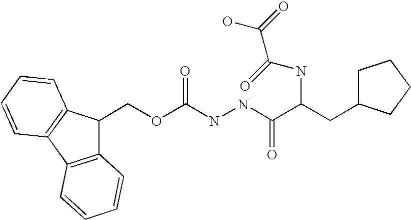 | C₂₅H₂₇N₃O₆ |
| 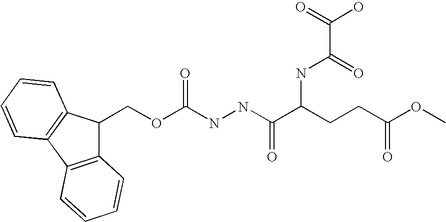 | C₂₃H₂₃N₃O₈ |
| 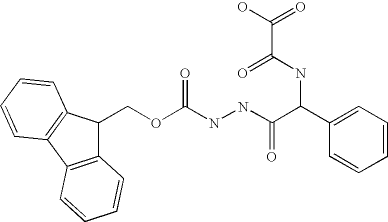 | C₂₅H₂₁N₃O₆ |

In another preferred embodiment R³ and R⁵ form a bond resulting in a heterocylic compound of the general (sub)formula 10,

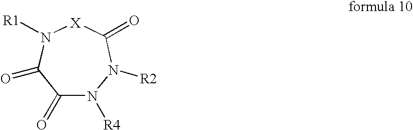

wherein X, R¹, R² and R⁴ are defined as given above. The compound of formula 10 resembles the 1,2,5-triazepine structure (especially if X is C₁-alkyl), known from e.g. Zaleska et al. (Pol. Synthesis 16 (2003): 2559-2563) and Lenman et al. (J. Chem. Soc. Perkin Trans. 1 (1997): 2297-2311). However according to the present invention all ring bonds are single bonds and the ring has an amide group and the oxalic group. As only relatively few substances of the 1,2,5-triazepine-type are known this method provides a new access to this ring system. In a further embodiment R² and R⁵ form a bond resulting in another heterocyclic compound.

In further preferred embodiments the compounds are selected from the following table 2. These compounds may also be comprised in a kit with compounds of table 1 above.

TABLE 2
Non-alpha compounds

| | |
|---|---|
| 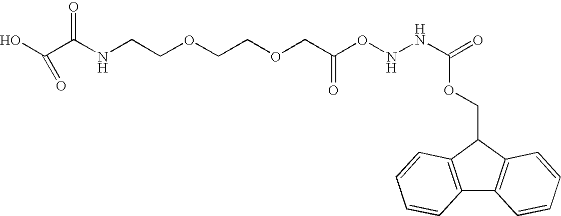 | C₂₃H₂₅N₃O₉ |
| 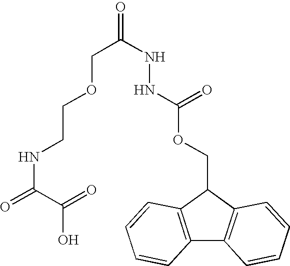 | C₂₁H₂₁N₃O₇ |
| 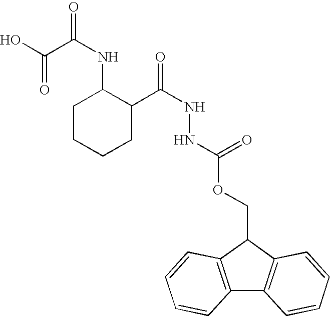 | C₂₄H₂₅N₃O₆ |
| 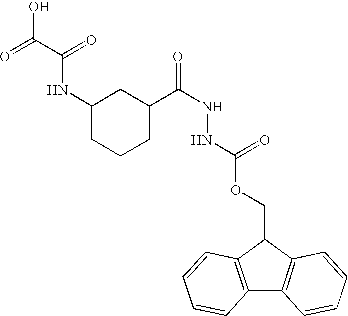 | C₂₄H₂₅N₃O₆ |
| 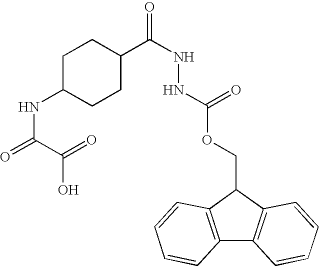 | C₂₄H₂₅N₃O₆ |
| 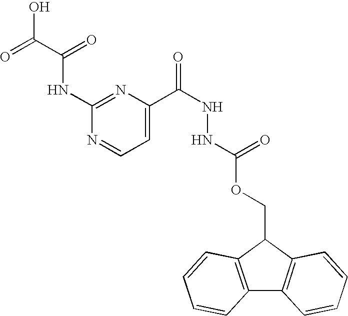 | C₂₂H₁₇N₅O₆ |
| 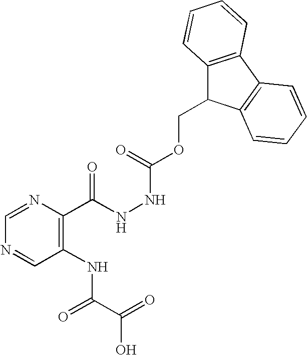 | C₂₂H₁₇N₅O₆ |
| 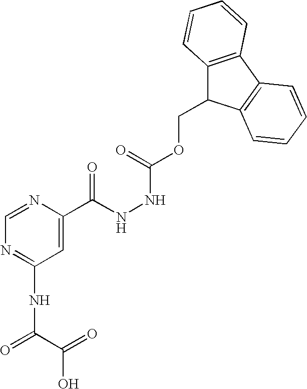 | C₂₂H₁₇N₅O₆ |
| 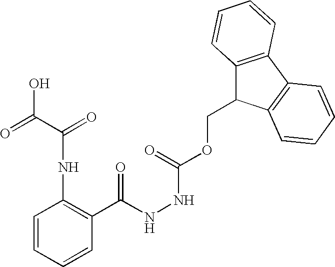 | C₂₄H₁₉N₃O₆ |
| 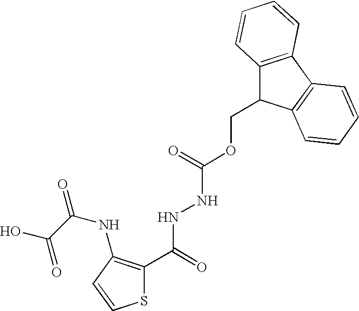 | C₂₂H₁₇N₃O₆S |
| 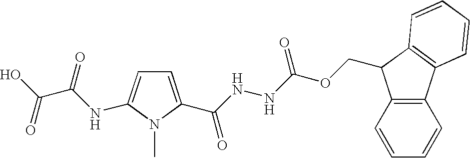 | C₂₃H₂₀N₄O₆ |
| 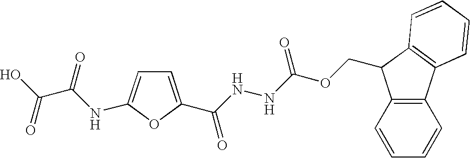 | C₂₂H₁₇N₃O₇ |

In preferred embodiments R³ or R⁴ is an N-protecting group selected from Boc, Fmoc, Alloc, trifluoroacetyl, methoxycarbonyl, ethoxycarbonyl, benzyloxycarbonyl, 2-(trimethylsilyl)ethoxycarbonyl, 2,2,2-trichloroethoxycarbonyl, arylsulfonyl, 2-trimethylsilyl)ethylsulfonyl, trityl, and/or R⁵ is a carboxylic acid protecting or activating group selected from OMe, OEt, O-t-Bu, OBn, OCHPh₂, phenacyl esters, alkoxyalkyl esters, 2,2,2-trichloroethyl esters, 2-(trimethylsilyl)ethyl esters, 2-tosylethyl esters, silyl esters or activating groups, preferably N-hydroxysuccinimid esters, 1-hydroxybenzotriazoleesters, 4-nitrophenylesters, or esters prepared in situ, preferably using the reagents HNTU (=2-(endo-5-norbornene-2,3-dicarboximido)-1,1,3,3,-tetramethyluronium hexafluorophosphate), HOCt (=1-hydroxy-1H-1,2,3-triazole-4-carboxylate, HONB (=N-hydroxy-5-norbornene-2,3-dicarboxyl, and where both the protecting or activating groups can be used alternatively in their polymer- or resin bound form.

Preferred are esters or amides of the present invention, preferably with protecting or activating groups. In a specific embodiment of the present invention the terminal amino group is substituted with a protecting group, preferably selected from Boc, Fmoc, CBz, Alloc, carbonyl, sulfonyl, sulfinyl, phosphoryl, phosphinyl. Another specific embodiment is characterized by an ester of the terminal carbonyl group in such a way, that this ester, preferably a benzyl- or t-butyl-ester, acts either as a protecting group, or as an activating group with enhanced reactivity compared to the underlying carboxylic acid. Further protecting or activating groups can be used as described in “Handbook of Reagents for Organic Synthesis: Activating and Agents and Protecting Groups. Pearson, Anthony J.; Roush, William R.; Editors. UK. (1999), 513 pp. Publisher: (Wiley, Chichester, UK) or Protective Groups in Organic Synthesis. 2nd Ed. Greene, Theodora W.; Wuts, Peter G. M. USA. (1991), 473 pp. Publisher: (John Wiley and Sons, Inc., New York, N.Y.).

A multitude of protecting groups is known in the state of the art. An example of an amine or OH protecting group is benzylcarboxycarbonyl (abbr. Z or Cbz) with the general reactivity:

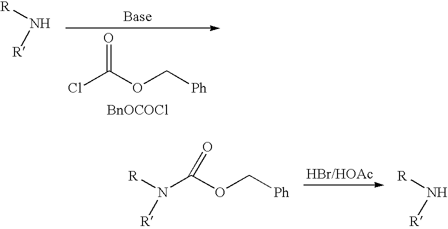

The aromatic phenyl (Ph) therein can optionally be substituted, e.g. by a halogen atom (see examples), or further varied as is known in the field of organic chemistry, e.g. by further substitution of one to three alkoxy (typically methoxy) groups allowing the deprotection steps to proceed under milder condition. Further preferred protecting groups include the t-Butoxycarbonyl (t-BOC)

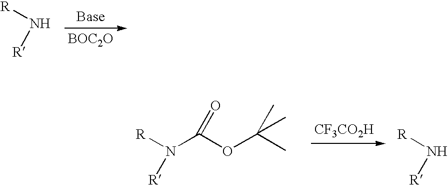

or the 9-Fluorenylmethoxycarbonyl (Fmoc) protecting group,

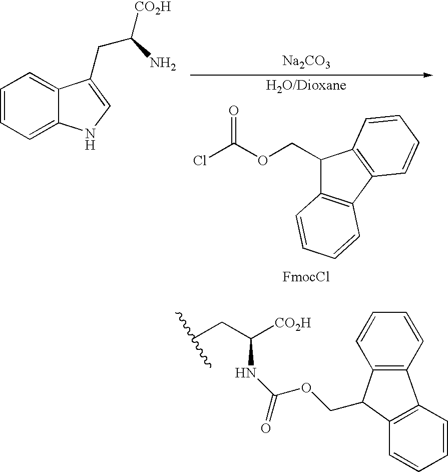

among others. Bound protecting groups in a compound according to the present invention preferably include 9-fluorenylmethyl carbamate (Fmoc-NRR′), t-butyl carbamate (Boc-NRR′), benzyl carbamate (Z-NRR′, Cbz-NRR′), acetamide, trifluoroacetamide, phthalimide, benzylamine (Bn-NRR′), triphenylmethylamine (Tr-NRR′), optionally substituted with additional chlorine atoms, benzylideneamine, p-toluenesulfonamide (Ts-NRR′), N-allyloxycarbonyl (Alloc) ester, methyl ester, t-butyl ester, benzyl ester, S-t-butyl ester, 2-Alkyl-1,3-oxazoline, acetic acid ester, pivalic acid ester, benzoic acid ester, sulfonyl ester, sulfinyl ester, phosphonyl ester, and amino acid ester. An extensive overview of protecting groups, which can be used according to the present invention is given in “Synthesis of peptides and peptidomimetics” ed. M. Goodman, Vol. 22a and 22b (protecting and activating groups), 22c and 22d (peptide/peptidemimetic synthesis), Georg Thieme Verlag, Stuttgart, New York (2002). Volumes 22c and 22d also disclose methods for side chain modification, which can also be employed when synthesizing (derivative) compounds according to formula 1 and peptidomimetics of the present invention.

In a compound according to the present invention the protecting group preferably replaces R³, R⁴, R⁵ or R¹⁰ thus forming an ester or amide of the CO-hydrazine or the NR¹-oxalic acid group thus formally replacing R³, R⁴, R⁵ or R¹⁰.

The present invention also provides a method for the manufacture of a compound of formula 1, defined above, wherein a compound or precursor according to formula 7,

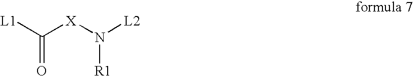

is contacted by a hydrazine or hydrazine derivative of formula 8

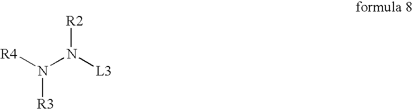

and an oxalic acid, ester or derivative of formula 9,

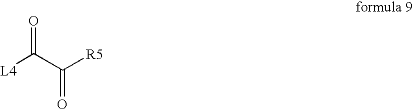

in any order, wherein X, R¹, R², R³, R⁴ and R⁵ are defined as given above for the final compound of formula 1, above, and L1, L2, L3 and L4 are independent selected arbitrary leaving groups, preferably L4 is Cl or OH or an activated ester, e.g. phenyl- or 4-nitrophenylester, or an anhydride including mixed anhydride, e.g. MeO-CO—CO—O—CO—CO-OMe or 1,4-dioxane-2,3,5,6-tetrone. Preferably the compound according to formula 9 is an oxalic acid ester chloride or oxalic acid anhydride. Further chemical bonds between two of X, R¹, R², R³, R⁴ and R⁵ can be formed by conventional chemical synthesis procedures, including ester, amide and protecting and activating group chemistry. Preferably, the compound of formula 9 can be added before or after the addition of the compound of formula 8. If all compounds are added in one step mixtures of all starting substances can result.

Leaving groups are well known in the field of organic chemistry and form preferably conjugate acids (see also “Synthesis of peptides and peptidomimetics”, above). Their functionality is characterized by an inherent instability. Preferably L1, L2, L3 and L4 are independently selected from amine (—NH₂), methoxy (CH₃O—), hydroxyl (HO—), carboxylate (CH₃COO—), —NO₃, F—, Cl—, Br—, I—, azide (N₃—), thiocyanate (SCN—), nitro (—NO₂) and cyanide (—CN). L2 and L3 are preferably hydrogen.

In a most preferred embodiment R³ or R⁴ is a protecting group, preferably Fmoc, for example synthesized by reacting Fmoc-C₁-anhydride with NH₂—NH₂, or more generally NHR³—R²L3.

The compound according to the present invention is preferably used to create a peptide mimetic with other amino acids or amino acid mimetics. Such a protein or peptide mimetic comprises the compound according to the general formula 1

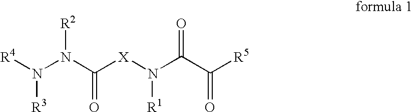

wherein X, R¹, R², R³, R⁴ and R⁵ are defined as in any one of claims 1 to 10 as a molecular part, and a natural or unnatural amino acid or an additional amino acid mimetic, preferably bound by an amide bond. Thus in its minimal the peptide or protein mimetic comprises at least two amino acids or amino acid mimetics, wherein at least one is as described by formula 1. Preferably the protein or peptide mimetic is comprised of at least three, four, five or at least six amino acids or amino acid mimetics (amino acid analoga). In particular at least 2, preferably at least 3, even more preferred at least 5, especially preferred at least 10 or most preferred at least 20, natural or unnatural amino acids or additional amino acid mimetics in addition to the compound of formula 1 are comprised by the peptide or protein mimetic.

Alternatively, in the peptide or protein mimetic X is the connection between the CO-hydrazine and the NR¹-oxalic acid or ester group and is either a bond, 5-20 membered heteroaryl or aryl, or an optionally substituted group selected from C₃₋₂₀-cycloalkyl, 3-20 membered heterocyclyl, and linear or branched C₁₋₂₀-alkyl, C₂₋₂₀-alkenyl or C₂₋₂₀-alkinyl; and

R⁵ is selected from —OR¹⁰ and —NR¹⁰R¹¹, or R⁵ can cooperate with R² or R³ to form a bond or a 8 to 10 membered heterocyclic ring; and
R³ and R⁴ together may constitute a double bond to a group R¹²;
R¹, R², R³, R⁴, R¹⁰, R¹¹ and R¹² are optionally substituted and independently selected from H, C₃₋₁₄-cycloalkyl, C₅₋₁₄-aryl, 3-14 membered heterocyclyl or heteroaryl, linear or branched C₁₋₁₄-alkyl, C₂₋₁₄-alkenyl, C₂₋₁₄-alkinyl; and optionally at least two of R¹, R², R³, R⁴ and X can cooperate to form a 3 to 12 membered cycloalkyl or heterocycloalkyl ring; or
an ester, amide, salt, stereoisomer or racemate therefrom;
as a molecular part, and a natural or unnatural amino acid or an additional amino acid mimetic, preferably bound by an amide bond. As in the case of the sole compound according to formula 1, in the case of X being aryl or heteroaryl X is preferably not substituted. The compound of formula 1 is most preferably linked to another main amino acid via its hydrazine or oxalic acid groups, preferably by both to different amino acids. The compound of formula 1 is therefore comprised in a peptide mimetic among other amino acids, peptides, or other amino acid analoga, preferably comprising 1, 2, 3, 4, 5, 6, 7, 8, 9 or 10 further natural or unnatural amino acids. Preferably at least one of these amino acids is selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or proline, most preferred connected at the R³, R⁴ or R⁵ position, replacing any possible substituents at these positions in the compound of formula 1. Another preferred additional compound or peptide mimetic substructure is phenyl ethylenediamine, which can be used to generate turn motifs. Such an amide can mimic the three dimension appearance of an amino acid, e.g. the hydrogen bonding acceptor and donor properties of the amide bond, found in proteins. Amazingly, with the compound of formula 1 most native protein structures can be created, including alpha-helices, and beta-sheets.

Therefore, in a preferred embodiment the peptide mimetic mimics the three dimensional structure of an alpha-helix, beta-sheet or turn motif. Through this three dimensional structure native protein motifs can be formed, which allows the use of the compound in substantial protein or peptide parts. It is thus possible to form peptide mimetics with the biological functionality of the respective templates, but with modified stability, bioavailability or distribution as examples of its modified chemical properties.

Interactions between the hydrogen-bonding edges of beta sheets occur widely in protein quaternary structure, protein-protein interactions, and protein aggregation and are involved in both healthy biological processes and in diseases ranging from cancer and AIDS to anthrax and Alzheimer's. These protein-protein interactions constitute a form of molecular recognition of great importance in biological processes and because of its fundamental nature. The compounds of the present invention can be used to synthesize modified peptides that recognize protein beta-sheets in a sequence-selective fashion. A database for beta-sheets, wherein the compounds of the present invention can be incorporated is established in the internet (http://www.igb.uci.edu/servers/icbs/).

Protein secondary structures such as α-helices, β-sheets, and β-turns are important features of the three-dimensional structure and biological activity of proteins. The mimicry of peptide and protein structures has emerged as a focal point of bioorganic and medicinal chemistry. Designing secondary structure mimics composed of short peptides has attracted much attention in the development of pharmacologically active compounds, artificial receptors, asymmetric catalyst and new materials. A substantive examination of artificial alpha-helices, beta-sheets and turns, as well as their design was reviewed by Rizo and Gierasch (Annu. Rev. Biochem 1992. 61:387-418). The chemical synthesis of such a mimetic can be performed as described herein, e.g. by solid phase synthesis using a compound of formula 1. The prognosis of a fold for alpha-helices, beta-sheets and (beta-)turns can be designed according to Rizo and Gierasch, or Loughlin et al. (Chem Rev 2004, 104:6085-6117).

Compounds that mimic the structure and hydrogen-bonding patterns of protein β-sheets are of interest as drug candidates and as model systems with which to study protein structure and stability. β-sheet formation plays a critical role in many biological processes associated with diseases and normal functions. β-sheet interactions between proteins have been shown or hypothesized to be involved in cell signalling and oncogene expression associated with the binding of Ras and Rap by the serine/threonine kinase Raf, the clustering of membrane ion channels by PDZ domains, the binding of lymphocyte function-associated antigen-1 (LFA-1), by the intercellular adhesion molecule-1 (ICAM-1), and the interaction between the CD4 receptor and the HIV viral protein gp120, to cleave peptides, proteolytic enzymes, such as HIV-1 proteases or renin, form β-sheet-like networks of hydrogen bonds with their peptide substrates. HIV-1 protease dimerizes through four-interchelating β-strands. The met repressor, a protein involved in gene regulation, also functions as a β-sheet dimer; in this case, the β-sheet that forms is directly involved in binding to the major groove of DNA. Many proteins aggregate to form insoluble β-sheet structures that are associated with Alzheimer's disease, Kreutzfeld-Jacob disease and other prion diseases, and progressive neurodegenerative disorders that are associated with trinucleotide (CAG) repeats.

The compounds of the current invention can provide alternating arrays of hydrogen-bond donors and acceptor patterns like a tripeptide β-strand and can be used as β-strand mimics to form β-sheet-like structures in combination with an appropriate molecular scaffold. The application of a compound according to formula one in a beta-sheet is realized by hydrogen bond donor and acceptor functionalities, for example according to the following scheme:

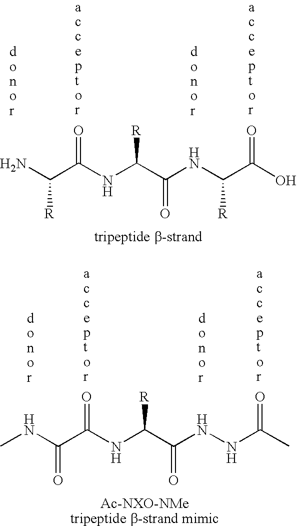

2D and 3D structures of a specific example of the compound in a β-strand are given in FIG. 1.

In the 20 proteinic amino acids, proline is the only amino acid whose Ca—N bond is a part of the pyrrolidine ring. This cyclic side chain imposes strong restrains on peptide conformation. Proline if quite often observed at the (i+1) position of β-turn structure. The proline derivatives (wherein X cooperates with R¹ to form a ring structure) can also be used as beta turn mimics, as an example FIG. 3 is a structure of a turn-motif.

A compound of formula 1 can e.g. be derived from a natural amino acid, wherein the amino functionality of the amino acid was transformed into a carboxylic functionality through the oxalic acid ligation, and the carboxylic acid functionality was transformed into an amidic functionality by the hydrazine. These reversed functionalities can, in analogy to the amine and carboxylic functionalities of an amino acid, be used for the same purposes, like liquid or solid phase protein synthesis using the same or similar protective groups developed for amino acids.

The present invention provides a method for the synthesis of a peptide mimetic using a compound of the general formula 1, wherein a reaction target comprising an amine is contacted with the compound according to formula 1, and R³ or R⁴ constitutes an amide with a protecting group, preferably Fmoc. Through this step a peptide bond is formed under standard conditions for peptide synthesis. In this step of common peptide synthesis procedures the amino acid to be bound is replaced by the compound according to formula 1. The amino group containing reaction target can be an amino acid or a derivative or another target, like a solid resin. The Fmoc and Boc protecting groups are especially preferred, since they can again be removed by an irreversible process, wherein the carboxylic group of Fmoc or Boc is removed through CO₂ elimination, preferably under acidic conditions by trifluoroacetic acid. This has the advantage of high yield, especially in combination with solid phase synthesis, wherein a peptide is stepwise synthesized onto a solid resin. Contrary to natural biosynthesis at the ribosome, solid-phase peptide synthesis preferably proceeds in a C-terminal to N-terminal fashion, wherein the amino group is protected by an arbitrary protecting group. In a next step this protecting group is removed, e.g. the amide with the R³ or R⁴ protecting group of the compound according to formula 1. Amino acid chain elongation, or amino acid mimetic ligation, can then proceed by binding the carboxylic functionality of the next amino acid or an amino acid mimetic to the now unprotected amino functionality. The compound according to the present invention is an example of such an amino acid mimetic, among others known in the state of the art.

In a further aspect of the method provided herein an amino acid or amino acid mimetic, preferably with a protected amino group is contacted with the compound according to formula 1, preferably after the step of removing the R³ or R⁴ protecting group.

Preferably, the method for peptide mimetic synthesis is a solid phase synthesis method, i.e. a reaction target is solid, preferably a solid resin, e.g. the Merrifield resin, a copolymer of styrene and chloromethylstyrene in bead form (Steward and Young, Solid Phase Peptides Synthesis, Pierce, Rockford, Ill. (1984)). This includes cases where amino acids or other chemical compounds have already been ligated to the resin and the compound of formula 1 is ligated to the amine groups of these immobilized amino acids or compounds. Of course, further amino acids and other chemical compounds can be ligated to the compound of formula 1 afterwards.

In the examples several solid phase synthesis methods have been applied with the incorporation of building blocks of formula 1 into artificial peptides. These building blocks can also be used by peptide synthesis robots. The term “protecting groups” also comprises the solid phase versions of protecting groups. As these building blocks can be integrated into solid phase synthesis and automated peptide synthesizers, there will be numerous applications of the compounds of formula 1.

In another aspect the present invention provides a protein or protein fragment comprising the compound of the general formula 1 as covalently bound insert at any position of the protein amino acid sequence. To this end the protein can be synthesized using artificial, biological or microbiological methods. The compound of formula 1 can be attached to such a protein or protein fragment or two fragments, for example two fragments of one protein, whereby the compound of formula 1 is inserted into the amino acid sequence of the protein.

To this end a method for the manufacture of a protein or protein fragment is provided, comprising the step of ligating a compound of formula 1 to a peptide, protein or protein fragment. As mentioned above, the advantage of a compound of formula 1 lies in the usability of standard protein and peptide synthesis methods.

Preferably, the method further comprises the step of ligating a further peptide, protein or protein fragment to the compound of formula 1. Preferred is the use of the compound of formula 1 as amino acid substitute (wherein one or more amino acids of the protein is/are substituted) in a protein, e.g. an affinity peptide, wherein the compound stabilizes the peptide in a specific three dimensional structure.

A preferred use for the compound according to the invention is as a spacer moiety, especially as a hydrophilic spacer. Spacer moieties have a wide range of application. For example in biochemistry spacers are used for the immobilization of pharmacologic agents on biomolecules, e.g. antibodies, or for the immobilization of affinity targets on a resin for the purposes of chromatography. The compounds disclosed herein have the amino and carboxylic features of a natural amino acid, and can be handled by standard protein chemistry techniques. Therefore, the compounds of formula 1 can be used as linker moieties, wherein the length of the linker can be determined by the size of the X group. Especially in the field of bioaffinity purification such a linker is excelled by low unspecific binding of unwanted biomolecules.

In another aspect the present invention provides the use of a compound according to the present invention as components in dynamic combinatorial libraries. Dynamic combinatorial libraries consist of library member is assembled from building blocks that are connected through reversible bonds for dynamic combinatorial chemistry. Dynamic combinatorial chemistry is an approach that uses self-assembly processes to generate libraries of chemical compounds for e.g. host-guest interactions and drug discovery. The compounds of the present invention can be used as such building blocks and the amino acid, protein or peptide mimetics as library members.

The present invention is further illustrated by the following figures and examples, without being limited thereto.

### FIGURES

FIG. 1: A: 2D structure of a beta-sheet model comprising a modified Alanin (X in formula 1 is a C₁-alkylated C-alpha), lower strand. B: 3D-structure of a beta-sheet stabilized by hydrogen bonds.

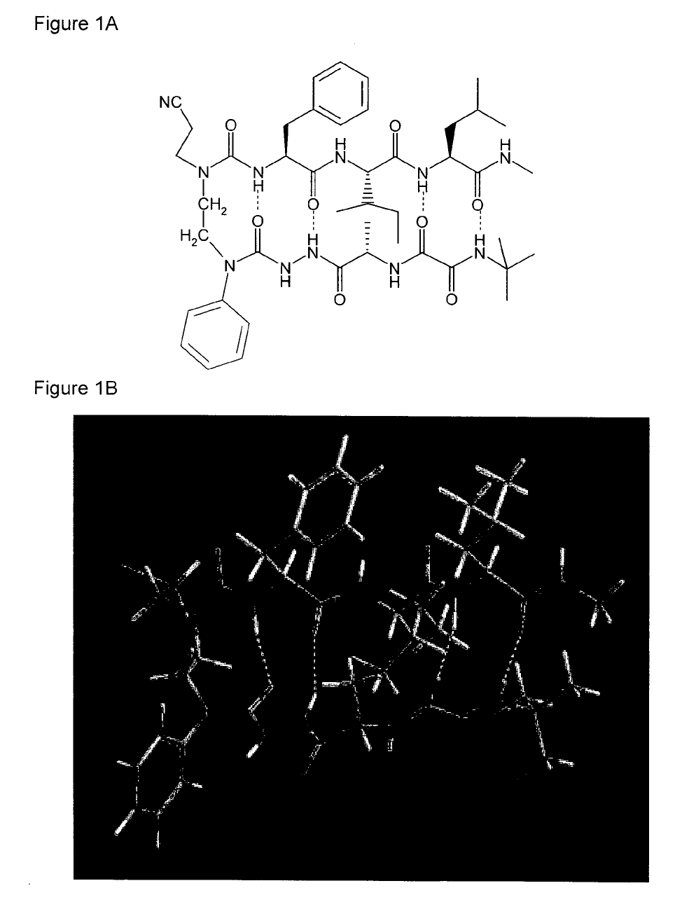

FIG. 2: Structures of a hydrogen bonded beta-sheet dimer compound, A: 2D, B: 3D.

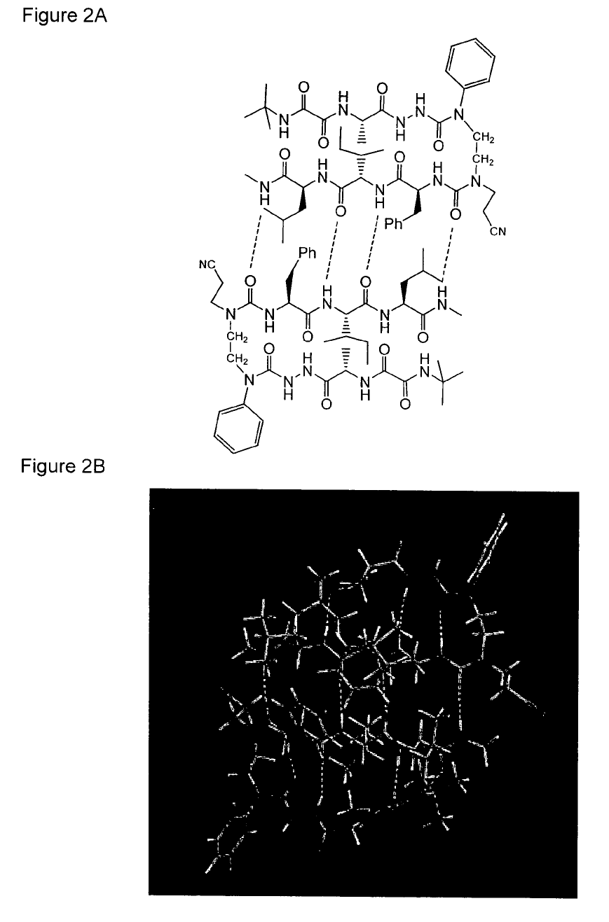

FIG. 3: Structures of beta-turns of a prolin amino acid analogon, A: 2D, B: 3D.

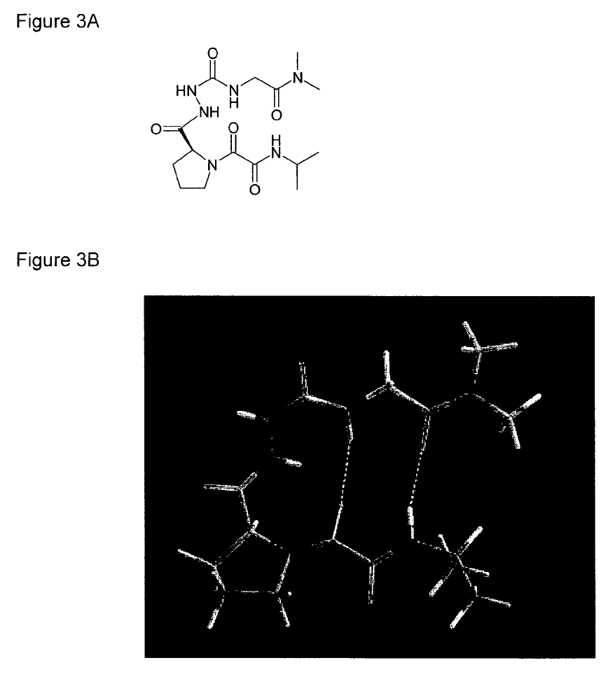

### EXAMPLES

### Abbreviations

Boc: t-Butyloxycarbonyl; DCC: Dicyclohexylcarbodiimide; DCM: DCM; DCHU: Dicyclohexylurea; DIPA: Diisopropylethylamine (=Hünig's base); DIC: Diisopropylcarbodiimide; EtOH: ethanol; EtOAc: ethyl acetate; EDCI.HCl Ethyldiisopropylcarbodiimide hydrochloride; Fmoc: Fluorenylmethyloxycarbonyl; HOBt: 1-Hydroxybenzotriazole; MeOH: methanol; THF: Tetrahydrofurane; TFA: trifluoroacetic acid; Z: Benzyloxycarbonyl.

### (S)-2-(methoxyoxalyl-amino)-3-phenyl-propionic acid

### Example 1

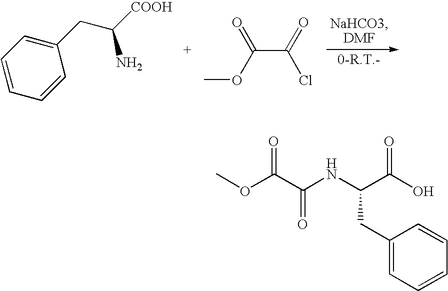

Mono-methyl oxalylchloride (3.34 mL, 36.32 mmol) was added drop-wise at 0° C. to the solution of L-phenylalanine (3.0 g, 18.16 mmol) and NaHCO₃ (7.63 g, 90.81 mmol) in DMF (15 mL). The reaction mixture was stirred further at rt for 2 h. DMF was removed by evaporation, the residue obtained was dissolved in water (50 mL), washed with diethyl ether (2×10 mL), acidified (pH 4) with 1 N HCL and extracted with EtOAc (3×15 mL). The combined organic layer was washed with brine (2×5 mL), dried (Na₂SO₄) and concentrated to give 2.58 g (56%) of a yellowish viscous oil. ¹H NMR (200 MHz, DMSO-d₆) δ (ppm) 9.06 (d, J=9.2 Hz, 1H), 7.15-7.31 (m, 5H), 4.43-4.54 (m, 1H), 3.76 (s, 3H), 2.97-3.21 (m, 2H).

### N—[(S)-1-Benzyl-2-(N′-t-butoxycarbonyl-hydrazino)-2-oxo-ethyl]-oxalamic acid methyl ester

### Example 2

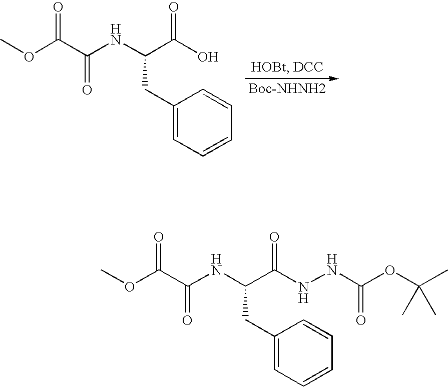

To the solution of (S)-2-(methoxy-oxalyl-amino)-3-phenyl-propionic acid (Example 1) (1.6 g, 6.37 mmol) in DCM (30 mL) was added HOBt (903.6 mg, 6.69 mmol) followed by DCC (1.38 g, 6.69 mmol) and stirred for 30 min at rt. To this Boc-carbazate (883.8 mg, 6.69 mmol) was added and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (10 mL), 10% NaHCO₃ (10 mL), brine (10 mL), dried (Na₂-SO₄) and concentrated to give 2.1 g of crude compound which was purified by column chromatography to give 1.64 g (70%) of a white solid; mp. 72-73° C.; [α]_{D}−28.6 (c 0.5, MeOH). 1H NMR (200 MHz, CDCl₃) δ (ppm) 8.2 (br s, 1H), 7.8 (br d, J=9.98 Hz, 1H), 7.19-7.35 (m, 5H), 6.56 (br s, 1H), 4.74-4.86 (m, 1H), 3.85 (s, 3H), 3.06-3.32 (m, 2H), 1.45 (s, 9H). 13C NMR (200 MHz, CDCl₃) δ (ppm) 169.53, 160.21, 156.38, 155.23, 135.75, 129.24, 128.69, 127.16, 82.04, 53.66, 53.2, 37.55, 28.06. IR (KBr) ν 4000-3292 (br.), 3031, 2980, 1742, 1688 (br.) cm⁻¹.

### N—[(S)-1-Benzyl-2-(N′-t-butoxycarbonyl-hydrazino)-2-oxo-ethyl]-oxalamic acid

### Example 3

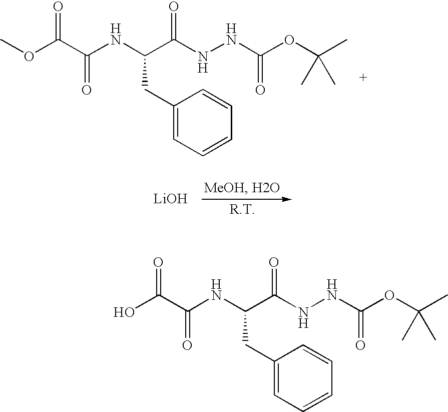

To the solution of N—[(S)-1-benzyl-2-(N′-t-butoxycarbonyl-hydrazino)-2-oxo-ethyl]-oxalamic acid methyl ester (Example 2) (520 mg, 1.42 mmol) in MeOH (10 mL) was added lithium hydroxide (38 mg, 1.57 mmol) followed by 2 drops of water. The reaction mixture was stirred at rt for 1 h. MeOH was evaporated and the residue obtained was dissolved in water (30 mL), washed with ether (2×10 mL). The aqueous layer was cooled in ice bath, acidified (pH=4) with 1N HCl and extracted with EtOAc (3×10 mL). The combined organic layer was washed with brine (10 mL), dried (Na₂SO₄) and concentrated to give 333 mg (66%) of an off-white solid; mp. 82-85° C. ¹H NMR (200 MHz, CDCl₃) δ (ppm) 9.32 (br, 1H), 8.9 (br s, 1H), 8.1 (br d, J=7.24 Hz, 1H), 7.14 (br, 5H), 6.96 (br, 1H), 4.75 (m, 1H), 2.95-3.23 (m, 2H), 1.36 (s, 9H). IR (KBr) ν 4000-3300 (br.), 3031, 2981, 2934, 1689 (br.) cm⁻¹.

### N-{(S)-1-Benzyl-2-[N′-(9H-fluoren-9-ylmethoxycarbonyl)-hydrazino]-2-oxo-ethyl}-oxalamic acid methyl ester

### Example 4

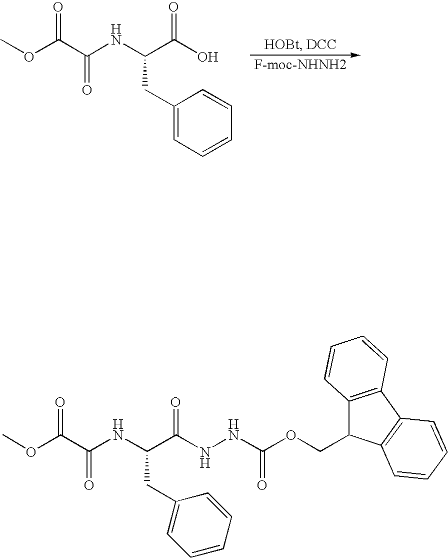

Following the procedure of Example 2 using (S)-2-(methoxyoxalyl-amino)-3-phenyl-propionic acid and Fmoc-carbazate the crude compound was obtained which was purified by column chromatography to give 975 mg (61%) of a white solid; mp. 91-93° C.; [α]_{D}−19.4 (c 0.5, MeOH). 1H NMR (200 MHz, DMSO-d₆) δ (ppm) 10.08 (br s, 1H), 9.38 (br s, 1H), 8.99 (d, J=8.4 Hz, 1H), 7.89 (d, J=7.04 Hz, 2H) 7.72 (d, J=6.26 Hz, 2H), 7.17-7.46 (m, 9H), 4.53-4.64 (m, 1H), 4.26-4.34 (m, 3H), 3.73 (s, 3H), 2.93-3.15 (m, 2H). 13C NMR (200 MHz, DMSO-d₆) δ (ppm) 169.98, 160.67, 156.82, 155.94, 143.61, 140.74, 137.44, 129.08, 128.19, 127.65, 126.99, 126.43, 125.11, 120.05, 66.24, 53.13, 52.70, 46.42, 36.62. IR (KBr) ν 4000-3275 (br.), 3030, 2954, 1734, 1704 (br.) cm⁻¹.

### (S)-2-t-Butoxycarbonylamino-3-phenyl-propionic acid

### Example 5

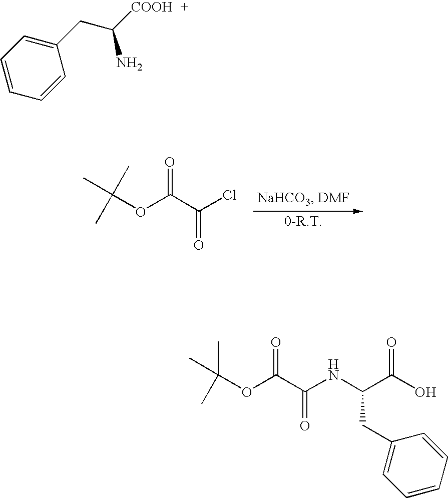

Following the procedure of Example 1 using mono-t-butyl oxalylchloride 250 mg of the crude compound as a colorless viscous oil was obtained that was used as such for further reactions.

### N′-((S)-2-t-Butoxycarbonylamino-3-phenyl-propionyl)-hydrazine carboxylic acid 9H-fluoren-9-ylmethyl ester

### Example 6

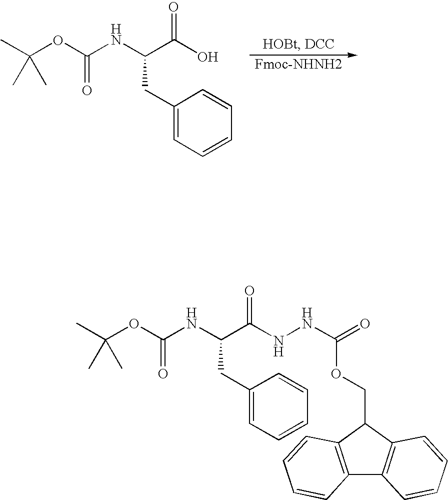

Following the procedure of Example 2 using Boc-L-Phenylalanine and Fmoc-carbazate 4.4 g of crude compound was obtained which was crystallized from CHCl₃-pet ether to give 3.6 g (95%) of a white solid; mp. 160-161° C. R_{f}=0.26 (1.5:3.5 EtOAc/pet ether); [α]_{D}−11.2 (c 0.5, MeOH). ¹H NMR (200 MHz, DMSO-d₆) δ (ppm) 9.94 (br s, 1H, d₂o exch.), 9.35 (br s, 1H, d₂o exch.), 7.88 (d, J=6.86 Hz, 2H), 7.73 (d, J=7.04 Hz, 2H), 7.17-7.45 (m, 9H), 6.93 (d, J=8.6 Hz, 1H, d₂o exch.), 4.28-4.31 (m, 4H), 2.69-3.0 (m, 2H), 1.27 (s, 9H). 13C NMR (200 MHz, DMSO-d₆) δ (ppm) 171.67, 155.95, 155.13, 143.58, 140.65, 137.94, 129.15, 127.95, 127.64, 127.06, 126.16, 125.24, 120.05, 79.09, 77.93, 66.11, 54.16, 46.44, 28.05. IR (KBr) ν 4000-3364, 3309, 3250, 3037, 3003, 2980, 1757, 1692, 1675, 1517 cm⁻¹.

### N-{(S)-1-Benzyl-2-[N′-(9H-fluoren-9-ylmethoxycarbonyl)-hydrazino]-2-oxo-ethyl}-oxalamic acid t-butyl ester

### Example 7

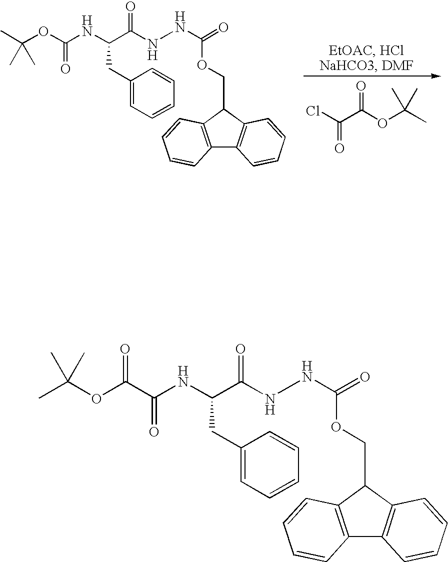

To the solution of N′-((S)-2-t-Butoxycarbonylamino-3-phenyl-propionyl)-hydrazine carboxylic acid 9H-fluoren-9-ylmethyl ester (Example 7) (1.2 g, 2.39 mmol) in EtOAc (10 mL), EtOAc saturated with HCl (10 mL) was added slowly with stirring at 0° C. The reaction mixture was stirred at rt for 30 min. Solvent was evaporated and the reaction mixture was dried under high vacuum. The residue obtained was dissolved in dry DMF (30 mL), NaHCO₃ (1.41 g, 16.75 mmol) was added to the reaction mixture, followed by t-butyl oxalylchloride (0.59 g, 3.59 mmol) at 0° C. under argon. The reaction mixture was stirred at rt for 30 min. The mixture was diluted with water (100 mL) and extracted with EtOAc (3×30 mL), the combined organic layer was washed with 1N HCl (30 mL) followed by 10% NaHCO₃ (30 mL) and water (3×30 mL) followed by brine (20 mL). The EtOAc layer was dried (Na₂SO₄) and concentrated to give 770 mg (60%) of a white powder; mp 106-108° C. R_{f}=0.29 (1.5:3.5 EtOAc/pet ether); [α]_{D}−18.0 (c 0.5, DMF). 1H NMR (200 MHz, CDCl₃) δ (ppm) 8.49 (br s, 1H, d₂o exch.), 7.64 (d, J=7.42 Hz, 3H, 1H, d₂o exch.), 7.46 (d, J=7.42 Hz, 2H), 7.14-7.31 (m, 9H), 6.96 (br s, 1H, d₂o exch.), 4.66-4.77 (m, 1H), 4.21-4.35 (m, 2H), 4.06-4.13 (m, 1H), 2.96-3.19 (m, 2H), 1.37 (s, 9H). 13C NMR (200 MHz, CDCl₃) δ (ppm) 170.02, 158.36, 157.78, 156.03, 143.46, 141.23, 135.69, 129.32, 128.74, 127.77, 127.22, 127.14, 125.14, 119.95, 85.01, 68.11, 53.35, 46.80, 37.59, 27.61. IR (KBr) ν 4000-3285, 3065, 2980, 1751, 1732, 1692, 1701, 1517, 1451, 1371, 1219, 1155, 1031, 840, 758, 740, cm⁻¹.

### N′-((S)-2-t-Butoxycarbonylamino-3-phenyl-propionyl)-hydrazine carboxylic acid benzyl ester

### Example 8

Following the procedure of Example 2 using Boc-L-Phenylalanine and z-carbazate 6.4 g of crude compound was obtained which was purified by column chromatography to give 4.2 g (89%) of an off-white solid; mp. 112-113° C. R_{f}=0.23 (1.5:3.5 EtOAc/pet ether); [α]_{D}−10.2 (c 0.5, MeOH). 1H NMR (200 MHz, CDCl₃) δ (ppm) 8.65 (br s, 1H, d₂O exch.), 7.12-7.22 (m, 11H), 5.27 (br s, 1H, d₂o exchangeable), 5.03 (s, 2H), 4.45 (m, 1H), 2.78-3.21 (m, 2H), 1.24 (s, 9H). 13C NMR (200 MHz, CDCl₃) δ (ppm) 171.65, 156.14, 155.79, 136.42, 135.64, 129.39, 128.50, 128.27, 128.14, 126.81, 80.42, 67.71, 53.99, 38.27, 28.2. IR (KBr) ν 4000-3372, 3313, 3002, 2971, 2929, 1762, 1688, 1672, 1628, 1526 cm⁻¹.

### N—[(S)-1-Benzyl-2-(N′-benzyloxycarbonyl-hydrazino)-2-oxo-ethyl]-oxalamic acid t-butyl ester (Z-NPheO-O-t-Bu

### Example 9

Following the procedure of Example 7 using N′-((S)-2-t-Butoxycarbonylamino-3-phenylpropionyl)-hydrazine carboxylic acid benzyl ester (Example 8) 770 mg (60%) of product obtained as a colorless viscous oil which after triturating with pentane gave white powder; mp. 72-7° C. Rf=0.29 (1.5:3.5 EtOAc/pet ether); [α]_{D}−23.0 (c 0.5, MeOH). ¹H NMR (200 MHz, CDCl₃) δ (ppm) 8.51 (br s, 1H, d₂o exch.), 7.71 (d, J=8.2 Hz, 1H, d₂o exch.), 7.24-7.31 (m, 10H), 6.95 (br s, 1H, d₂o exch.), 5.12 (s, 2H), 4.72-4.83 (m, 1H), 3.04-3.26 (m, 2H), 1.48 (s, 9H). 13C NMR (200 MHz, CDCl₃) δ (ppm) 169.97, 158.37, 157.70, 155.99, 135.70, 135.50, 129.33, 128.70, 128.52, 128.33, 128.14, 127.18, 84.94, 67.83, 53.29, 37.57, 27.60. IR (KBr) ν 4000-3293, 3063, 3032, 2982, 2934, 1732 (br.), 1703 (br.), 1519, 1498, 1455, 1371, 1219, 1154, 1028, 840, 742, 698 cm⁻¹.

### N—[(S)-1-Benzyl-2-(N′-benzyloxycarbonyl-hydrazino)-2-oxo-ethyl]-oxalamic acid methyl ester (Z-NPheO-OMe,

### Example 10

Following the procedure of Example 7 using N′-((S)-2-t-Butoxycarbonylamino-3-phenylpropionyl)-hydrazine carboxylic acid benzyl ester (Example 8) and methyl oxalylchloride 1.1 g (94%) of product was obtained as a colorless viscous oil which after triturating with pentane gave a white powder; mp. 87-89° C. Rf=0.42 (1:1 pet ether/EtOAc); [α]_{D}−24.6 (c 0.5, MeOH). 1H NMR (200 MHz, CDCl₃) δ (ppm) 8.52 (br s, 1H), 7.77 (br s, 1H), 7.14-7.22 (m, 11H), 5.02 (s, 2H), 4.73-4.76 (m, 1H), 3.67 (s, 3H), 2.85-3.36 (m, 2H). 13C NMR (200 MHz, CDCl₃) δ (ppm) 169.92, 160.07, 156.47, 156.07, 135.64, 135.47, 129.25, 128.68, 128.52, 128.36, 128.15, 127.16, 67.88, 53.63, 53.25, 37.59. IR (KBr) ν 4000-3285 (br.), 3031, 2953, 1742, 1683, 1525, 1498, 1456, 1218, 1028, 980, 741, 697 cm⁻¹.

### N′—[(S)-2-Benzyloxycarbonylamino-3-(1H-indol-3-yl)-propionyl]-hydrazine carboxylic acid 9H-fluoren-9-ylmethyl ester

### Example 11

Following the procedure of Example 2 using Z-L-tryptophan and Fmoc-carbazate 1.7 g of crude compound was obtained which was purified by column chromatography to give 1.39 g (81%) of a white solid; mp. 191-192° C.; [α]_{D}−30.2 (c 0.5, MeOH). ¹H NMR (200 MHz, DMSO-d₆) δ (ppm) 10.82 (s, 1H), 10.08 (br s, 1H), 9.37 (br s, 1H), 7.89 (d, J=7.04 Hz, 2H), 7.67-7.77 (m, 3H), 6.94-7.46 (m, 14H), 4.93 (s, 2H), 4.34 (m, 4H), 2.88-3.19 (m, 2H). 13C NMR (200 MHz, DMSO-d₆) δ (ppm) 171.79, 155.99, 155.76, 143.62, 140.68, 136.88, 136.04, 128.22, 127.67, 127.41, 127.09, 125.27, 123.99, 120.82, 120.08, 118.48, 118.2, 111.26, 109.87, 66.15, 65.21, 53.93, 46.46, 27.84. IR (KBr) ν 4000-3385, 3276, 3062, 2949, 1730, 1708, 1694, 1681, 1668, 1623 cm⁻¹.

### N′—[(S)-2-t-Butoxycarbonylamino-3-(1H-indol-3-yl)-propionyl]-hydrazine carboxylic acid 9H-fluoren-9-ylmethyl ester

### Example 12

Following the procedure of Example 2 using Boc-L-tryptophan and Fmoc-carbazate 1.56 g (87%) of product was obtained as a white solid, mp. 181-182° C. Rf=0.36 (1:1 pet ether/EtOAc); [α]_{D}−13.0 (c 0.5, MeOH). 1H NMR (200 MHz, DMSO-d₆) δ (ppm) 10.8 (s, 1H), 9.97 (br s, 1H), 9.36 (br s, 1H), 7.89 (d, J=7.04 Hz, 2H), 7.74 (d, J=8.0 Hz, 2H) 7.65 (d, J=7.24 Hz, 1H), 7.3-7.46 (m, 5H), 6.94-7.19 (m, 3H), 6.76 (d, J=8.8 Hz, 1H), 4.28 (m, 4H), 2.85-3.16 (m, 2H), 1.29 (s, 9H). 13C NMR (200 MHz, DMSO-d₆) δ (ppm) 171.96, 155.98, 155.10, 143.61, 140.69, 135.98, 127.67, 127.29, 127.08, 125.28, 123.80, 120.75, 120.11, 118.51, 118.11, 111.25, 109.93, 77.90, 59.72, 53.41, 46.48, 28.08, 27.79. IR (KBr) ν 4000-3328, 3052, 2977, 2933, 1748, 1674, 1626 cm⁻¹.

### N—[(S)-2-[N′-(9H-Fluoren-9-ylmethoxycarbonyl)-hydrazino]-1-(1H-indol-3-ylmethyl)-2-oxo-ethyl]-oxalamic acid t-butyl ester (Fmoc-NTrpO-O-t-Bu

### Example 13

Following the procedure of Example 7 using N′—[(S)-2-t-Butoxycarbonylamino-3-(1H-indol-3-yl)-propionyl]-hydrazine carboxylic acid 9H-fluoren-9-ylmethyl ester (Example 12),882 mg (83%) of product was obtained as a colorless viscous oil which after triturating with pentane gave off-white powder; mp. 157-159° C. Rf=0.24 (1:1 pet ether/EtOAc); [α]_{D}−37.2 (c 0.5, MeOH). 1H NMR (200 MHz, CDCl₃) δ (ppm) 8.4 (br s, 1H), 8.19 (br s, 1H), 7.65-7.81 (m, 4H), 7.52 (d, J=7.24 Hz, 2H), 6.96-7.4 (m, 9H), 4.72-4.82 (m, 1H), 4.12-4.41 (m, 3H), 3.28 (m, 2H), 1.46 (s, 9H). 13C NMR (200 MHz, CDCl₃) δ (ppm) 170.4, 158.43, 157.58, 156.06, 143.46, 143.39, 141.21, 136.12, 127.79, 127.16, 125.13, 123.95, 122.18, 119.97, 119.76, 118.53, 111.43, 109.23, 85.04, 68.01, 52.9, 46.76, 27.96, 27.59. IR (KBr) ν 4000-3336 (br.), 2979, 1695, 1507, 1451, 1371, 1219, 1153, 758, 740 cm⁻¹.

### (S)-3-(1H-Indol-3-yl)-2-(methoxyoxalyl-amino)-propionic acid

### Example 14

Following the procedure of Example 1 using L-tryptophan 185 mg (65%) of desired compound was obtained as a viscous oil. 1H NMR (200 MHz, DMSO-d₆) δ (ppm) 10.83 (s, 1H), 8.93 (d, J=8.6 Hz, 1H), 7.52 (d, J=8.02 Hz, 1H) 7.33 (d, J=7.24 Hz, 1H), 6.93-7.14 (m, 3H), 4.47-4.58 (m, 1H), 3.74 (s, 3H), 3.13-3.33 (m, 2H).

### N—[(S)-2-[N′-(9H-Fluoren-9-ylmethoxycarbonyl)-hydrazino]-1-(1H-indol-3-ylmethyl)-2-oxo-ethyl]-oxalamic acid methyl ester (Fmoc-NTrpO-OMe

### Example 15

Following the procedure of Example 2 using (S)-3-(1H-Indol-3-yl)-2-(methoxyoxalylamino)-propionic acid (Example 14) and F-moc-hydrazine 265 mg of crude compound was obtained which was purified by column chromatography to give 135 mg (46%) of a white solid, mp. 141-142° C.; [α]_{D}−37.2 (c 0.5, MeOH). 1H NMR (200 MHz, DMSO-d₆) δ (ppm) 10.86 (s, 1H), 10.18 (brs, 1H), 9.43 (br s, 1H), 8.88 (d, J=7.62 Hz, 1H), 7.95 (d, J=7.64 Hz, 2H), 7.78 (d, J=7.04 Hz, 2H), 7.68 (d, J=7.62 Hz, 1H), 7.35-7.51 (m, 5H), 6.98-7.23 (m, 3H), 4.67-4.74 (m, 1H), 4.32-4.43 (m, 3H), 3.77 (s, 3H), 3.13-3.32 (m, 2H). 13C NMR (200 MHz, DMSO-d₆) δ (ppm) 170.30, 160.75, 156.85, 155.98, 143.61, 140.69, 136.03, 127.67, 127.13, 125.29, 123.88, 120.94, 120.08, 118.35, 111.29, 109.34, 66.25, 52.80, 52.37, 46.40, 27.03. IR (KBr) ν 4000-3389 (br.), 3040 (br.), 1741, 1680 cm⁻¹.

### N′—[(S)-2-t-Butoxycarbonylamino-3-(1H-indol-3-yl)-propionyl]-hydrazine carboxylic acid benzyl ester

### Example 16

Following the procedure of Example 2 using Boc-L-tryptophan and Z-hydrazine 2.16 g (96%) of product was obtained as a viscous oil which after triturating with pentane gave off-white powder; mp. 88-89° C. Rf=0.42 (1:1 pet ether/EtOAc); [α]_{D}−18.2 (c 0.5, MeOH). 1H NMR (200 MHz, CDCl₃) δ (ppm) 8.35 (br s, 2H), 7.49 (d, J=7.44 Hz, 1H), 6.91-7.18 (m, 10H), 5.25 (br d, 1H), 4.94 (s, 2H), 4.45 (m, 1H), 3.1 (m, 2H), 1.26 (s, 9H). 13C NMR (200 MHz, CDCl₃) δ (ppm) 171.94, 156.21, 155.71, 135.97, 135.56, 128.52, 128.31, 128.1, 127.54, 123.67, 121.96, 119.47, 118.51, 111.29, 109.53, 80.51, 67.72. 53.79, 34.11, 28.2. IR (KBr) ν 4000-3303 (br.), 3036, 2977, 1687 (br.), 1499, 1457, 1393, 1367, 1226, 1165, 1049, 741, 697, cm⁻¹.

### N—[(S)-2-(N′-Benzyloxycarbonyl-hydrazino)-1-(1H-indol-3-ylmethyl)-2-oxo-ethyl]-oxalamic acid t-butyl ester (Z-NTrpO-O-t-Bu

### Example 17

Following the procedure of Example 7 using N′—[(S)-2-t-Butoxycarbonylamino-3-(1H-indol-3-yl)-propionyl]-hydrazine carboxylic acid benzyl ester (Example 16) 0.85 g (80%) of product was obtained as a colorless viscous oil which after triturating with pentane gave off-white powder. Rf=0.32 (2:1 pet ether/EtOAc); [α]_{D}−56.8 (c 0.5, MeOH). 1H NMR (200 MHz, CDCl₃) δ (ppm) 8.55 (br s, 1H), 8.26 (br s, 1H), 7.77 (d, J=8.02 Hz, 1H), 7.63 (d, J=7.42 Hz, 1H), 7.03-7.31 (m, 10H), 5.05 (s, 2H), 4.68-4.78 (m, 1H), 3.25 (m, 2H), 1.45 (s, 9H). 13C NMR (200 MHz, CDCl₃) δ (ppm) 170.47, 158.42, 157.53, 156.12, 136.12, 135.48, 128.54, 128.35, 128.10, 127.34, 124.05, 122.04, 119.66, 118.45, 111.47, 109.06, 85, 67.80, 52.92, 27.96, 27.58. IR (KBr) ν 4000-3328 (br.), 3058, 2981, 2934, 1736 (br.), 1692 (br.), 1517, 1457, 1371, 1219, 1153, 838, 742 cm⁻¹.

### N—[(S)-2-(N′-Benzyloxycarbonyl-hydrazino)-1-(1H-indol-3-ylmethyl)-2-oxo-ethyl]-oxalamic acid methyl ester (Z-NTrpO-OMe

### Example 18

Following the procedure of Example 7 using N′—[(S)-2-t-Butoxycarbonylamino-3-(1H-indol-3-yl)-propionyl]-hydrazine carboxylic acid benzyl ester (Example 16) and methyl-oxalylchloride 569 mg (58%) of product was obtained as a white solid; mp. 97-99° C. Rf=0.12 (1:1 EtOAc/Pentane); [α]_{D}−37.4 (c 0.5, MeOH). 1H NMR (200 MHz, DMSO-d₆) δ (ppm) 10.81 (s, 1H), 10.13 (s, 1H), 9.31 (s, 1H), 8.82 (d, J=7.24 Hz, 1H), 6.94-7.63 (m, 10H), 5.1 (s, 2H), 4.63 (m, 1H), 3.72 (s, 3H), 3.19 (m, 2H). 13C NMR (200 MHz, DMSO-d₆) δ (ppm) 170.25, 160.75, 156.77, 156, 136.53, 135.99, 128.33, 127.93, 127.7, 127.06, 123.79, 120.88, 118.28, 111.27, 109.38, 65.95, 52.77, 52.36, 27.13. IR (KBr) v 4000-3299 (br), 3034, 2954, 1739, 1702, 1679, 1524, 1457, 1436, 1342, 1266, 1221, 1027, 1010, 743, 697 cm⁻¹.

### Carbonic acid 2-bromo-benzyl ester 4-{(S)-2-t-butoxycarbonylamino-3-[N′-(9H-fluoren-9-ylmethoxycarbonyl)-hydrazino]-3-oxo-propyl}-phenyl ester

### Example 19

Following the procedure of Example 2 using Boc-L-Tyr(2-Br-Z)-OH and Fmoc-hydrazine 2.5 g of crude compound was obtained which was purified by column chromatography to give 2.1 g (94%) of a white solid; mp. 77-79° C. Rf=0.57 (1:1 pet ether/EtOAc); [α]_{D}−5.8 (c 0.5, MeOH). 1H NMR (200 MHz, DMSO-d₆) δ (ppm) 9.95 (br s, 1H), 9.37 (br s, 1H), 7.89 (d, J=7.04 Hz, 2H), 7.68-7.75 (m, 3H), 7.55-7.67 (m, 1H), 7.33-7.54 (m, 8H), 7.16 (d, J=8.4 Hz, 2H), 6.98 (d, J=8.02 Hz, 1H), 5.31 (s, 2H), 4.22-4.36 (m, 4H), 2.7-3.04 (m, 2H), 1.29 (s, 9H). 13C NMR (200 MHz, DMSO-d₆) δ (ppm) 171.56, 155.96, 155.17, 152.80, 149.25, 143.59, 140.69, 134.05, 132.66, 130.69, 130.29, 128.05, 127.66, 127.13, 125.29, 122.99, 120.68, 120.09, 78.04, 69.22, 63.25, 54.11, 46.44, 33.30, 28.10. IR (KBr) ν 4000-3309, 3250, 2976, 1763, 1717, 1684, 1627 cm⁻¹.

### N—{(S)-1-[4-(2-Bromo-benzyloxycarbonyloxy)-benzyl]-2-[N′-(9H-fluoren-9-ylmethoxycarbonyl)-hydrazino]-2-oxo-ethyl}-oxalamic acid methyl ester (FmocNTyr(2-Br-Z)O-OMe

### Example 20

Following the procedure of Example 7 using carbonic acid 2-bromo-benzyl ester 4-{(S)-2-t-butoxycarbonylamino-3-[N′-(9H-fluoren-9-ylmethoxycarbonyl)-hydrazino]-3-oxopropyl}-phenyl ester (Example 19) and methyl oxalylchloride 970 mg of crude compound was obtained which was purified by column chromatography (silica gel) to give 957 mg (88%) of a white powder; mp 205-206° C.; [α]_{D}−10.2 (c 0.5, DMF). 1H NMR (200 MHz, DMSO-d₆) δ (ppm) 10.10 (br s, 1H), 9.38 (br s, 1H), 9.04 (br d, J=9.2 Hz, 1H), 7.88 (d, J=7.62 Hz, 2H), 7.67-7.70 (m, 3H), 7.32-7.57 (m, 9H), 7.14 (d, J=9.18 Hz, 2H), 5.30 (s, 3H), 4.53-4.66 (m, 1H), 4.16-4.34 (m, 3H), 3.73 (s, 3H), 2.96-3.15 (m, 2H). 13C NMR (200 MHz, DMSO-d₆) δ (ppm) 169.82, 160.59, 156.87, 155.94, 152.76, 149.35, 143.61, 140.67, 135.47, 134.08, 132.76, 130.74, 130.20, 128.02, 127.64, 127.02, 125.24, 122.99, 120.82, 120.12, 69.21, 66.19, 53.01, 52.77, 46.49, 35.83. IR (KBr) ν 4000-3382, 3296, 3019, 2955, 1765, 1735, 1709, 1683, 1509 cm⁻¹.

### N-{(S)-1-[4-(2-Bromo-benzyloxycarbonyloxy)-benzyl]-2-[N′-(9H-fluoren-9-ylmethoxycarbonyl)-hydrazino]-2-oxo-ethyl}-oxalamic acid t-butyl ester (FmocNTyr(2-Br-Z)O—O-t-Bu

### Example 21

Following the procedure of Example 7 using carbonic acid 2-bromo-benzyl ester 4-{(S)-2-t-butoxycarbonylamino-3-[N′-(9H-fluoren-9-ylmethoxycarbonyl)-hydrazino]-3-oxopropyl}-phenyl ester (Example 19) and t-butyl oxalylchloride 440 mg (84%) of product was obtained as a colorless viscous oil which after triturating with pentane gave white powder; mp. 124-126° C. Rf=0.38 (1:1 pet ether/EtOAc); [α]_{D}−12.4 (c 0.5, MeOH). 1H NMR (200 MHz, CDCl₃) δ (ppm) 8.53 (br s, 1H), 7.71-7.75 (m, 3H), 7.47-7.62 (m, 4H), 7.18-7.41 (m, 8H), 7.04-7.13 (m, 3H), 5.35 (s, 2H), 4.73-4.83 (m, 1H), 4.16-4.47 (m, 3H), 3.05-3.27 (m, 2H), 1.48 (s, 9H). 13C NMR (200 MHz, CDCl₃) δ (ppm) 169.82, 158.38, 157.78, 156.01, 153.35, 150.21, 143.44, 141.23, 134.19, 133.66, 132.93, 130.48, 130.17, 130.08, 127.78, 127.63, 127.14, 125.12, 123.45, 121.22, 119.96, 85.14, 69.63, 68.1, 53.17, 46.81, 36.92, 27.61. IR (KBr) ν 4000-3291 (br.), 2979, 1761, 1695, 1509, 1451, 1372, 1220, 1153, 758, 740 cm⁻¹.

### Carbonic acid 4-[(S)-3-(N′-benzyloxycarbonyl-hydrazino)-2-t-butoxycarbonylamino-3-oxo-propyl]-phenyl ester 2-bromo-benzyl ester

### Example 22

Following the procedure of Example 2 using Boc-L-Tyr(2-Br-Z)-OH and Z-hydrazine 1.26 g (96%) of the product was obtained as a viscous oil which, after triturating with pentane, gave an off-white powder; mp. 64-66° C. Rf=0.78 (1:1 pet ether/EtOAc); [α]_{D}−15.6 (c 0.5, MeOH). 1H NMR (200 MHz, CDCl₃) δ (ppm) 8.61 (br s, 1H), 7.61 (d, J=7.82 Hz, 1H), 7.5 (dd, J=1.36, 1.18 Hz, 1H), 7.07-7.38 (m, 12H), 5.36 (br s, 3H), 5.13 (s, 2H), 4.51 (m, 1H), 2.89-3.19 (m, 2H), 1.36 (s, 9H). 13C NMR (200 MHz, CDCl₃) δ (ppm) 171.39, 156.1, 155.74, 153.41, 150.02, 135.57, 134.29, 134.23, 132.93, 130.5, 130.16, 130.08, 128.52, 128.32, 128.15, 127.63, 123.46, 121, 80.62, 69.59, 67.78, 53.9, 37.48, 28.20. IR (KBr) ν 4000-3287 (br), 3034, 2978, 2932, 1764, 1683 (br), 1509, 1379, 1368, 1219, 1163, 1028, 1018, 751 cm⁻¹.

### N-{(S)-2-(N′-Benzyloxycarbonyl-hydrazino)-1-[4-(2-bromo-benzyloxycarbonyloxy)benzyl]-2-oxo-ethyl}-oxalamic acid t-butyl ester (Z-NTyr(2-Br-Z)O—O-t-Bu

### Example 23

Following the procedure of Example 7 using carbonic acid 4-[(S)-3-(N′-benzyloxycarbonyl-hydrazino)-2-t-butoxycarbonylamino-3-oxo-propyl]-phenyl ester 2-bromo-benzyl ester (Example 22) and t-butyl oxalylchloride 0.49 g (94%) of product as obtained as a colorless viscous oil which after triturating with pentane gave an off-white powder; mp. 93-94° C. Rf=0.73 (1:1 Pentane/EtOAc); [α]_{D}−10.4 (c 0.5, MeOH). 1H NMR (200 MHz, CDCl₃) δ (ppm) 8.53 (br s, 1H), 7.74 (d, J=8.2 Hz, 1H), 7.61 (dd, J=1.16, 1.16 Hz, 1H), 7.5 (dd, J=1.56, 1.74 Hz, 1H), 7.0-7.39 (m, 12H), 5.35 (s, 2H), 5.12 (s, 2H), 4.7-4.81 (m, 1H), 2.95-3.25 (m, 2H), 1.48 (s, 9H). 13C NMR (200 MHz, CDCl₃) δ (ppm) 169.85, 158.4, 157.73, 156.02, 153.34, 150.18, 135.49, 134.20, 133.68, 132.93, 130.47, 130.17, 130.1, 128.53, 128.35, 128.15, 127.64, 123.46, 121.16, 85.06, 69.61, 67.85, 53.12, 36.91, 27.61. IR (KBr) ν 4000-3287 (br.), 3035, 2981, 1760 (br.), 1508, 1372, 1218, 838, 750, 696 cm⁻¹.

### N-{(S)-2-(N′-Benzyloxycarbonyl-hydrazino)-1-[4-(2-bromo-benzyloxycarbonyloxy)benzyl]-2-oxo-ethyl}-oxalamic acid methyl ester (Z-NTyr(2-Br-Z)O-OMe

### Example 24

Following the procedure of Example 7 using Carbonic acid 4-[(S)-3-(N′-benzyloxycarbonyl-hydrazino)-2-t-butoxycarbonylamino-3-oxo-propyl]-phenyl ester 2-bromo-benzyl ester (Example 22) and methyl-oxalylchloride 460 mg (94%) of product was obtained as a white solid; mp. 78-79° C. Rf=0.2 (1:1 EtOAc/Pentane); [α]_{D}−9.0 (c 0.5, MeOH). ¹H NMR (200 MHz, CDCl₃) δ (ppm) 8.69 (br s, 1H), 7.92 (d, J=7.82 Hz, 1H), 7.6 (dd, J=0.98, 0.98 Hz, 1H), 7.49 (dd, J=1.56, 1.58 Hz, 1H), 7.06-7.38 (m, 12H), 5.34 (s, 2H), 5.10 (s, 2H), 4.75-4.82 (m, 1H), 3.76 (s, 3H), 3.0-3.26 (m, 2H). 13C NMR (200 MHz, CDCl₃) δ (ppm) 169.81, 160.05, 156.54, 156.12, 153.4, 150.15, 135.47, 134.17, 133.65, 132.93, 130.42, 130.19, 130.1, 128.53, 128.37, 128.13, 127.64, 123.46, 121.14, 69.64, 67.89, 53.7, 53.09, 36.93. IR (KBr) ν 4000-3281 (br.), 3034, 2955, 1761, 1702, 1688, 1507, 1379, 1218, 1027, 1017, 750, 696 cm⁻¹.

### (S)-2-(Methoxyoxalyl-amino)-4-methyl-pentanoic acid

### Example 25

Following the procedure of Example 1 using L-Leucine 3.2 g (96%) of the desired product was obtained as a colorless viscous oil.

### N—[(S)-1-(N′-t-butoxycarbonyl-hydrazinocarbonyl)-3-methyl-butyl]-oxalamic acid methyl ester (Boc-NLeuO-OMe)

### Example 26

Following the procedure of Example 2 using (S)-2-(Methoxyoxalyl-amino)-4-methyl-pentanoic acid (Example 25) 4.5 g of crude compound was obtained which was purified by column chromatography to give 3.7 g (75%) of product as a white solid; mp. soften at 71° C. and melt at 83° C.; [α]_{D}−43.4 (c 0.5, MeOH). 1H NMR (200 MHz, CDCl₃) δ (ppm) 8.90 (br s, 1H), 7.87 (br d, J=8.6 Hz, 1H), 6.89 (br s, 1H), 4.57-4.68 (m, 1H), 3.80 (s, 3H) 1.51-1.69 (m, 3H), 1.37 (s, 9H), 0.87 (dd, J=5.66, 5.48 Hz, 6H). 13C NMR (200 MHz, CDCl₃) δ (ppm) 170.67, 160.43, 156.52, 155.36, 81.77, 53.64, 50.44, 40.64, 28.05, 24.54, 22.87, 21.66. IR (KBr) ν 4000-3292 (br.), 2960, 2873, 1742, 1687 (br.) cm⁻¹.

### N′-((S)-2-t-Butoxycarbonylamino-4-methyl-pentanoyl)-hydrazine carboxylic acid 9H-fluoren-9-ylmethyl ester

### Example 27

Following the procedure of Example 2 using Boc-L-Leucine monohydrate and F-moc-hydrazine crude compound was obtained which was purified by column chromatography to give 7.2 g (95%) of product as a white solid; mp. soften at 82° C. and melting at 92° C.; [α]_{D}−37.0 (c 0.5, MeOH). 1H NMR (200 MHz, CDCl₃) δ (ppm) 9.04 (br s, 1H), 7.74 (d, J=7.44 Hz, 2H), 7.58 (d, J=7.22 Hz, 2H), 7.22-7.41 (m, 5H), 5.24-5.30 (m, 1H), 4.38 (d, J=6.84 Hz, 2H), 4.18-4.32 (m, 1H), 1.58-1.84 (m, 3H), 1.54 (s, 9H), 0.95 (dd, J=5.28, 5.48 Hz, 6H). 13C NMR (200 MHz, CDCl₃) δ (ppm) 172.86, 156.21, 156.14, 143.54, 141.2, 127.72, 127.12, 125.2, 119.92, 80.51, 67.99, 51.3, 46.85, 41.15, 28.3, 24.57, 22.91, 21.84. IR (KBr) ν 4000-3287 (br.), 2958, 2871, 1685 (br.) cm⁻¹.

### N-{(S)-1-[N′-(9H-Fluoren-9-ylmethoxycarbonyl)-hydrazinocarbonyl]-3-methylbutyl}-oxalamic acid methyl ester (Fmoc-NLeuO-OMe

### Example 28

Following the procedure of Example 7 using N′-((S)-2-t-Butoxycarbonylamino-4-methylpentanoyl)-hydrazine carboxylic acid 9H-fluoren-9-ylmethyl ester (Example 27) and methyl oxalylchloride 3.7 g of crude compound was obtained which was purified by column chromatography (silica gel) to give 3.4 g (90%) of product as a white powder; mp soften at 82° C. and melt at 87.3° C.; [α]_{D}−42.0 (c 0.5, MeOH). 1H NMR (200 MHz, CDCl₃) δ (ppm) 8.85 (s, 1H), 7.62-7.72 (m, 3H), 7.47 (d, J=7.22 Hz, 2H), 7.13-7.32 (m, 5H), 4.56-4.68 (m, 1H), 4.29 (d, J=7.24, 2H), 4.02-4.14 (m, 1H), 3.72 (s, 3H) 1.5-1.79 (m, 3H), 0.84 (dd, J=5.48 Hz, 6H). 13C NMR (200 MHz, CDCl₃) δ (ppm) 170.95, 160.45, 156.47, 156.22, 143.55, 141.24, 127.79, 127.15, 125.23, 119.98, 68.11, 53.76, 50.68, 46.85, 40.7, 24.69, 22.9, 21.75. IR (KBr) ν 4000-3283 (br.), 2956, 2925, 2854, 1739, 1684 (br.) cm⁻¹.

### N-{(S)-1-[N′-(9H-Fluoren-9-ylmethoxycarbonyl)-hydrazinocarbonyl]-3-methylbutyl}-oxalamic acid t-butyl ester (Fmoc-NLeuO-O-t-Bu

### Example 29

Following the procedure of Example 7 using N′-((S)-2-t-Butoxycarbonylamino-4-methylpentanoyl)-hydrazine carboxylic acid 9H-fluoren-9-ylmethyl ester (Example 27) and t-butyl oxalylchloride 1.0 g (88%) of product was obtained as a colorless viscous oil which after triturating with pentane gave an off-white powder; mp. 91-93° C. Rf=0.63 (1:1 pet ether/EtOAc); [α]_{D}−35.2 (c 0.5, MeOH). 1H NMR (200 MHz, CDCl₃) δ (ppm) 8.91 (br s, 1H), 7.55-7.76 (m, 5H), 7.23-7.41 (m, 4H), 7.12 (br s, 1H), 4.66-4.74 (m, 1H), 4.17-4.45 (m, 3H), 1.19-1.84 (m, 12H), 0.86-0.97 (m, 6H). 13C NMR (200 MHz, CDCl₃) 6 (ppm) 171.04, 158.71, 157.81, 156.1, 143.49, 141.21, 127.76, 127.13, 125.16, 119.94, 85.07, 68.18, 50.56, 46.8, 40.75, 27.62, 24.62, 22.76, 21.99. IR (KBr) ν 4000-3286 (br.), 2958, 1703, 1684, 1520, 1451, 1370, 1304, 1217, 1155, 759, 740 cm⁻¹.

### N′-((S)-2-t-Butoxycarbonylamino-4-methyl-pentanoyl)-hydrazine carboxylic acid benzyl ester

### Example 30

Following the procedure of Example 2 using Boc-L-Leu and Z-hydrazine 2.16 g (94%) of product was obtained as a viscous oil which after triturating with pentane gave off-white powder; mp. 57-59° C. Rf=0.76 (1:1 pet ether/EtOAc); [α]_{D}−42.6 (c 0.5, MeOH). 1H NMR (200 MHz, CDCl₃) δ (ppm) 8.96 (br s, 1H), 7.31 (s, 6H), 5.21 (br s, 1H), 5.12 (s, 2H), 4.3 (m, 1H), 1.51-1.71 (m, 3H), 1.4 (s, 9H), 0.88-0.93 (m, 6H). 13C NMR (200 MHz, CDCl₃) δ (ppm) 172.85, 156.23, 156.04, 135.63, 128.46, 128.24, 128.12, 80.49, 67.64, 51.21, 41.16, 28.24, 24.5, 22.85, 21.82. IR (KBr) ν 4000-3296 (br.), 2959, 1683 (br.), 1521, 1393, 1368, 1221, 1166, 1047, 741, 696, cm⁻¹.

### N—[(S)-1-(N′-Benzyloxycarbonyl-hydrazinocarbonyl)-3-methyl-butyl]-oxalamic acid t-butyl ester (Z-NLeuO-O-t-Bu

### Example 31

Following the procedure of Example 7 using N′-((S)-2-t-Butoxycarbonylamino-4-methylpentanoyl)-hydrazine carboxylic acid benzyl ester (Example 30) and t-butyl oxalylchloride 0.84 g (78%) of product was obtained as a colorless viscous oil which after triturating with pentane gave an off-white powder; mp. 69-71° C. Rf=0.72 (1:1 pentane/EtOAc); [α]_{D}−41.6 (c 0.5, MeOH). 1H NMR (200 MHz, CDCl₃) δ (ppm) 8.85 (br s, 1H), 7.66 (brs, 1H), 7.31 (s, 5H), 7.04 (brs, 1H), 5.18 (s, 2H), 4.59-4.63 (m, 1H), 1.61-1.82 (m, 3H), 1.51 (s, 9H), 0.90 (br s, 6H). 13C NMR (200 MHz, CDCl₃) δ (ppm) 171.02, 158.73, 157.72, 156.07, 135.52, 128.5, 128.29, 128.15, 84.95, 67.78, 50.51, 40.78, 27.61, 24.55, 22.71, 21.94. IR (KBr) ν 4000-3294 (br.), 2960, 1685 (br.), 1523, 1370, 1306, 1218, 1156, 842, 741, 697 cm⁻¹.

### N—[(S)-1-(N′-Benzyloxycarbonyl-hydrazinocarbonyl)-3-methyl-butyl]-oxalamic acid methyl ester (Z-NLeuO-OMe

### Example 32

Following the procedure of Example 7 using N/-((S)-2-t-Butoxycarbonylamino-4-methylpentanoyl)-hydrazine carboxylic acid benzyl ester (Example 30) and methyl-oxalylchloride 771 mg (88%) of product was obtained as a white solid. Rf=0.24 (1:1 EtOAc/Pentane); [α]_{D}−43.8 (c 0.5, MeOH). 1H NMR (200 MHz, CDCl₃) δ (ppm) 8.97 (br s, 1H), 7.82 (brs, 1H), 7.3 (brs, 6H), 5.1 (s, 2H), 4.63-4.66 (m, 1H), 3.79 (s, 3H), 1.68 (m, 3H), 0.89 (brs, 6H). 13C NMR (200 MHz, CDCl₃) δ (ppm) 171.99, 160.26, 156.62, 156.16, 135.5, 128.5, 128.31, 128.14, 67.8, 53.65, 50.55, 40.69, 24.55, 22.73, 21.74. IR (KBr) ν 4000-3287 (br), 3036, 2958, 1686 (br), 1525, 1456, 1217, 1043, 986, 741, 697 cm⁻¹.

### (S)-2-[N′-(9H-Fluoren-9-ylmethoxycarbonyl)-hydrazinocarbonyl]-pyrrolidine-1-carboxylic acid t-butyl ester

### Example 33

To the solution of Boc-L-Proline succinamide ester (0.5 g, 1.6 mmol) in DCM (15 mL) was added Fmoc-hydrazine (0.41 g, 1.6 mmol) was added and the mixture was stirred overnight at rt. The reaction mixture was diluted with EtOAc (50 mL) and washed with 1N HCl (15 mL),10% NaHCO₃ (15 mL), brine (15 mL), dried (Na₂SO₄) and concentrated

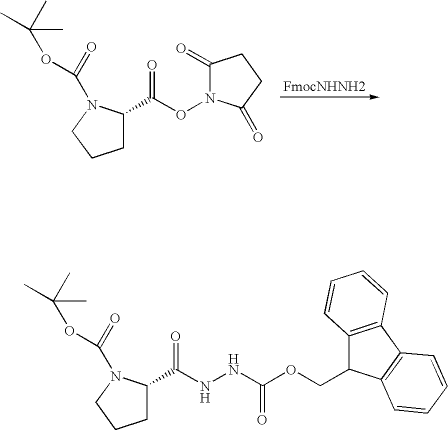

to give 701 mg (96%) of product as a white solid; mp. 81-82° C. Rf=0.3 (1:1 EtOAc/Pentane); [α]_{D}−59.8 (c 0.5, MeOH). 1H NMR (200 MHz, CDCl₃) δ (ppm) 8.85 (br s, 1H), 7.65 (d, J=7.24 Hz, 2H), 7.5 (d, J=7.24 Hz, 2H), 7.11-7.33 (m, 5H), 4.25-4.34 (m, 3H), 4.1-4.17 (m, 1H), 3.37 (m, 2H), 1.69-2.08 (m, 4H), 1.38 (s, 9H). 13C NMR (200 MHz, CDCl₃) δ (ppm) 172.02, 171.17, 156.07, 143.56, 141.23, 127.73, 127.11, 125.15, 119.93, 80.85, 67.92, 58.38, 47.1, 46.9, 28.36, 24.42, 14.17. IR (KBr) ν 4000-3277 (br), 2977, 1696 (br), 1451, 1405, 1366, 1246, 1164, 759, 740 cm⁻¹.

### (S)-2-(N′-Benzyloxycarbonyl-hydrazinocarbonyl)-pyrrolidine-1-carboxylic acid t-butyl ester

### Example 34

Following the procedure of Example 33 using Z-hydrazine 1.1 g (94%) of product was obtained as a sticky mass. Rf=0.28 (1:1 EtOAc/Pentane); [α]_{D}−82.0 (c 0.5, MeOH). ¹H NMR (200 MHz, CDCl₃) δ (ppm) 8.76 (br s, 1H), 7.25 (s, 5H), 6.94 (br s, 1H), 5.07 (s, 2H), 4.22 (br s, 1H), 3.32 (br s, 2H), 1.68-2.21 (m, 4H), 1.37 (s, 9H). 13C NMR (200 MHz, CDCl₃) δ (ppm) 171.99, 156.02, 135.66, 128.48, 128.25, 128.1, 80.79, 67.64, 58.24, 47.03, 28.32, 24.41, 14.15. IR (KBr) ν 4000-3280 (br), 2979, 1699 (br), 1456, 1404, 1367, 1219, 1164, 742, 697 cm⁻¹.

### {(S)-2-[N′-(9H-Fluoren-9-ylmethoxycarbonyl)-hydrazinocarbonyl]-pyrrolidin-1-yl}-oxo-acetic acid t-butyl ester (Fmoc-NProO-O-t-Bu

### Example 35

Following the procedure of Example 7 using (S)-2-[N′-(9H-Fluoren-9-ylmethoxycarbonyl)-hydrazinocarbonyl]-pyrrolidine-1-carboxylic acid t-butyl ester (Example 33) and t-butyl oxalylchloride 637 mg (99%) of product was obtained as a white solid; mp. 86-87° C. Rf=0.13 (1:1 EtOAc/Pentane); [α]_{D}−68.6 (c 0.5, MeOH). 1H NMR (200 MHz, CDCl₃) δ (ppm) 8.83 (s, 1H), 7.73 (d, J=7.04 Hz, 2H), 7.58 (d, J=7.24 Hz, 2H), 7.17-7.41 (m, 5H), 4.57-4.74 (m, 1H), 4.37-4.41 (m, 2H), 4.19-4.26 (m, 1H), 3.63-3.81 (m, 2H), 1.94-2.4 (m, 4H), 1.53 (s, 9H). (small percentage of other rotamer also observed). 13C NMR (200 MHz, CDCl₃) δ (ppm) 170.47, 160.85, 160.56, 156.1, 143.59, 141.21, 127.71, 127.13, 125.21, 119.91, 84.81, 68.01, 58.49, 48.22, 46.85, 27.86, 27.74, 24.91. IR (KBr) ν 4000-3282 (br), 2980, 1733, 1700, 1656, 1652, 1451, 1370, 1252, 1149, 759, 740 cm⁻¹.

### [(S)-2-(N′-Benzyloxycarbonyl-hydrazinocarbonyl)-pyrrolidin-1-yl]-oxo-acetic acid t-butyl ester (Z-NProO-O-t-Bu

### Example 36

Following the procedure of Example 7 using (S)-2-(N′-Benzyloxycarbonyl-hydrazinocarbonyl)-pyrrolidine-1-carboxylic acid t-butyl ester (Example 34) and t-butyl oxalylchloride 807 mg (99%) of product was obtained as a white solid; mp. 51-52° C. Rf=0.15 (1:1 EtOAc/Pentane); [α]_{D}−70.8 (c 0.5, MeOH).1H NMR (200 MHz, CDCl₃) δ (ppm) 8.76 (br s, 1H), 7.31 (s, 5H), 7.12 (br s, 1H), 5.12 (s, 2H), 4.5-4.67 (m, 1H), 3.6-3.66 (m, 2H), 1.85-2.26 (m, 4H), 1.53 (s, 9H) (minor percentage of another rotamer also observed). 13C NMR (200 MHz, CDCl₃) δ (ppm) 170.47, 160.81, 160.39, 156.06, 135.73, 128.46, 128.18, 128.07, 84.69, 67.59, 58.46, 48.16, 27.85, 27.68, 24.81. IR (KBr) ν 4000-3290 (br), 2982, 1733 (br), 1651 (br), 1456, 1371, 1253, 115, 742, 698 cm⁻¹.

### [(S)-2-(N′-Benzyloxycarbonyl-hydrazinocarbonyl)-pyrrolidin-1-yl]-oxo-acetic acid methyl ester (Z-NProO-OMe

### Example 37

Following the procedure of Example 7 using (S)-2-(N′-Benzyloxycarbonyl-hydrazinocarbonyl)-pyrrolidine-1-carboxylic acid t-butyl ester (Example 34) and mono-methyl oxalylchloride 620 mg (86%) of product was obtained as a sticky mass.

### N′-((DL)-2-t-Butoxycarbonylamino-3-phenyl-propionyl)-hydrazine carboxylic acid 9H-fluoren-9-ylmethyl ester

### Example 38

Following the procedure of Example 2 using Boc-DL-phenylalanine and F-moc-hydrazine 514 mg (67%) of product was obtained as a white solid.

### N′-((DL)-2-t-Butoxycarbonylamino-3-phenyl-propionyl)-hydrazine carboxylic acid benzyl ester

### Example 39

Following the procedure of Example 2 using Boc-DL-phenylalanine and Z-hydrazine 617 mg (98%) of product was obtained as a white solid. N-{(DL)-1-Benzyl-2-[N′-(9H-fluoren-9-ylmethoxycarbonyl)-hydrazino]-2-oxo-ethyl}-oxalamic acid t-butyl ester (Fmoc-N(DL)PheO-O—O-tBu; Example 40): Following the procedure of Example 7 using N′-((DL)-2-t-Butoxycarbonylamino-3-phenyl-propionyl)-hydrazine carboxylic acid 9H-fluoren-9-ylmethyl ester (Example 38) and t-butyl oxalylchloride 450 mg of crude compound was obtained which was purified by column chromatography to give 278 mg (52%) of a white solid.

### N—[(DL)-1-Benzyl-2-(N′-benzyloxycarbonyl-hydrazino)-2-oxo-ethyl]-oxalamic acid t-butyl ester (Z-N(DL)PheO-O—O-tBu

### Example 41

Following the procedure of Example 7 using N′-((DL)-2-t-Butoxycarbonylamino-3-phenyl-propionyl)-hydrazine carboxylic acid benzyl ester (Example 39) and t-butyl oxalylchloride 430 mg of crude compound was obtained which was purified by column chromatography to give 304 mg (56%) of a white solid.

### N′-((R)-2-t-Butoxycarbonylamino-3-phenyl-propionyl)-hydrazine carboxylic acid 9H-fluoren-9-ylmethyl ester

### Example 42

Following the procedure of Example 2 using Boc-D-phenylalanine and F-moc-hydrazine 760 mg of the crude compound which was purified by column chromatography to give 710 mg (93%) of a white solid. Rf=0.26 (1.5:3.5 EtOAc/pet ether); [α]_{D}+10.8 (c 0.5, MeOH). 1H NMR (200 MHz, DMSO-d₆) δ (ppm) 9.94 (br s, 1H, d₂o exch.), 9.35 (br s, 1H, d₂o exch.), 7.88 (d, J=6.86 Hz, 2H), 7.73 (d, J=7.04 Hz, 2H), 7.17-7.45 (m, 9H), 6.93 (d, J=8.6 Hz, 1H, d₂o exch.), 4.28-4.31 (m, 4H), 2.69-3.0 (m, 2H), 1.27 (s, 9H). 13C NMR (200 MHz, DMSO-d₆) δ (ppm) 171.67, 155.95, 155.13, 143.58, 140.65, 137.94, 129.15, 127.95, 127.64, 127.06, 126.16, 125.24, 120.05, 79.09, 77.93, 66.11, 54.16, 46.44, 28.05.

### N-{(R)-1-Benzyl-2-[N′-(9H-fluoren-9-ylmethoxycarbonyl)-hydrazino]-2-oxo-ethyl}-oxalamic acid t-butyl ester (Fmoc-N(D)PheO-O—O-tBu

### Example 43

Following the procedure of Example 7 using N′-((R)-2-t-Butoxycarbonylamino-3-phenylpropionyl)-hydrazine carboxylic acid 9H-fluoren-9-ylmethyl ester (Example 42) and t-butyl oxalylchloride 835 mg (79%) of product was obtained as a white powder. Rf=0.29 (1.5:3.5 EtOAc/pet ether); [α]_{D}+18.4 (c 0.5, DMF). 1H NMR (200 MHz, CDCl₃) δ (ppm) 8.49 (br s, 1H, d₂o exch.), 7.64 (d, J=7.42 Hz, 3H, 1H, d₂o exch.), 7.46 (d, J=7.42 Hz, 2H), 7.14-7.31 (m, 9H), 6.96 (br s, 1H, d₂o exch.), 4.66-4.77 (m, 1H), 4.21-4.35 (m, 2H), 4.06-4.13 (m, 1H), 2.96-3.19 (m, 2H), 1.37 (s, 9H). 13C NMR (200 MHz, CDCl3) δ (ppm) 170.02, 158.36, 157.78, 156.03, 143.46, 141.23, 135.69, 129.32, 128.74, 127.77, 127.22, 127.14, 125.14, 119.95, 85.01, 68.11, 53.35, 46.80, 37.59, 27.61.

### (S)-6-Benzyl-[1,2,5]triazepane-3,4,7-trione

### Example 44

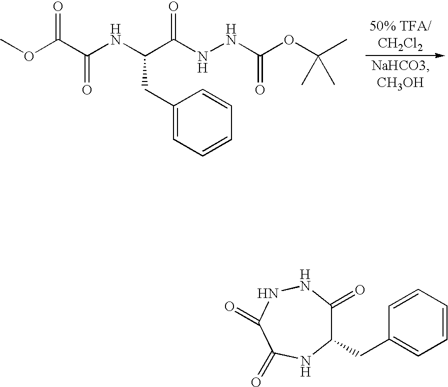

To the solution of N—[(S)-1-Benzyl-2-(N′-t-butoxycarbonyl-hydrazino)-2-oxo-ethyl]-oxalamic acid methyl ester (Example 2) (1.0 g, 2.74 mmol) in CH₂Cl₂ (15 mL) was added TFA (15 mL) drop-wise, the reaction mixture was further stirred at rt for 30 min under argon. The reaction mixture was then evaporated oxalylchloride and dried well under high vacuum. The residue obtained was dissolved in dry CH₂Cl₂ (15 mL) and washed with 10% NaHCO₃. The aqueous solution was extracted with CH₂Cl₂ (3×15 mL), the combined organic layer was dried over Na₂SO₄ and evaporated under reduced pressure. The residue obtained was refluxed overnight in MeOH (15 mL) under nitrogen. The solvent was evaporated and the crude compound obtained was purified by column chromatography to give 350 mg (54%) of desired compound as a white solid. 1H NMR (200 MHz, CDCl₃) δ (ppm) 8.3 (br s, 1H), 8.1 (br s, 1H), 7.1-7.4 (m, 5H), 6.7 (br s, 1H), 4.7-4.9 (m, 1H), 3.04-3.36 (m, 2H). 13C NMR (200 MHz, CDCl₃) δ (ppm) 171.83, 159.2, 158.6, 135.7, 129.23, 128.67, 127.1, 53.1, 37.5.

### N′-((S)-2-t-Butoxycarbonylamino-propionyl)-hydrazine carboxylic acid 9H-fluoren-9-ylmethyl ester

### Example 45

Following the procedure of Example 2 using Boc-L-alanine and Fmoc-hydrazine 4.4 g (97%) of product was obtained as a white solid; mp. 71-72° C. Rf=(1:1 Pentane/EtOAc); [α]_{D}−36.0 (c 0.5, MeOH).1H NMR (200 MHz, CDCl₃) δ (ppm) 8.78 (br s, 1H), 7.74 (d, J=7.42 Hz, 2H), 7.57 (d, J=7.44 Hz, 2H), 7.23-7.41 (m, 5H), 5.31 (br s, 1H), 4.39 (d, J=7.02 Hz, 2H), 4.18-4.33 (m, 1H), 1.38-1.44 (m, 12H). 13C NMR (200 MHz, CDCl₃) δ (ppm) 172.88, 156.24, 155.77, 143.48, 141.22, 127.74, 127.11, 125.13, 119.93, 80.56, 67.98, 48.45, 46.85, 28.29, 18.09. IR (KBr) ν 4000-3293 (br.), 2979, 2934, 1668 (br.), 1506, 1451, 1368, 1247, 1167, 1046, 759, 740 cm⁻¹.

### N—{(S)-2-[N′-(9H-Fluoren-9-ylmethoxycarbonyl)-hydrazino]-1-methyl-2-oxo-ethyl}-oxalamic acid t-butyl ester (Fmoc-NAlaO-O—O-tBu

### Example 46

Following the procedure of Example 7 using N′-((S)-2-t-Butoxycarbonylaminopropionyl)-hydrazine carboxylic acid 9H-fluoren-9-ylmethyl ester (Example 45) and t-butyl oxalylchloride 3.8 g (93%) of product was obtained as a white solid; mp. 79-80° C. Rf=(1:1 Pentane/EtOAc); [α]_{D}−31.4 (c 0.5, MeOH). 1H NMR (200 MHz, CDCl₃) δ (ppm) 8.74 (br s, 1H), 7.63-7.72 (m, 3H), 7.47 (d, J=7.24 Hz, 2H), 7.09-7.32 (m, 5H), 4.47-4.61 (m, 1H), 4.3 (d, J=7.04 Hz, 2H), 4.1-4.15 (m, 1H), 1.37-1.43 (m, 12H). 13C NMR (200 MHz, CDCl₃) δ (ppm) 171.31, 158.62, 157.62, 156.17, 143.45, 141.22, 127.77, 127.12, 125.11, 119.95, 85.06, 68.08, 47.84, 46.81, 27.63, 17.65. IR (KBr) ν 4000-3290 (br.), 2982, 2936, 1732, 1692 (br.), 1520, 1451, 1371, 1296, 1220, 1156, 759, 741 cm⁻¹.

### N-{(S)-2-[N′-(9H-Fluoren-9-ylmethoxycarbonyl)-hydrazino]-1-methyl-2-oxo-ethyl}-oxalamic acid (Fmoc-NAlaOOH

### Example 47

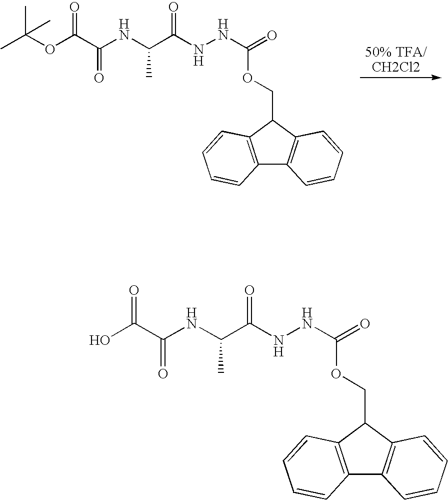

To the solution of N-{(S)-2-[N′-(9H-Fluoren-9-ylmethoxycarbonyl)-hydrazino]-1-methyl-2-oxo-ethyl}-oxalamic acid t-butyl ester (Example 46) (1.0 g, 2.21 mmol) in DCM (15 mL), TFA (15 mL) was added slowly with stirring at 0° C. The reaction mixture was stirred at rt for 30 min. Solvent was evaporated and well dried under high vacuum to give 870 mg of crude compound which after crystallization from EtOAc-pet ether gave 752 mg (85%) of desired compound as a white solid. 1H NMR (200 MHz, DMSO-d₆) δ (ppm) 9.92 (s, 1H), 9.33 (s, 1H), 8.73 (d, J=7.82 Hz, 1H), 7.87 (d, J=7.02 Hz, 2H), 7.71 (d, J=7.02 Hz, 2H), 7.28-7.44 (m, 4H), 4.27-4.4 (m, 4H), 1.33 (d, J=6.84 Hz, 3H). 13C NMR (200 MHz, DMSO-d₆) δ (ppm) 171.08, 161.67, 157.86, 155.88, 143.56, 140.66, 127.65, 127.06, 125.21 120.07, 66.12, 47.22, 46.43, 17.72.

### N—((S)-1-Hydrazinocarbonyl-ethyl)-oxalamic acid t-butyl ester (NAlaO-O-t-Bu

### Example 48

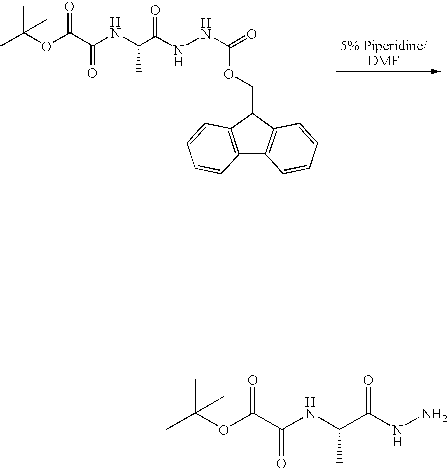

The solution of N-{(S)-2-[N′-(9H-Fluoren-9-ylmethoxycarbonyl)-hydrazino]-1-methyl-2-oxo-ethyl}-oxalamic acid t-butyl ester (Example 46) (2.1 g, 4.63 mmol) in 5% piperidine in DMF (15 mL) was stirred at rt for 20 min. Solvent was evaporated and the reaction mixture was well dried under high vacuum. The crude compound obtained was purified by column chromatography to give 137 mg (12%) of product thick sticky mass. 1H NMR (200 MHz, CDCl₃) δ (ppm) 7.81 (d, J=7.82 Hz, 1H), 4.37-4.51 (m, 1H), 4.06 (br s, 2H), 1.48 (s, 9H), 1.36 (d, J=7.04 Hz, 3H).

### N′-((S)-2-t-Butoxycarbonylamino-propionyl)-hydrazine carboxylic acid benzyl ester

### Example 49

Following the procedure of Example 2 using Boc-L-alanine and Z-hydrazine 6.9 g (96%) of product was obtained as a white solid; mp. 130-131° C. Rf=(pet ether/EtOAc); [α]_{D}−43.6 (c 0.5, MeOH). 1H NMR (200 MHz, CDCl₃-2 drops DMSO-d₆) δ (ppm) 9.37 (bs, 1H), 8.29 (bs, 1H), 7.3 (s, 5H), 5.72 (bs, 1H), 5.1 (s, 2H), 4.23 (bs, 1H), 1.4 (s, 9H), 1.31 (d, J=6.64 Hz, 3H). 13C NMR (200 MHz, CDCl₃-2 drops DMSO-d₆) δ (ppm) 177.7, 161.11, 160.02, 140.87, 133.15, 132.84, 132.75, 84.19, 71.77, 53.33, 33.08, 23.55.

### N—[(S)-2-(N′-Benzyloxycarbonyl-hydrazino)-1-methyl-2-oxo-ethyl]-oxalamic acid t-butyl ester (Z-NAlaO-O-t-Bu

### Example 50

Following the procedure of Example 7 using N′-((S)-2-t-Butoxycarbonylaminopropionyl)-hydrazine carboxylic acid benzyl ester (Example 49) and t-butyl oxalylchloride 2.8 g (86%) of product was obtained as a white solid; mp. 49-51° C. Rf=(1:1 Pentane/EtOAc); [α]_{D}−44.0 (c 0.5, MeOH). 1H NMR (200 MHz, CDCl₃) δ (ppm) 8.87 (br s, 1H), 7.84 (d, J=7.64 Hz, 1H), 7.3 (s, 5H), 7.21 (s, 1H), 5.1 (s, 2H), 4.51-4.65 (m, 1H), 1.5 (s, 9H), 1.42 (d, J=6.66 Hz, 3H). 13C NMR (200 MHz, CDCl₃) δ (ppm) 171.43, 158.65, 157.53, 156.2, 135.52, 128.5, 128.31, 128.12, 84.93, 67.78, 47.83, 27.61, 17.71. IR (KBr) ν 4000-3296 (br.), 2984, 2938, 1733, 1699 (br.), 1517, 1456, 1371, 1297, 1219, 1156, 840, 740, 697 cm⁻¹.

### N—[(S)-2-(N′-Benzyloxycarbonyl-hydrazino)-1-methyl-2-oxo-ethyl]-oxalamic acid methyl ester (Z-NAlaO-OMe

### Example 51

Following the procedure of Example 7 using N′-((S)-2-t-Butoxycarbonylaminopropionyl)-hydrazine carboxylic acid benzyl ester (Example 49) and mono-methyl oxalylchloride 640 mg (22%) of product was obtained as a white solid; mp. 51-52° C. Rf=0.37 (1:1 Pentane/EtOAc); [α]_{D}−47.6 (c 0.5, MeOH). 1H NMR (200 MHz, CDCl₃) δ (ppm) 8.93 (s, 1H), 8.0 (d, J=7.24 Hz, 1H), 7.35 (s, 1H), 7.3 (s, 5H), 5.09 (s, 2H), 4.56-4.64 (m, 1H), 3.79 (s, 3H), 1.41 (d, J=6.64 Hz, 3H). 13C NMR (200 MHz, CDCl₃) δ (ppm) 171.37, 160.24, 156.39, 156.28, 135.5, 128.5, 128.34, 128.11, 67.81, 53.68, 47.84, 17.65. IR (KBr) ν 4000-3293 (br.), 3035, 2956, 1702, 1689 (br.), 1524, 1456, 1283, 1218, 985, 742, 667 cm⁻¹.

### N—[(S)-2-(N′-Benzyloxycarbonyl-hydrazino)-1-methyl-2-oxo-ethyl]-oxalamic acid (Z-NAlaO-OH

### Example 52

Following the procedure of Example 47 using N—[(S)-2-(N′-Benzyloxycarbonyl-hydrazino)-1-methyl-2-oxo-ethyl]-oxalamic acid t-butyl ester (Example 50) 2.2 g (99%) of desired compound was obtained as a white solid; mp. 173-174° C. 1H NMR (200 MHz, DMSO-d₆) δ (ppm) 9.7 (s, 1H), 9.04 (s, 1H), 8.52 (d, J=7.62 Hz, 1H), 7.14 (s, 5H), 4.86 (s, 2H), 4.08-4.15 (m, 1H), 1.1 (d, J=6.46 Hz, 3H). 13C NMR (200 MHz, DMSO-d₆) δ (ppm) 172.27, 162.81, 159.04, 157.05, 137.66, 129.5, 129.11, 128.93, 67.03, 48.36, 18.82.

### [N′-(9H-Fluoren-9-ylmethoxycarbonyl)-hydrazino]-oxo-acetic acid t-butyl ester

### Example 53

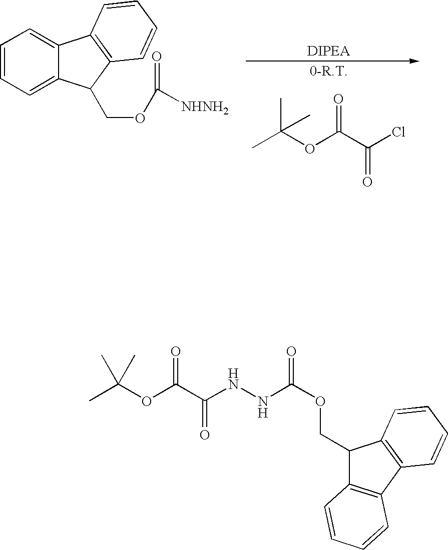

To a mixture of Fmoc-hydrazine (0.100 mg, 0.39 mmol), DIPEA (0.08 mL, 0.43 mmol) and methylenechloride (10 mL) was added t-Bu-oxalylchloride (0.06 mL, 0.39) slowly with stirring on ice-bath under argon. The reaction mixture was stirred further at rt for 15 min. The reaction mixture was then washed with 1N HCL (5 mL), followed by 10% NaHCO₃ (5 mL) and brine (5 mL), dried (Na₂SO₄) and concentrated to give 140 mg of crude compound which was purified by column chromatography to give 94 mg (62%) of a white solid; mp. 79-80° C.; Rf=0.45 (2:1 Pentane/EtOAc). 1H NMR (200 MHz, CDCl₃) δ (ppm) 8.94 (br s, 1H), 7.75 (d, J=7.42 Hz, 2H), 7.58 (d, J=7.24 Hz, 2H), 7.26-7.43 (m, 5H), 4.44 (d, J=7.22 Hz, 2H), 4.23 (t, J=7.14 Hz, 1H), 1.57 (m, 12H). 13C NMR (200 MHz, CDCl₃).6(ppm) 157.78, 156.38, 155.51, 143.33, 141.25, 127.84, 127.16, 125.1, 120.01, 85.69, 68.3, 46.79, 27.70.

### [N′-(9H-Fluoren-9-ylmethoxycarbonyl)-hydrazino]-oxo-acetic acid

### Example 54

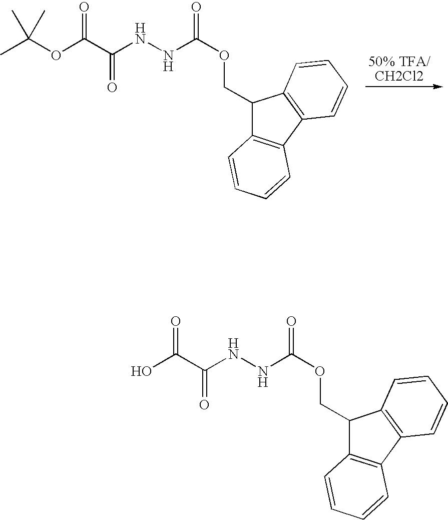

Following the procedure of Example 47 using N′-(9H-Fluoren-9-ylmethoxycarbonyl)-hydrazino]-oxo-acetic acid t-butyl ester (Example 53) 76 mg (100%) of product was obtained as a white solid; mp. 178-179° C. 1H NMR (200 MHz, DMSO-d₆) δ (ppm) 10.64 (s, 1H), 9.5 (s, 1H), 7.88 (d, J=7.24 Hz, 2H), 7.72 (d, J=6.64 Hz, 2H), 7.32-7.45 (m, 4H), 4.22-4.35 (m, 3H). 13C NMR (200 MHz, DMSO-d₆) δ (ppm) 161.19, 158.19, 155.43, 143.51, 140.67, 127.67, 127.06, 125.18, 120.09, 66.19, 46.38.

### N′-((S)-2-t-Butoxycarbonylamino-3-phenyl-propionyl)-N-methyl-hydrazine carboxylic acid 9H-fluoren-9-ylmethyl ester

### Example 55

Following the procedure of Example 2 using Boc-L-phenylalanine and 1-Fmoc-1

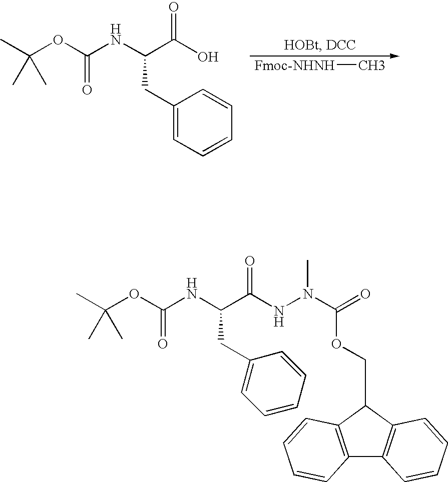

-methylhydrazine 794 mg (81%) of product was obtained as a white solid; mp. 81-82° C. Rf=0.68 (1:1 EtOAc/Pentane); [α]_{D}−12.0 (c 0.5, MeOH). 1H NMR (200 MHz, CDCl₃) δ (ppm) 8.48 (bs, 1H), 7.75 (d, J=7.22 Hz, 2H), 7.56 (d, J=7.24 Hz, 2H), 7.23-7.43 (m, 9H), 5.24 (bs, 1H), 4.19-4.41 (m, 4H), 3.07 (bs, 5H), 1.37 (s, 9H). 13C NMR (200 MHz, CDCl₃) δ (ppm) 170.06, 155.9, 155.56, 143.66, 141.23, 136.35, 129.42, 128.58, 127.73, 127.14, 126.96, 125.06, 119.95, 80.5, 68.31, 54.03, 46.96, 38.32, 37.32, 28.21. IR (KBr) ν 4000-3289 (br.), 3064, 3029, 2977, 1681 (br.), 1478, 1496, 1451, 1392, 1366, 1348, 1249, 1165, 758, 740 cm⁻¹.

### N′-((S)-2-t-Butoxycarbonylamino-3-phenyl-propionyl)-N′-methyl-hydrazine carboxylic acid 9H-fluoren-9-ylmethyl ester

### Example 56

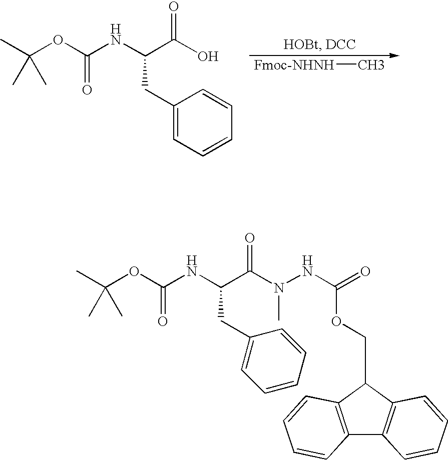

Following the procedure of Example 2 using Boc-L-phenylalanine and 1-Fmoc-2-methylhydrazine 632 mg (65%) of product was obtained as a white solid; mp. 108-109° C.; [α]_{D}+23.6 (c 0.5, MeOH). 1H NMR (200 MHz, CDCl₃) 6(ppm) 7.79 (d, J=7.24 Hz, 2H), 7.17-7.74 (m, 12H), 5.3 (bs, 1H), 4.91 (bs, 1H), 4.49 (bs, 2H), 4.21 (m, 1H), 2.66-3.31 (m, 5H), 1.39 (s, 9H). 13C NMR (200 MHz, CDCl₃) δ (ppm) 173.92, 155.35, 154.71, 143.32, 141.36, 136.53, 129.35, 128.47, 127.9, 127.17, 126.9, 124.91, 120.07, 79.88, 67.77, 51.23, 46.94, 35.64, 28.27. IR (KBr) ν 4000-3383, 3224, 3064, 3004, 2977, 2929, 1742, 1697, 1647, 1516, 1451, 1391, 1366, 1248, 1170, 1115, 1083, 1048, 756, 740 cm⁻¹.

### N-{(S)-1-Benzyl-2-[N′-(9H-fluoren-9-ylmethoxycarbonyl)-N′-methyl-hydrazino]-2-oxo-ethyl}-oxalamic acid t-butyl ester

### Example 57

To the solution of N′-((S)-2-t-Butoxycarbonylamino-3-phenyl-propionyl)-N-methyl-hydrazine

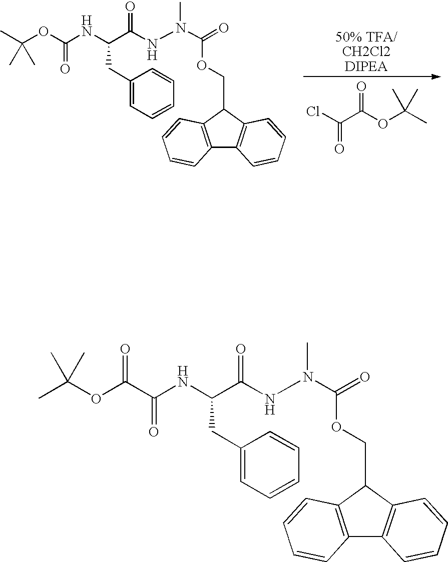

carboxylic acid 9H-fluoren-9-ylmethyl ester (Example 55) (687 mg, 1.33 mmol) in DCM (10 mL), TFA (10 mL) was added slowly with stirring at 0° C., the reaction mixture was stirred overnight at rt. Solvent was evaporated and was well dried under high vacuum. The residue obtained was dissolved in dry DCM (30 mL), and DIPEA (0.7 mL, 4.0 mmol) was added to the reaction mixture, followed by t-butyl oxalylchloride (219 mg, 1.33 mmol) at 0° C. under argon. The reaction mixture was further stirred at rt for 30 min. The mixture was washed with 1N HCl (15 mL) followed by 10% NaHCO₃ (15 mL) and brine (15 mL). The organic phase was dried (Na₂SO₄) and concentrated to give 770 mg of crude product which was purified by column chromatography to give 635 mg (87%) of product as a white powder. Rf=0.2 (1:2 EtOAc/Pentane). 1H NMR (200 MHz, CDCl₃) δ (ppm) 8.64 (s, 1H), 7.62 (d, J=7.04 Hz, 3H), 7.43 (d, J=7.04 Hz, 2H), 7.13-7.3 (m, 9H), 4.78 (q, J=7.24 Hz, 1H), 3.97-4.27 (m, 3H), 3.03 (bs, 2H), 2.93 (s, 3H), 1.33 (s, 9H). 13C NMR (200 MHz, CDCl₃) δ (ppm) 169.0, 158.29, 157.49, 155.82, 143.61, 141.2, 135.7, 129.42, 128.69, 127.75, 127.23, 127.12, 125.05, 119.95, 85.01, 68.44, 53.08, 46.87, 38.11, 37.51, 27.56.

### N-{(S)-1-Benzyl-2-[N′-(9H-fluoren-9-ylmethoxycarbonyl)-N-methyl-hydrazino]-2-oxo-ethyl}-oxalamic acid t-butyl ester

### Example 58

Following the procedure of Example 57 using N′-((S)-2-t-Butoxycarbonylamino-3

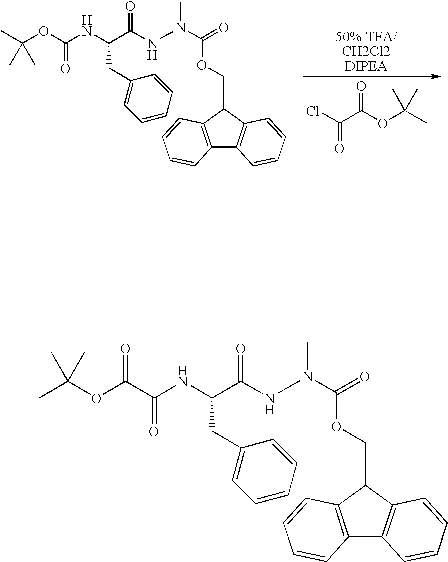

-phenyl-propionyl)-N′-methyl-hydrazine carboxylic acid 9H-fluoren-9-ylmethyl ester (Example 56) 463 mg (83%) of product was obtained as a white powder. Rf=0.26 (1:2 EtOAc/Pentane). 1H NMR (200 MHz, CDCl₃) δ (ppm) 7.68 (d, J=7.22 Hz, 2H), 7.07-7.6 (m, 13H), 4.98 (bs, 1H), 4.39 (bs, 2H), 4.11 (bs, 1H), 2.97 (bs, 5H), 1.43 (s, 9H). 13C NMR (200 MHz, CDCl₃) δ (ppm) 172.37, 158.6, 157.07, 154.76, 143.29, 141.35, 135.75, 129.32, 128.61, 127.91, 127.23, 127.16, 124.91, 120.07, 84, 78, 67.75, 50.73, 46.93, 35.69, 27.68.

### N′-{(S)-2-[((S)-1-Dimethylcarbamoyl-ethylaminooxalyl)-amino]-3-phenylpropionyl}-hydrazine carboxylic acid t-butyl ester

### Example 59

Following the procedure of Example 2 using N—[(S)-1-Benzyl-2-(N′-t-butoxycarbonyl-hydrazino)-2-oxo-ethyl]-oxalamic

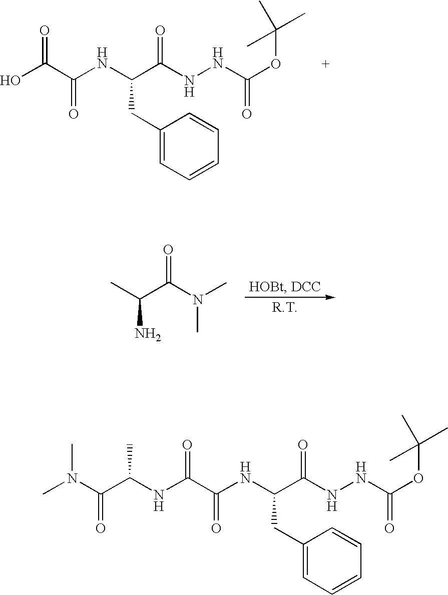

acid (Example 3) and (S)-2-Amino-N,N-dimethyl-propionamide 122 mg (35%) of product was obtained as off-white solid; mp 104-107° C.; [α]_{D}−(c 0.5, MeOH). 1H NMR (200 MHz, CDCl₃) δ (ppm) 8.6 (br s, 1H), 8.25-8.29 (br, 2H), 7.14-7.2 (m, 5H), 6.59 (br s, 1H), 4.68-4.8 (m, 2H), 3.29 (dd, J=5.28 Hz, 1H), 2.93-3.07 (m, 4H), 2.88 (s, 3H), 1.35 (s, 9H), 1.26 (d, J=6.26 Hz, 3H). 13C NMR (200 MHz, CDCl₃) δ (ppm) 171.17, 169.77, 159.60, 158.42, 155.18, 136.39, 129.25, 128.60, 126.93, 81.75, 53.11, 45.66, 37.29, 36.98, 35.84, 28.08, 17.88. IR (KBr) ν 4000-3292 (br.), 3030, 2980, 2935, 1674 (br.), 1641, 1498 cm⁻¹.

### {(S)-1-Benzyl-2-[N′-((S)-2-t-butoxycarbonylamino-propionyl)-hydrazino]-2-oxo-ethyl}-carbamic acid benzyl ester

### Example 60

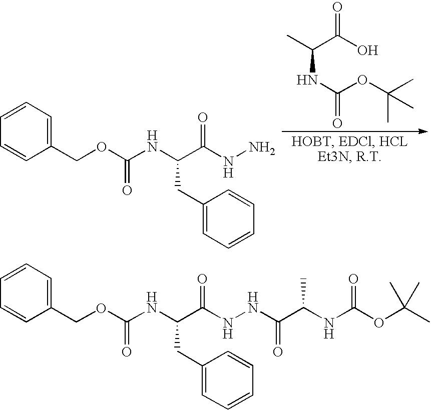

Boc-L-Ala (1.81 g, 9.57 mmol) was dissolved in DCM (30 mL) and HOBt (1.36 g, 10.05 mmol) was added followed by (1.93 g, 10.05 mmol) EDCI.HCl. To this solution N-benzyloxycarbonyl-L-phenylalanine hydrazide (3.0 g, 9.57 mmol) was added followed by triethylamine (1.47 mL, 10.53 mmol). The reaction mixture was further stirred at rt overnight. DCM removed by rotary evaporation and the residue obtained was dissolved in EtOAc (50 mL). The EtOAc solution was washed with 1N HCl (15 mL), 10% NaHCO₃ (15 mL), brine (15 mL). The EtOAc was heated to dissolve the precipitated compound and pet ether (10 mL) was added. The solution was allowed to stand for overnight. The crystallized compound was filtered to give 3.7 g (79%) of product as a white crystalline solid; mp. 197° C.-198° C. 1H NMR (200 MHz, DMSO-d₆) δ (ppm) 10.18 (br s, 1H), 9.93 (br s, 1H), 7.54 (d, J=8.4 Hz, 1H), 7.17-7.35 (m, 10H), 6.96 (d, J=7.62 Hz, 1H), 4.92 (s, 2H), 4.25-4.36 (m, 1H), 4.0-4.08 (m, 1H), 2.69-3.09 (m, 2H), 1.38 (s, 9H), 1.23 (d, J=7.24 Hz, 3H).

### {(S)-1-[N′-((S)-2-Amino-3-phenyl-propionyl)-hydrazinocarbonyl]-ethyl}-carbamic acid t-butyl ester

### Example 61

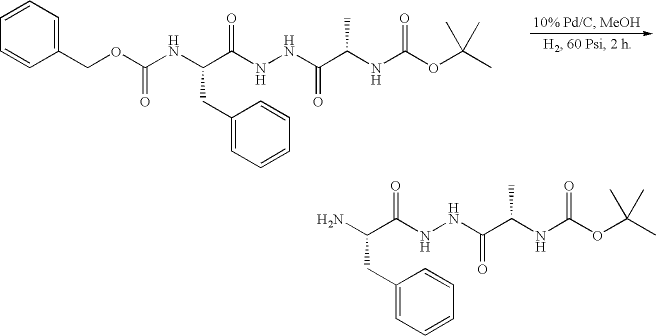

In a Parr apparatus a solution of {(S)-1-Benzyl-2-[N′-((S)-2-t-butoxycarbonylamino-propionyl)-hydrazino]-2-oxo-ethyl}-carbamic acid benzyl ester (Example 60) (835 mg, 1.72 mmol) in MeOH (15 mL) was hydrogenated using 10% Pd/C (125 mg, 15% w/w) at 60 psi for 2 hr at rt. The catalyst was filtered using sintered glass funnel and the filtrate was evaporated to give 568 mg (94%) of the product as a off-white solid; mp. 76-77° C. ¹H NMR (200 MHz, DMSO-d₆) δ (ppm) 7.26 (br s, 5H), 6.94 (d, J=9.78 Hz, 1H), 4.02-4.09 (m, 1H), 3.46-3.53 (m, 1H), 2.93-3.02 (m, 1H), 2.57-2.67 (m, 1H), 1.38 (s, 9H), 1.21 (d, J=5.68 Hz, 3H).

### N-{(S)-1-Benzyl-2-[N′-((S)-2-t-butoxycarbonylamino-propionyl)-hydrazino]-2-oxo-ethyl}-oxalamic acid methyl ester (Boc-Ala-NPheO-OMe

### Example 62

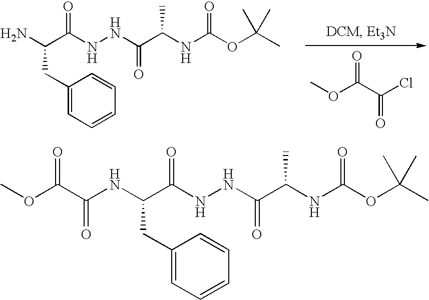

Triethylamine (1.56 mL, 11.13 mmol) was added to a solution of {(S)-1-[N′-((S)-2-Amino-3-phenyl-propionyl)-hydrazinocarbonyl]-ethyl}-carbamic acid t-butyl ester (Example 61) (2.6 g, 7.42 mmol) in dry DCM (10 mL) at 0° under argon followed by methyl oxalylchloride (0.72 mL, 7.79 mmol) and the reaction mixture was stirred for 30 min at 0° and at rt for 1.5 h. The mixture was diluted with EtOAc (50 mL) and washed with 1N HCl (10 mL) followed by 10% NaHCO₃ (10 mL) and brine (10 mL). The EtOAc layer was dried (Na₂SO₄) and concentrated to give 3.2 g of crude compound, which was crystallized from EtOAc-pet ether to give 2.92 g (90%) of a white crystalline solid; mp. 134-135° C. 1H NMR (200 MHz, DMSO-d₆) [symbol] (ppm) 10.21 (s, 1H), 9.9 (s, 1H), 8.96 (d, J=8.6 Hz, 1H), 7.16-7.29 (m, 5H), 6.97 (d, J=7.64 Hz, 1H), 4.54-4.66 (m, 1H), 4.0-4.08 (m, 1H), 3.74 (s, 3H), 2.94-3.18 (m, 2H), 1.38 (s, 9H), 1.22 (d, J=7.64 Hz, 3H).

### N-{(S)-1-Benzyl-2-[N′-((S)-2-t-butoxycarbonylamino-propionyl)-hydrazino]-2-oxo-ethyl}-oxalamic acid (Boc-Ala-NPheO-OH

### Example 63

Following the procedure of Example 3 using N—{(S)-1-Benzyl-2-[N′-((S)-2-t-butoxycarbonylamino-propionyl)-hydrazino]-2-oxo-ethyl}-oxalamic

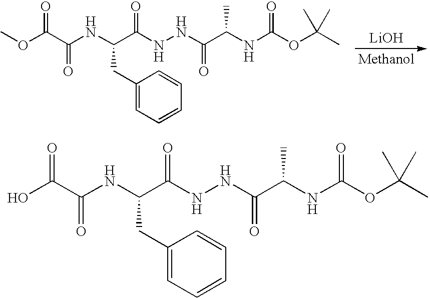

acid methyl ester (Example 62) 2.25 g (79%) of a white crystalline solid; mp 162-163° C. 1H NMR (200 MHz, DMSO-d₆) δ (ppm) 10.21 (s, 1H), 9.90 (s, 1H), 8.69 (d, J=10.16 Hz, 1H), 7.15-7.27 (m, 5H), 6.97 (d, J=7.24 Hz, 1H), 4.51-4.62 (m, 1H), 3.99-4.07 (m, 1H), 2.93-3.16 (m, 2H), 1.36 (s, 9H), 1.21 (d, J=7.24 Hz, 3H). 13C NMR (200 MHz, DMSO-d₆) δ (ppm) 171.79, 168.99, 161.44, 158.11, 154.92, 137.47, 129.1, 128.05, 126.35, 77.90, 53.07, 48.15, 36.86, 28.15, 18.14. IR (KBr) ν 4000-3331, 3258, 3032, 2980, 2937, 1765, 1686, 1616, 1529, 1481 cm⁻¹.

### [(S)-1-(N′-{(S)-2-[((S)-1-Dimethylcarbamoyl-ethylaminooxalyl)-amino]-3-phenyl-propionyl}-hydrazinocarbonyl)-ethyl]-carbamic acid t-butyl ester (Boc-Ala-NPheO-Ala-NMe2

### Example 64

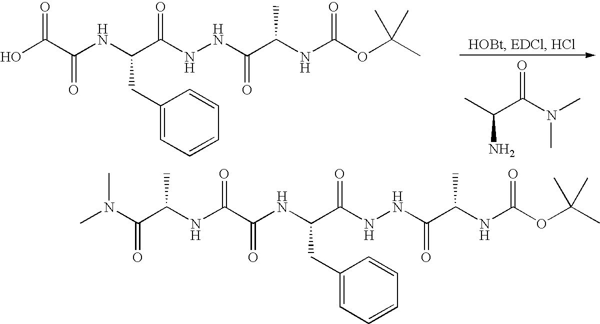

Following the procedure of Example 60 using N-{(S)-1-Benzyl-2-[N′-((S)-2-t-butoxycarbonylamino-propionyl)-hydrazino]-2-oxo-ethyl}-oxalamic acid (Example 63) and (s)-2-Amino-N,N-dimethyl-propionamide desired product was obtained as a crystalline solid; mp. 199° C. 1H NMR (200 MHz, DMSO-d₆) δ (ppm) 10.24 (s, 1H), 9.92 (s, 1H), 8.57 (d, J=8.4 Hz, 1H), 8.37 (d, J=7.64 Hz, 1H), 7.15-7.28 (m, 5H), 6.97 (d, J=7.24 Hz, 1H), 4.56-4.72 (m, 2H), 3.99-4.07 (m, 1H), 3.05-3.1 (m, 2H), 2.99 (s, 3H), 2.82 (s, 3H), 1.37 (s, 9H), 1.18-1.23 (m, 6H). 13C NMR (200 MHz, DMSO-d₆) δ (ppm) 171.8, 170.57, 168.89, 159.19, 158.09, 154.94, 137.25, 129.14, 128.08, 126.44, 77.92, 53.04, 48.14, 45.22, 37.02, 36.35, 35.21, 28.15, 18.11, 17.22. IR (KBr) ν 4000-3353, 3280, 3030, 2983, 2936, 1718, 1686, 1656, 1627, 1516 cm⁻¹.

### Boc-Ala-NPheO-O-Merrifield

### Example 65

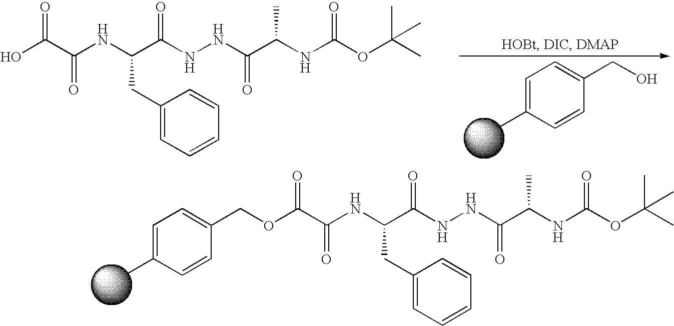

The hydroxymethyl resin (100 mg, 0.104 mmol) was suspended in 9:1 v/v CH₂Cl₂/DMF (1 mL). In separate flask HOBt (42.16 mg, 0.31 mmol) was added to the solution of N-{(S)-1-Benzyl-2-[N′-((S)-2-t-butoxycarbonylamino-propionyl)-hydrazino]-2-oxo-ethyl}-oxalamic acid (Example 64) (131.8 mg, 0.31 mmol) in minimum amount of DMF. The mixture was stirred until the HOBt get dissolved and this solution was added to the resin. DIC (0.05 mL, 0.31 mmol) was then added to the reaction mixture followed by the solution of DMAP (13 mg, 0.11 mmol) in minimum amount of DMF. The reaction mixture was agitated overnight at rt with mechanical shaker under argon. Acetic anhydride and pyridine (2 equivalents relative to the resin) were added to the reaction mixture and agitated for an additional 30 min at rt to end-cap any unreacted hydroxyl groups on the resin. Resin was filtered in a fine sintered glass funnel and washed with DMF (3×5 mL), DCM (3×5 mL), MeOH (3×5 mL) and dried in vacuum to a constant weight.

### Ala-NPheO-O-Merrifield

### Example 66

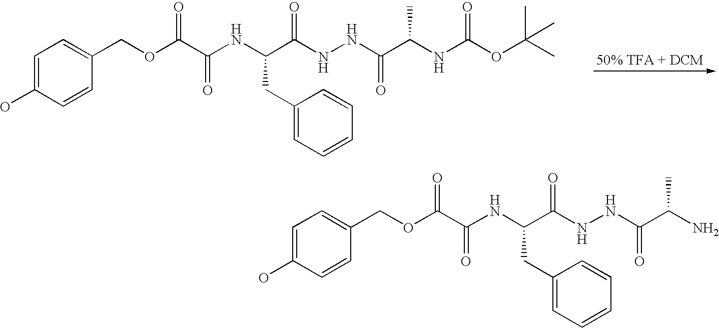

The suspension of resin (Example 65) (0.104 mmol) in 50% (v/v) TFA/DCM (1 mL) was agitated at rt using mechanical shaker for 30 min. Resin was filtered in a fine sintered glass funnel and washed with DCM (3×5 mL) followed by 5% (v/v) DIPEA (2 mL) to remove TFA and dried in vacuum to a constant weight.

### Boc-Phe-Ala-NPheO-O-Merrifield

### Example 67

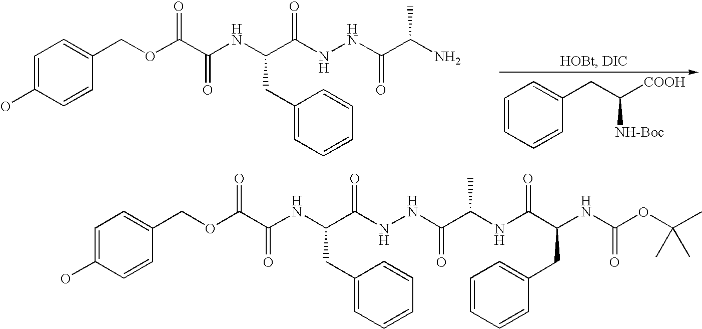

The resin (Example 66) (0.104 mmol) was suspended in 9:1 v/v CH₂Cl₂/DMF (1 mL). In separate flask HOBt (42.16 mg, 0.31 mmol) was added to the solution of Boc-L-Phenylalanine (82.8 mg, 0.31 mmol) in minimum amount of DMF. The mixture was stirred until the HOBt get dissolved and this solution was added to the resin. DIC (0.05 mL, 0.31 mmol) was then added and the reaction mixture was agitated overnight at rt with mechanical shaker under argon.

Resin was filtered in a fine sintered glass funnel and washed with DMF (3×5 mL), DCM (3×5 mL), MeOH (3×5 mL) and dried in vacuum to a constant weight. N—((S)-1-Benzyl-2-{N′—[(S)-2-((S)-2-t-butoxycarbonylamino-3-phenyl-propionylamino)-propionyl]-hydrazino}-2-oxo-ethyl)-oxalamic acid methyl ester (Boc-Phe-Ala-NPheO-OMe

### Example 68

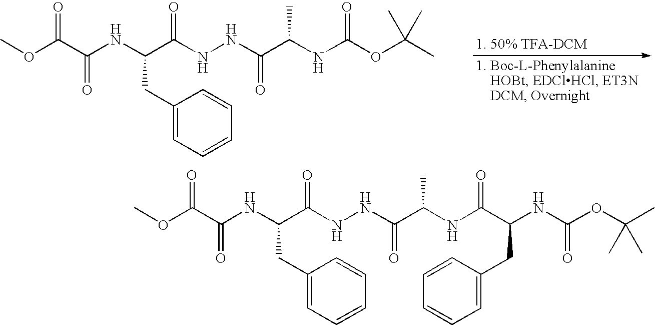

To the solution of N-{(S)-1-Benzyl-2-[N′-((S)-2-t-butoxycarbonylamino-propionyl)-hydrazino]-2-oxo-ethyl}-oxalamic acid methyl ester (Example 62) (178 mg, 0.41 mmol) in DCM (5 mL), TFA (5 mL) was added slowly with stirring at 0° C. The reaction mixture was stirred at rt for 30 min. DCM and TFA was evaporated and reaction mixture was well dried under high vacuum, the residue obtained was dissolved in dry DCM (5 mL) and was added to the another flask containing the solution of Boc-L-Phenylalanine (113.6 mg, 0.43 mmol), HOBt (57.86 mg, 0.43 mmol), EDCI.HCl (82.09 mg, 0.43 mmol) in DCM (5 mL). Triethylamine (0.17 mL 1.22 mmol) was added and the reaction mixture was stirred at rt for overnight. DCM removed by rotary evaporation and the residue obtained was dissolved in EtOAc (10 mL). The EtOAc solution was washed with 1N HCl (5 mL), 10% NaHCO₃ (5 mL), brine (5 mL), dried (Na₂SO₄) and concentrated to give 95 mg (39%) of desired product as a white solid. 1H NMR (200 MHz, DMSO-d₆) δ (ppm) 10.29 (brs, 1H), 10.05 (brs, 1H), 9.04 (d, J=9.78 Hz, 1H), 8.17 (d, J=8.42 Hz, 1H), 7.27-7.35 (m, 10H), 6.95 (d, J=9.78 Hz, 1H), 4.61-4.72 (m, 1H), 4.41-4.49 (m, 1H), 4.07-4.32 (m, 1H), 3.8 (s, 3H), 2.94-3.25 (m, 3H), 2.68-2.82 (m, 1H), 1.34 (m, 12H).

### N—((S)-1-Benzyl-2-{N′—[(S)-2-((S)-2-t-butoxycarbonylamino-3-phenyl-propionylamino)-propionyl]-hydrazino}-2-oxo-ethyl)-oxalamic acid (Boc-Phe-Ala-NPheO-OH

### Example 69

a) via solid phase synthesis: To the suspension of resin (Example 67) (0.104 mmol) in 1:4 v/v MeOH-THF (2 mL) was added NaOCH₃ (0.6 mg, 0.01 mmol). The reaction mixture

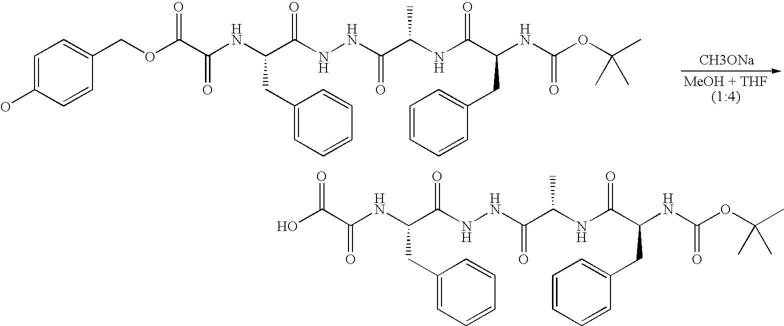

was stirred at 70° C. for 18 h. Water (3 drops) was added and it was stirred for additional 30 min at 70° C. The reaction mixture was filtered through fine glass sintered funnel, washed with MeOH (5 mL). The filtrate was concentrated to remove MeOH, residue obtained was dissolved in 10% NaHCO₃ aqueous solution (5 mL) and washed with ether (2×5 mL). The aqueous layer was cooled and acidified with 1N HCL and extracted with EtOAc (3×5 mL). The combined organic layer was washed with brine (2×5 mL), dried (Na₂SO₄) and concentrated to give 9 mg (15%) of an off-white solid.

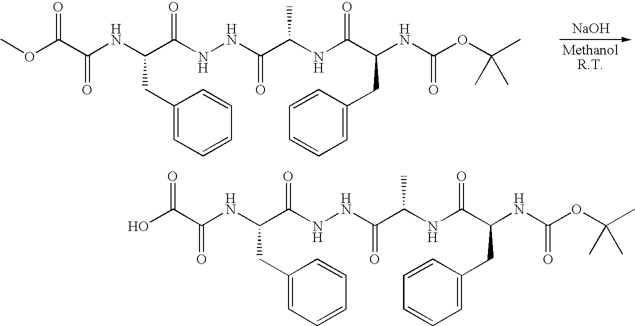

b) via liquid phase synthesis: To the solution of N—((S)-1-Benzyl-2-{N′—[(S)-2-((S)-2-t-butoxycarbonylamino-3-phenyl-propionylamino)-propionyl]-hydrazino}-2-oxo-ethyl)-oxalamic acid methyl ester (Example 68) (85 mg, 0.15 mmol) in MeOH (2 mL) was added NaOCH₃ (6 mg, 0.15 mmol), and stirred at rt for 1 h. MeOH was evaporated and the residue obtained was dissolved in water (10 mL), washed with ether (2×5 mL). The aqueous layer was cooled in ice bath, acidified (pH=4) with 1N HCl and extracted with EtOAc (3×10 mL). The combined organic layer was washed with brine (10 mL), dried (Na₂SO₄) and concentrated to give 26 mg (31%) of desired compound as a off-white crystalline solid; mp. 155-156° C. 1H NMR (200 MHz, DMSO-d₆) δ (ppm) 10.28 (br s, 1H), 10.04 (br s, 1H), 8.79 (br s, 1H), 8.15 (br s, 1H), 6.5-7.27 (m, 10H), 4.39 (m, 2H), 2.76-3.2 (m, 5H), 1.29 (br s, 12H). IR (KBr) ν 4000-3299(broad), 3030, 2979, 2933, 1702, 1696, 1687, 1674, 1652, 1508 cm⁻¹.

### [(S)-2-Methyl-1-((S)-3-methyl-1-methylcarbamoyl-butylcarbamoyl)-butyl]-carbamic acid t-butyl ester

### Example 70

### Step 1

### ((S)-3-Methyl-1-methylcarbamoyl-butyl)-carbamic acid t-butyl ester

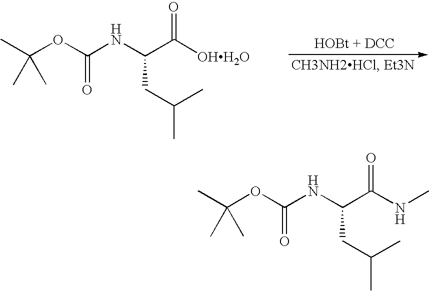

To the solution of Boc-L-Leu monohydrate (2.0 g, 8.02 mmol) in DCM (30 mL), HOBt (1.14 g, 8.42 mmol) was added followed by DCC (1.74 g, 8.42 mmol). To this solution a suspension of methylamine HCl (596 mg, 8.82 mmol) and triethylamine (1.68 mL, 12.03 mmol) in DCM (15 mL) was added and the mixture was stirred at rt for 24 h. The precipitated DCHU was removed by filtration and the filtrate was evaporated. Additional DCHU was removed by subsequent trituration with cold EtOAc and filtration. The EtOAc solution was washed with 1N HCl (2×15 mL), 10% NaHCO₃ (15 mL), brine (15 mL), dried (Na₂SO₄) and concentrated to give 1.5 g (76%) of a white solid. 1H NMR (200 MHz, CDCl₃) δ (ppm) 6.73 (br s, 1H), 5.2 (br s, 1H), 4.07-4.1 (m, 1H), 2.71 (d, J=4.7 Hz, 3H), 1.36-1.67 (m, 3H), 1.3 (s, 9H), 0.86 (dd, J=2.34, 2.14 Hz, 6H). 13C NMR (200 MHz, CDCl₃) δ (ppm) 173.46, 155.81, 79.76, 53, 41.62, 28.26, 26.03, 24.68, 22.88, 21.91.

### Step 2:

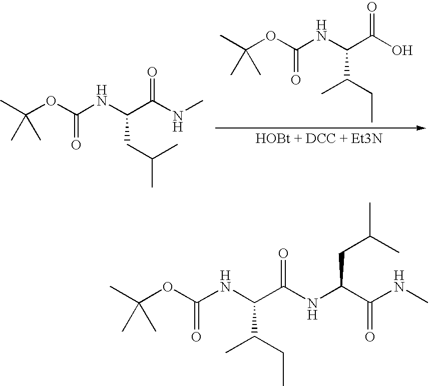

To the solution of ((S)-3-Methyl-1-methylcarbamoyl-butyl)-carbamic acid t-butyl ester (1.47 g, 6.02 mmol) in DCM (15 mL), TFA (15 mL) was added slowly with stirring at 0° C. The reaction mixture was stirred at rt for 30 min. Solvent was evaporated and well dried under high vacuum, the residue obtained was dissolved in DCM (30 mL). To this solution was added Boc-isoleucine (compound 71) (1.53 g, 6.62 mmol), HOBt (0.89 g, 6.62 mmol), DCC (1.74 g, 6.62 mmol) followed by triethylamine (2.53 mL, 18.05 mmol) and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (2×15 mL), 10% NaHCO₃ (15 mL), brine (15 mL), dried (Na₂SO₄) and concentrated to give 2.1 g (97%) of desired product as a white solid; mp. 151-152° C. Rf=(1:1 EtOAc/Pentane). 1H NMR (200 MHz, CDCl₃) δ (ppm) 7.19 (br s, 1H), 6.95 (br s, 1H), 5.42 (d, J=8.2 Hz, 1H), 4.37-4.48 (m, 1H), 3.92-4.0 (m, 1H), 2.69 (d, J=4.7 Hz, 3H), 1.36-1.81 (m, 5H), 1.27 (s, 9H), 0.97-1.25 (m, 1H), 0.77-0.86 (m, 12H). 13C NMR (200 MHz, CDCl₃) δ (ppm) 172.51, 172.18, 155.97, 79.74, 59.36, 51.69, 40.44, 37.08, 28.25, 26.03, 24.92, 24.71, 22.79, 21.98, 15.39, 11.22.

### {(S)-1-[(S)-2-Methyl-1-((S)-3-methyl-1-methylcarbamoyl-butylcarbamoyl)-butylcarbamoyl]-2-phenyl-ethyl}-carbamic acid t-butyl ester

### Example 71

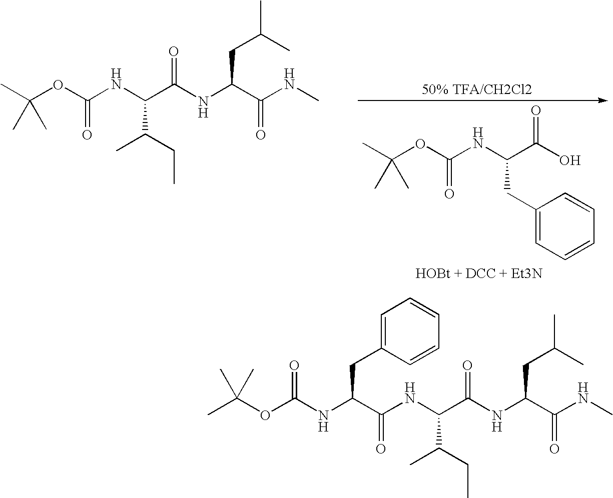

To the solution of [(S)-2-Methyl-1-((S)-3-methyl-1-methylcarbamoyl-butylcarbamoyl)butyl]-carbamic acid t-butyl ester (Example 70) (2.1 g, 6.02 mmol) in DCM (15 mL), TFA (15 mL) was added slowly with stirring at 0° C. The reaction mixture was stirred at rt for 30 min. Solvent was evaporated and was well dried under high vacuum, the residue obtained was dissolved in DCM (30 mL). To this solution was added Boc-Phe (1.53 g, 6.62 mmol), HOBT (0.89 g, 6.62 mmol), DCC (1.74 g, 6.62 mmol) followed by triethylamine (2.53 mL, 18.05 mmol) and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (2×15 mL), 10% NaHCO₃ (15 mL), brine (15 mL), dried (Na₂SO₄) and concentrated to give 2.1 g (97%) of desired product as a white solid; mp. 151-152° C. Rf=(1:1 EtOAc/Pentane). 1H NMR (200 MHz, DMSO-d₆) δ (ppm) 7.98 (d, J=7.82 Hz, 1H), 7.78 (br s, 2H), 7.23 (s, 5H), 6.97 (d, J=7.82 Hz, 1H), 4.19-4.23 (m, 3H), 2.53-2.97 (m, 5H), 1.01-1.72 (m, 15H), 0.79-0.82 (m, 12H).

### Synthesis of a Beta-Turn Peptide Motif Using a Compound of Formula I as Sub-Structure

### Example 72

### Step 1

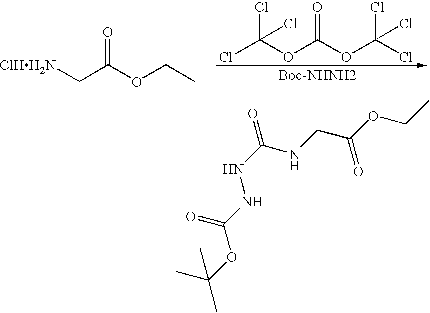

### 2-[[(2-ethoxy-2-oxoethyl)amino]carbonyl]-hydrazinecarboxylic acid, 1,1-dimethylethyl ester (Boc-aza-Gly-OEt)

To the solution of triphosgene (3.4 g, 11.46 mmol) in CH₂Cl₂ (10 mL), a mixture of glycine ethyl ester HCl (4.0 g, 28.66 mmol) and DIEA (14.87 mL, 85.97 mmol) in CH₂Cl₂ (15 mL) was added slowly over a period of 30 min. After a further 15 min of stirring, a solution of Boc-Hydrazine (3.79 g, 28.66 mmol) in CH₂Cl₂ (15 mL) was added in one portion. The reaction mixture was further refluxed for 2 hr, evaporated to dryness, diluted with EtOAc, washed with 10% aq NaHCO₃ and brine, dried over Na₂SO₄ and evaporated to give 5.6 g of crude compound which was purified by column chromatography to give 3.63 g (48%) of product as a sticky mass which after overnight standing at rt gave an off-white solid; mp. 137-138° C. Rf=0.29 (5% MeOH in CH₂Cl₂). 1H NMR (200 MHz, CDCl₃) δ (ppm) 7.2 (bs, 1H), 7.05 (s, 1H), 6.2 (t, J=5.46 Hz, 1H), 4.11 (q, J=7.04 Hz, 2H), 3.92 (d, J=5.66 Hz, 2H), 1.39 (s, 9H), 1.2 (t, J=7.04 Hz, 3H). 13C NMR (200 MHz, CDCl₃) δ (ppm) 171.07, 158.95, 156.43, 81.51, 61.25, 41.79, 28.09, 14.04.

References for this Step:

This compound was prepared as described in Gacel, G. Zajac, J. M. DelayGoyet, P. Dauge, V. Roques, B. P. Investigation of the structural parameters involved in the μ and δ opioid receptor discrimination of linear enkephalin-related peptides. Journal of Medicinal Chemistry (1988), 31(2), 374-83.

### Step 2: Boc-Pro-aza-Gly-OEt

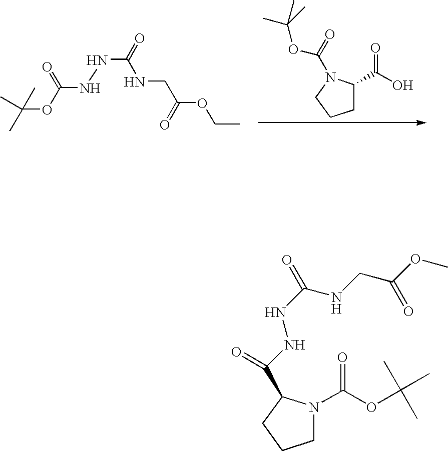

To the solution of Boc-aza-Gly-OEt (3.63 g, 13.89 mmol) in DCM (15 mL), TFA (15 mL) was added slowly with stirring at 0° C., the reaction mixture was stirred at rt for 30 min. Solvent was evaporated and well dried under high vacuum, the residue obtained was dissolved in DCM (15 mL). In another flask Boc-Pro (2.99 g, 13.89 mmol), HOBt (1.97 g, 14.59 mmol) and DCC (3.09 g, 14.59 mmol) were dissolved in DCM (15 mL) and stirred for 15 min at rt, to this a mixture of above TFA salt and triethylamine (4.77 mL, 34.73 mmol) in DCM (15 mL) was added and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (2×15 mL), 10% NaHCO₃ (15 mL), brine (15 mL), dried (Na₂SO₄) and concentrated to give 5.2 g of crude compound which was purified by column chromatography to give 3.2 g (48%) of product as a brown solid; mp. 58-59° C. 1H NMR (200 MHz, CDCl₃) δ (ppm) 9.03 (bs, 1H), 7.47 (bs, 1H), 6.65 (bs, 1H), 3.76-4.2 (m, 5H), 3.35-3.49 (m, 2H), 1.67-2.08 (m, 4H), 1.35 (s, 9H), 1.19 (t, J=7.04 Hz, 3H). 13C NMR (200 MHz, CDCl₃) δ (ppm) 173.1, 172.7, 171.07, 155.48, 80.56, 61.19, 59.02, 47.13, 41.78, 29.38, 28.33, 24.62, 14.07.

### EtO-Gly-NProO-O-tBu (Step 3 and

### Example 73

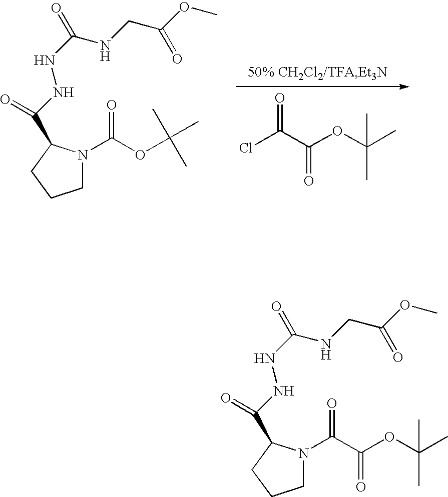

To the solution of Boc-Pro-aza-Gly-OEt (Example 73) (2.63 g, 7.34 mmol) in DCM (15 mL), TFA (15 mL) was added slowly with stirring at 0° C., the reaction mixture was stirred overnight at rt. Solvent was evaporated and was well dried under high vacuum. The residue obtained was dissolved in DCM (30 mL), to that was added slowly with stirring triethylamine (4.1 mL, 29.35 mmol) at 0° C., followed by t-butyl oxalylchloride (1.27 g, 7.71 mmol) and the reaction mixture was further stirred under argon at rt for 30 min. The solvent was evaporated and the crude compound obtained was purified by column chromatography to give 2.2 g (77%) of a sticky mass. 1H NMR (200 MHz, CDCl₃) δ (ppm) 9.1 (bs, 1H), 7.54 (bs, 1H), 6.46 (t, J=5.48 Hz, 1H), 4.38 (t, J=5.48 Hz, 1H), 4.09 (q, J=7.04 Hz, 2H) 3.58-4.0 (m, 4H), 1.73-2.16 (m, 4H), 1.47 (s, 9H), 1.18 (t, J=7.04 Hz, 3H). 13C NMR (200 MHz, CDCl₃) δ (ppm) 171.26, 171.16, 160.59, 160.02, 158.76, 84.6, 61.29, 59.08, 48.57, 41.85, 28.62, 27.83, 27.68, 25.1, 14.05.

### Boc-aza-Gly-N(Me)₂ (Step 4 and

### Example 74

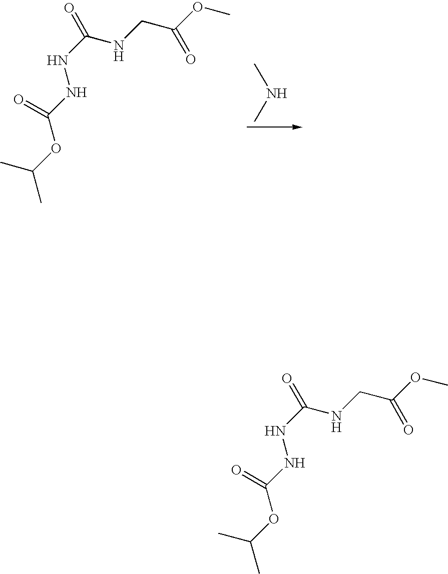

The solution of Boc-aza-Gly-OEt (73 step-1) (3.8 g, 14.54 mmol) in 33% solution of dimethylamine in EtOH (20 mL) was stirred overnight at rt. Solvent was evaporated and was well dried under high vacuum to give 3.78 g (100%) of an off-white solid; mp. 71-72° C. Rf=0.24 (5% MeOH in CH₂Cl₂). 1H NMR (200 MHz, CDCl₃) δ (ppm) 7.35 (bs, 1H), 7.07 (s, 1H), 6.48 (t, J=4.3 Hz, 1H), 4.0 (d, J=4.3 Hz, 2H), 2.92 (s, 3H), 2.89 (s, 3H), 1.38 (s, 9H). 13C NMR (200 MHz, CDCl₃) δ (ppm) 169.15, 158.74, 156.33, 81.08, 41.8, 36.01, 35.64, 28.13.

### Boc-Pro-aza-Gly-N(Me)₂ (Step 5 and

### Example 75

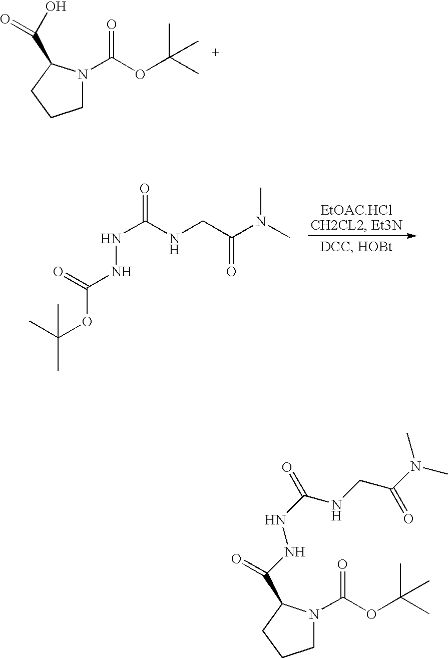

To the solution of Boc-aza-Gly-N(Me)₂ (Example 75) (1.87 g, 7.2 mmol) in EtOAc (10 mL), EtOAc saturated with HCl (10 mL) was added slowly with stirring at 0° C. The reaction mixture was stirred at rt for 30 min. Solvent was evaporated and the reaction mixture was dried under high vacuum. In another flask Boc-Pro (1.55 g, 7.2 mmol), HOBt (1.02 g, 7.56 mmol) and DCC (1.56 g, 7.56 mmol) were dissolved in DCM (15 mL) and stirred for 15 min at rt, to this a mixture of above HCl salt and triethylamine (3.0 mL, 21.6 mmol) in DCM (15 mL) was added and the mixture was stirred overnight at rt. The precipitated DCHU was removed by filtration and the filtrate was evaporated. The additional DCHU was removed by subsequent trituration with cold EtOAc and filtration. The EtOAc solution was concentrated. The crude compound was purified by column chromatography to give 2.1 g (81%) of product as a white solid: mp. 74-75° C. Rf=0.51 (10% MeOH/CH₂Cl₂). 1H NMR (200 MHz, CDCl₃) δ (ppm) 8.89 (bs, 1H), 7.75 (bs, 1H), 6.56 (bs, 1H), 3.87-4.22 (m, 3H), 3.41 (m, 2H), 2.91 (s, 3H), 2.88 (s, 3H), 1.68-2.1 (m, 4H), 1.36 (s, 9H). 13C NMR (200 MHz, CDCl₃) δ (ppm) 172.46, 169.06, 158.28, 155.33, 80.33, 58.72, 53.44, 47.07, 41.83, 36.05, 35.71, 28.33, 24.49.

### t-BuO-Oxalyl-Pro-aza-Gly-(NMe)₂ ((Me)₂N-Gly-NProO-OtBu

### Example 76

To the solution of Boc-Pro-aza-Gly-N(Me)₂ (Example 76) (0.72 g, 2.02 mmol) in DCM

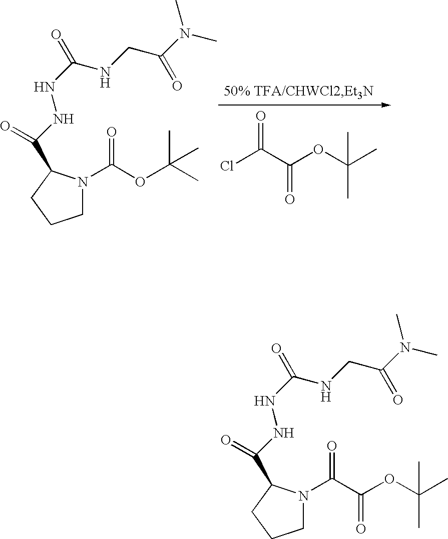

(10 mL), TFA (10 mL) was added slowly with stirring at 0° C., the reaction mixture was further stirred under argon at rt for 30 min. Solvent was evaporated and dried under high vacuum. The residue obtained was dissolved in DCM (25 mL), to that was added slowly with stirring triethylamine (0.85 mL, 6.04 mmol) at 0° C., followed by t-butyl oxalylchloride (0.33 g, 2.02 mmol) and the reaction mixture was further stirred under argon at rt for 30 min. The solvent was evaporated and the crude compound obtained was purified by column chromatography to give 250 mg (32%) of product as white solid. mp. 172-173° C. Rf=0.36 (10% MeOH/CH₂Cl₂). 1H NMR (200 MHz, DMSO-d₆) δ (ppm) 9.78 (bs, 1H), 8.15 (s, 1H), 6.28 (m, 1H), 4.41 (m, 1H), 3.97 (m, 2H), 3.56 (m, 2H), 2.91 (s, 3H), 2.83 (s, 3H), 1.8-2.35 (m, 4H), 1.46 (s, 9H)

### ((Me)₂N-Gly-NProO-NCH(CH₃)₂

### Example 77

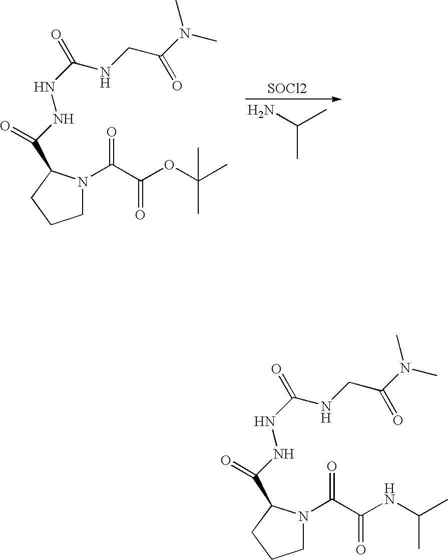

To the solution of t-BuO-Oxalyl-Pro-aza-Gly-(NMe)₂ (Example 77) (220 mg, 0.57 mmol) in DCM (5 mL), TFA (5 mL) was added slowly with stirring at 0° C., the reaction mixture was stirred overnight at rt. Solvent was evaporated and was well dried under high vacuum. The residue obtained was dissolved DCM (15 mL), to that was added with stirring SOCl₂ (0.21 mL, 2.85 mmol) followed by catalytic DMF (2 drops). The reaction mixture was further stirred overnight under argon at room temp. Solvent was evaporated on rotavap, the excess of SOCl₂ was removed under high vacuum. The residue obtained was dissolved in DCM (15 mL), to this was added slowly with stirring isopropyl amine (0.15 mL, 1.72 mmol) at 0° C. The mixture was diluted with EtOAc (30 mL) and washed with 1N HCL (10 mL), followed by 10% NaHCO₃(10 mL) and brine (10 mL). The EtOAc layer was over Na₂SO₄ and concentrated to give 220 mg of crude compound which was purified by column chromatography to give 150 mg (70%) of a white solid. 1H NMR (200 MHz, CDCl₃) δ (ppm) 9.71 (bs, 1H), 9.56 (bs, 1H), 8.11 (bs, 1H), 6.28 (bs, 1H), 4.34 (m, 1H), 4.05 (m, 2H), 3.94 (m, 1H), 3.6 (m, 2H), 2.92 (s, 3H), 2.89 (s, 3H), 1.87-2.39 (m, 4H), 1.18-1.3 (m, 6H). 13C NMR (200 MHz, CDCl₃) δ (ppm) 170.5, 164.9, 162.1, 162.0, 158.5, 62.7, 46.57, 43.1, 41.1, 36.8, 37.2, 30.1, 25.27, 23.63, 24.1.

### (S)-2-(t-Butoxyoxalyl-ethyl-amino)-butyric acid (Step 7 and

### Example 78

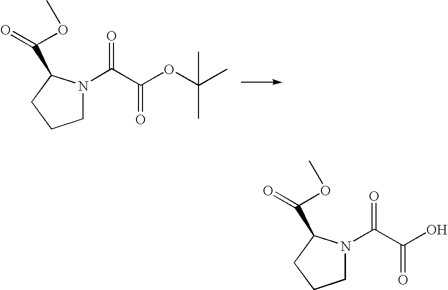

To the solution of compound (S)-2-(t-Butoxyoxalyl-ethyl-amino)-butyric acid methyl ester (prepared according to the ref Tetrahedron Letters (1998),39(23), 3957-3960.) (4.1 g, 15.94 mmol) in DCM (15 mL), TFA (15 mL) was added slowly with stirring at 0° C., the reaction mixture was stirred overnight at rt. Solvent was evaporated and was well dried under high vacuum to give 3.2 g (100%) of product as a brown viscous oil. 1H NMR (200 MHz, CDCl₃) δ (ppm) 5.09 (dd, J=3.52, 3.32 Hz, 1H), 4.53 (dd, J=3.52 Hz, 1H), 3.52-4.02 (m, 10H), 1.85-2.36 (m, 8H). 13C NMR (200 MHz, CDCl₃) δ (ppm) 170.58, 169.73, 157.76, 155.99, 155.87, 59.74, 59.22, 51.2, 48.1, 47.46, 29.85, 26.97, 23.43, 20.54, 20.28. (two rotamers visible in spectrum)

The synthesis of the compound of Example 80 has been described in: Kraus, George A. Melekhov, Alex. A direct route to acylhydroquinones from α-keto acids and α-carboxamido acids. Tetrahedron Letters (1998), 39(23), 3957-3960.

### (S)-1-Isopropylaminooxalyl-pyrrolidine-2-carboxylic acid methyl ester (Step 7 and

### Example 79

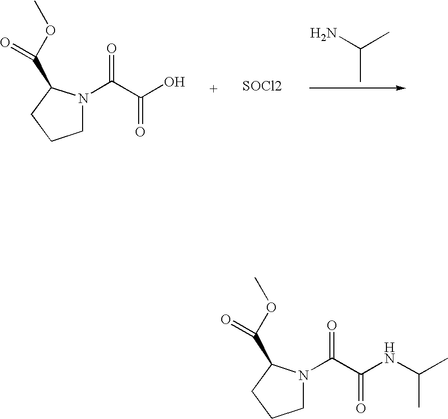

To a solution of (S)-2-(t-Butoxyoxalyl-ethyl-amino)-butyric acid (Example 78) (3.2 g, 15.91 mmol) in DCM (30 mL) was added with stirring SOCl₂ (5.81 mL, 79.53 mmol) followed by catalytic DMF (2 drops), reaction mixture was further stirred overnight under argon at rt. Solvent was evaporated on rotavap, the excess of SOCl₂ was removed under high vacuum. The residue obtained was dissolved in DCM (30 mL), to this was added slowly with stirring isopropyl amine (3.4 mL, 39.77 mmol) at 0° C. The mixture was diluted with EtOAc (50 mL) and washed with 1N HCL (10 mL), followed by 10% NaHCO₃(10 mL) and brine (10 mL). The EtOAc layer was dried (Na₂SO₄) and concentrated to give 3.4 g (88%) of desired compound as a dark brown viscous oil. R_{f}=0.32 (1:2 EtOAc/Pentane). ¹H NMR (200 MHz, CDCl₃) δ (ppm) 7.35 (bs, 2H), 5.17 (dd, J=3.92, 3.7 Hz, 1H), 4.4 (dd, J=4.3, 4.1 Hz, 1H), 3.48-4.12 (m, 10H), 1.66-2.34 (m, 7H), 1.1-1.22 (m, 10H). ¹³C NMR (200 MHz, CDCl₃) δ (ppm) 172.75, 171.88, 159.74, 159.68, 159.1 158.95, 60.75, 52.25, 49.24, 48.61, 41.50, 41.45, 31.82, 28.33, 25.49, 22.27, 22.24, 22.19, 21.71. (two rotamers visible in spectrum)

### (2-Phenylamino-ethyl)-carbamic acid 9H-fluoren-9-ylmethyl ester (Step 8

### Example 80

To a mixture of phenyl ethylenediamine (3.0 g, 22.12 mmol), DIPEA (4.65 mL, 26.54

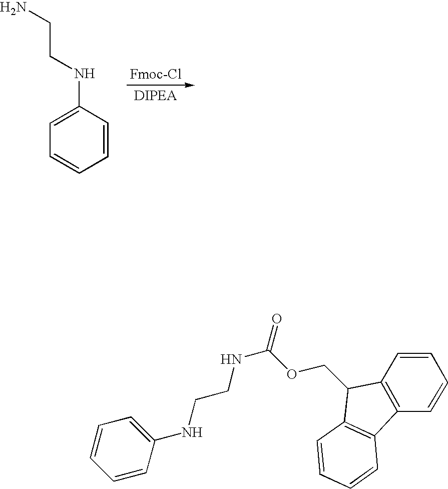

mmol) and DCM (25 mL) was added slowly with stirring and on ice-bath a solution of 9-fluorenylmethyl chloroformate (5.72 g, 22.12 mmol) in DCM (25 mL) under argon for a period of 30 min, the reaction mixture was stirred further at rt for 15 min. The reaction mixture was then washed with 1N HCL (15 mL), followed by 10% NaHCO₃(15 mL) and brine (15 mL), dried (Na₂SO₄) and concentrated to give 7.8 g of crude compound which was crystallized from EtOAc to give 6.5 g (82%) of pure product as white solid; mp. 174-175° C. R_{f}=0.54 (1:2 EtOAc/Pentane). ¹H NMR (200 MHz, DMSO-d₆) δ (ppm) 7.87 (d, J=7.24 Hz, 2H), 7.67 (m, 3H), 7.27-7.43 (m, 9H), 4.2-4.31 (m, 3H), 3.31 (bs, 4H). ¹³C NMR (200 MHz, DMSO-d₆) δ (ppm) 156.07, 143.74, 140.66, 138.6, 138.18, 129.63, 127.58, 127.03, 125.16, 121.09, 120.06, 65.56, 48.46, 46.6, 36.97.

### Boc-aza-(2-Phenylamino-ethyl)-carbamic acid 9H-fluoren-9-ylmethyl ester

### Example 81

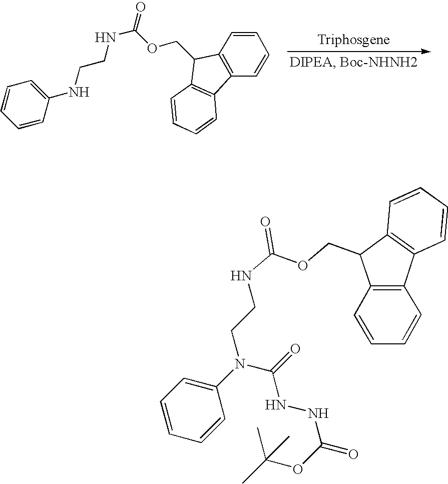

To the solution of triphosgene (578 mg, 1.95 mmol) in CH₂Cl₂ (10 mL), a mixture of (2-Phenylamino-ethyl)-carbamic acid 9H-fluoren-9-ylmethyl ester ((Example 80) (1.79 g, 4.99 mmol) and DIEA (1.0 mL, 5.99 mmol) in CH₂Cl₂ (10 mL) was added slowly over a period of 30 min. After a further 15 min of stirring, a solution of Boc-Hydrazine (990 mg, 7.49 mmol) in DCM (10 mL) was added in one portion. The reaction mixture was further refluxed for 2 hrs, evaporated to dryness, diluted with EtOAc, washed with 10% aq NaHCO₃ and brine, dried over Na₂SO₄ and evaporated to give 1.9 g of crude compound which was purified by column chromatography to give 1.1 g (42%) of a white solid; mp. 69-70° C. R_{f}=0.51 (5% methano in CH₂Cl₂). ¹H NMR (200 MHz, CDCl₃) δ (ppm) 7.67 (d, J=7.22 Hz, 2H), 7.53 (d, J=7.24 Hz, 2H), 7.18-7.42 (m, 9H), 6.33 (bs, 1H), 5.96 (bs, 1H), 5.63 (bs, 1H), 4.23 (d, J=6.84 Hz, 2H), 4.11 (t, J=6.44 Hz, 1H), 3.77 (m, 2H), 3.33 (m, 2H), 1.37 (s, 9H). ¹³C NMR (200 MHz, CDCl₃) δ (ppm) 157.77, 156.61, 156.28, 144.02, 141.23, 140.22, 130.41, 128.52, 128.27, 127.63, 127.04, 125.23, 119.91, 81.39, 66.85, 49.44, 47.18, 40.3, 28.17.

### Preparation of (S)-1-Isopropylaminooxalyl-pyrrolidine-2-carboxylic acid methyl ester

### Example 82

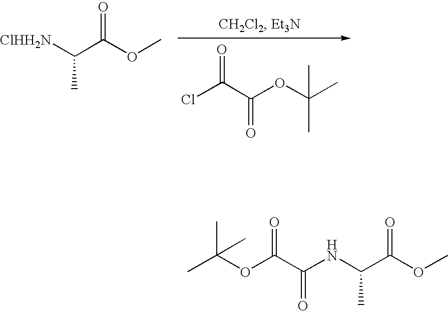

To a solution of L-alanine methylester HCl (3.02 g, 21.62 mmol) in DCM (30 mL) was added slowly with stirring triethylamine (7.15 mL, 64.87 mmol) at 0° C., followed by t-butyl oxalylchloride (3.74 g, 22.7 mmol). The reaction mixture was further stirred under argon at rt for 30 min. The mixture was diluted with EtOAc (50 mL) and washed with 1N HCL (10 mL), followed by 10% NaHCO₃ (10 mL) and brine (10 mL). The EtOAc layer was dried (Na₂SO₄) and concentrated to give 4.92 g (98%) of pure product as a dark brown viscous oil. R_{f}=0.51 (1:2 EtOAc/Pentane). ¹H NMR (200 MHz, CDCl₃) δ (ppm) 7.58 (bs, 1H), 4.55 (m, 1H), 3.74 (s, 3H), 1.53 (s, 9H), 1.44 (d, J=7.24 Hz, 3H). ¹³C NMR (200 MHz, CDCl₃) δ (ppm) 172.33, 159.02, 156.8, 84.62, 52.61, 48.42, 27.63, 17.97.

### (S)-2-(t-Butylaminooxalyl-amino)-propionic acid methyl ester

### Example 83

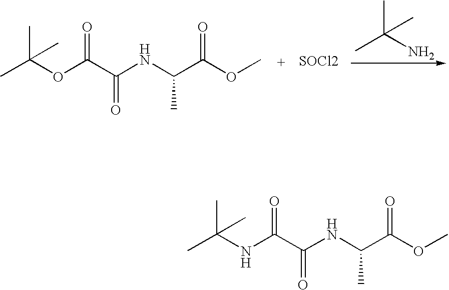

To the solution of Preparation of (S)-1-Isopropylaminooxalyl-pyrrolidine-2-carboxylic acid methyl ester (Example 82) (1.71 g, 7.4 mmol) in DCM (10 mL), TFA (10 mL) was added slowly with stirring at 0° C., the reaction mixture was stirred overnight at rt. Solvent was evaporated and was well dried under high vacuum to give 1.3 g (100%) of product as a brown viscous oil. The residue obtained was dissolved DCM (30 mL), to that was added with stirring SOCl₂ (2.7 mL, 36.97 mmol) followed by catalytic DMF (2 drops). The reaction mixture was further stirred overnight under argon at rt. Solvent was evaporated on rotavap, the excess of SOCl₂ was removed under high saccum. The residue obtained was dissolved in DCM (30 mL), to this was added slowly with stirring isopropyl amine (2.33 mL, 22.18 mmol) at 0° C. The mixture was diluted with EtOAc (50 mL) and washed with 1N HCL (10 mL), followed by 10% NaHCO₃(10 mL) and brine (10 mL). The EtOAc layer was over Na₂SO₄ and concentrated to give 1.37 g (80%) of a off-white solid; mp. 80-81° C. R_{f}=0.67 (1:2 EtOAc/Pentane). ¹H NMR (200 MHz, CDCl₃) δ (ppm) 7.94 (bs, 1H), 7.25 (bs, 1H), 4.47 (m, 1H), 3.69 (s, 3H), 1.4 (d, J=7.24 Hz, 3H), 1.33 (s, 9H). ¹³C NMR (200 MHz, CDCl₃) δ (ppm) 172.03, 160.5, 158.24, 52.53, 51.46, 48.34, 29.87, 28.2, 17.89.

### (S)-2-(t-Butylaminooxalyl-amino)-propionic acid

### Example 84

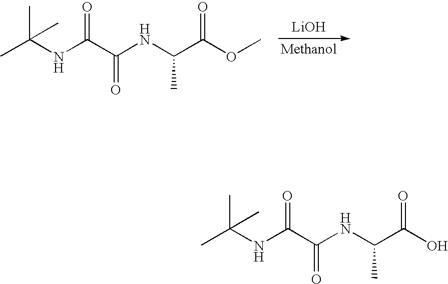

To a solution of (S)-2-(t-butylaminooxalyl-amino)-propionic acid methyl ester (Example 83) (500 mg, 2.17 mmol) in MeOH (5 mL) was added drop wise the aqueous solution of LiOH (62 mg, 2.61 mmol) in 0.5 mL of water. The reaction mixture was stirred further for 20 min at rt. MeOH was evaporated and the residue obtained was dissolved in water (10 mL), washed with ether (2×5 mL), acidified (pH=4) with 1N HCl and extracted with EtOAc (3×105 mL). The combined EtOAc layer was washed with brine (2×5 mL), dried (Na₂SO₄) and concentrated give 400 mg (85%) of a white solid; mp 118-119° C. ¹H NMR (200 MHz, CDCl₃) δ (ppm) 11.1 (bs, 1H), 8.22 (d, J=7.04 Hz, 1H), 7.43 (s, 1H), 4.5 (m, 1H), 1.45 (d, J=7.24 Hz, 3H), 1.33 (s, 9H). ¹³C NMR (200 MHz, CDCl₃) δ (ppm) 175.64, 160.17, 158.4, 51.79, 48.32, 28.17, 17.44.

### Example 85

### Peptide Mimetic

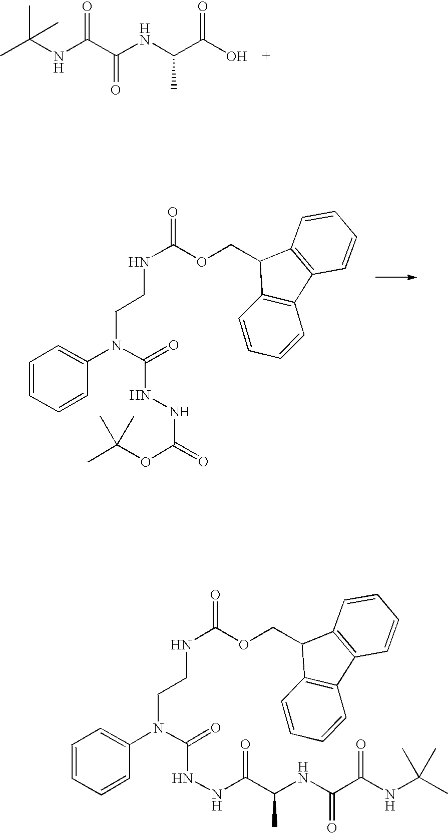

To the solution of Boc-aza-(2-Phenylamino-ethyl)-carbamic acid 9H-fluoren-9-ylmethyl ester (Example 81) (900 mg, 1.7 mmol) in DCM (10 mL), TFA (10 mL) was added slowly with stirring at 0° C., the reaction mixture was further stirred under argon at rt for 30 min (a by-product forms after stirring overnight so stirring only for 30 min is crucial). Solvent was evaporated and well dried under high vacuum, the residue obtained was dissolved in DCM (15 mL). In another flask (S)-2-(t-butylaminooxalyl-amino)-propionic acid (Example 84) (377 mg, 1.74 mmol), HOBt (247 mg, 1.83 mmol) and DCC (377 mg, 1.83 mmol) were dissolved in DCM (15 mL) and stirred for 15 min at rt, to this a mixture of above TFA salt and DIPEA (0.34 mL, 1.92 mmol) in DCM (15 mL) was added and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (15 mL), 10% NaHCO₃ (15 mL), brine (15 mL), dried (Na₂SO₄) and concentrated to give 1.1 g of crude compound which was purified by column chromatography to give 974 mg (90%) of product as a white solid; mp. 48-49° C. R_{f}=0.42 (5% MeOH in CH₂Cl₂). ¹H NMR (200 MHz, CDCl₃) δ (ppm) 9.25 (bs, 1H), 8.35 (d, J=8.6 Hz, 1H), 7.75 (d, J=7.22 Hz, 2H), 7.58 (d, J=7.24 Hz, 2H), 7.25-7.45 (m, 10H), 6.48 (bs, 1H), 5.88 (bs, 1H), 4.55-4.75 (m, 1H), 4.14-4.29 (m, 3H), 4.25 (m, 2H), 3.4 (m, 2H), 1.45 (d, J=7.04 Hz, 3H), 1.35 (s, 9H). ¹³C NMR (200 MHz, CDCl₃) δ (ppm) 170.52, 160.5, 158.29, 157.26, 156.57, 143.95, 141.22, 139.94, 130.46, 128.74, 128.1, 127.63, 127.02, 125.16, 119.92, 66.78, 51.53, 49.64, 47.74, 47.16, 39.78, 28.26, 17.67.

### Example 86

### Peptide Mimetic

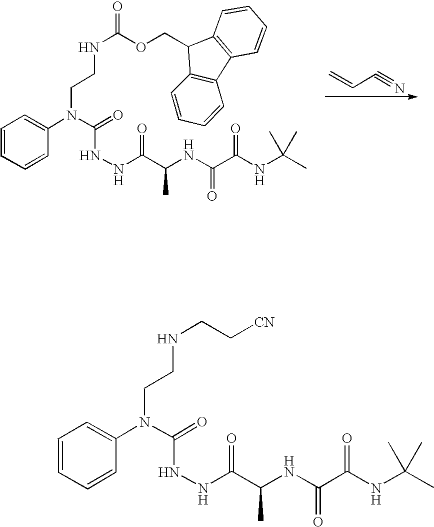

The solution of compound (Example 85) (915 mg, 1.49 mmol) in 10% piperidine in CH₂—Cl₂ (15 mL) was stirred at rt for 20 min. Solvent was evaporated and the reaction mixture was well dried under high vacuum to remove the traces of piperidine. The residue obtained was crystallized from EtOAc-pentane to give 575 mg (98%) of free amine. The free amine was dissolved in MeOH (15 mL), to that was added acrylonitrile (0.15 mL, 2.23 mmol) and the reaction mixture was stirred overnight at rt under argon. The solvent was evaporated and the crude compound was purified by column chromatography to give 333 mg (50%) of a white solid; mp. 62-63° C. R_{f}=0.42 (5% MeOH in CH₂—Cl₂). ¹H NMR (200 MHz, CDCl₃) δ (ppm) 8.14 (d, J=8.4 Hz, 1H), 7.21-7.43 (m, 6H), 4.4-4.55 (m, 1H), 3.72 (t, J=6.06 Hz, 2H), 2.84 (t, J=6.54 Hz, 2H), 2.71 (t, J=6.06 Hz, 2H), 2.41 (t, J=6.65 Hz, 2H), 1.37 (d, J=7.04 Hz, 3H), 1.3 (s, 9H). ¹³C NMR (200 MHz, CDCl₃) δ (ppm) 170.51, 160.4, 158.23, 157.09, 140.11, 130.27, 128.48, 128.23, 118.81, 51.51, 49.52, 47.69, 47.01, 44.83, 28.25, 18.61, 17.8.

### Example 87

### Peptide Mimetic

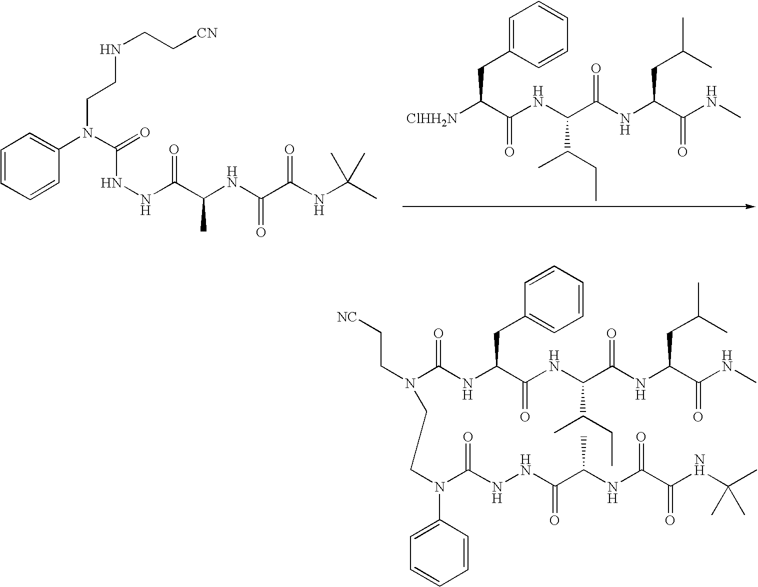

A 25 mL, one necked, round-bottomed flask, was charged with HCl salt of compound (Example 71) (363 mg, 0.82 mmol), 10 mL of CH₂Cl₂, and 10 mL of saturated aqueous NaHCO₃. The biphasic mixture was cooled to 0° C. in an ice bath. Stirring was stopped, the layers were allowed to separate, and 1.93 M solution of phosgene in toluene (0.85 mL, 1.64 mmol) was added in a single portion via syringe to the lower (organic) phase. Stirring was resumed immediately, and the ice-cooled reaction mix was stirred for 10 min at 600 rpm. The layers were separated, the aqueous phase was extracted with CH₂—Cl₂ (3×5 mL), and the combined organic layer was dried over Na₂SO₄, filtered, and concentrated. The residue obtained was dissolved in toluene (15 mL), to that was added compound (Example 86) (333 mg, 0.75 mmol). The reaction mixture was further refluxed overnight, evaporated to dryness, diluted with EtOAc, washed with 10% aq NaHCO₃ and brine, dried over Na₂SO₄ and evaporated to give crude compound which was purified by column chromatography to give (68%) of a white solid. ¹H NMR (200 MHz, CDCl₃) δ (ppm) 9.12 (bs, 1H), 8.04 (d, J=7.82 Hz, 1H), 7.56 (bs, 1H), 7.15-7.37 (m, 12H), 6.91 (bs, 1H), 6.42 (bs, 1H), 6.23 (bs, 1H), 2.28-4.52 (m, 17H), 1.29-1.69 (m, 14H), 0.6-0.77 (m, 16H).

### Example 90

### Synthesis of a Beta Turn Model Peptide

### Step 1: Boc-aza-Gly-OEt (Step 1 of Example 73)

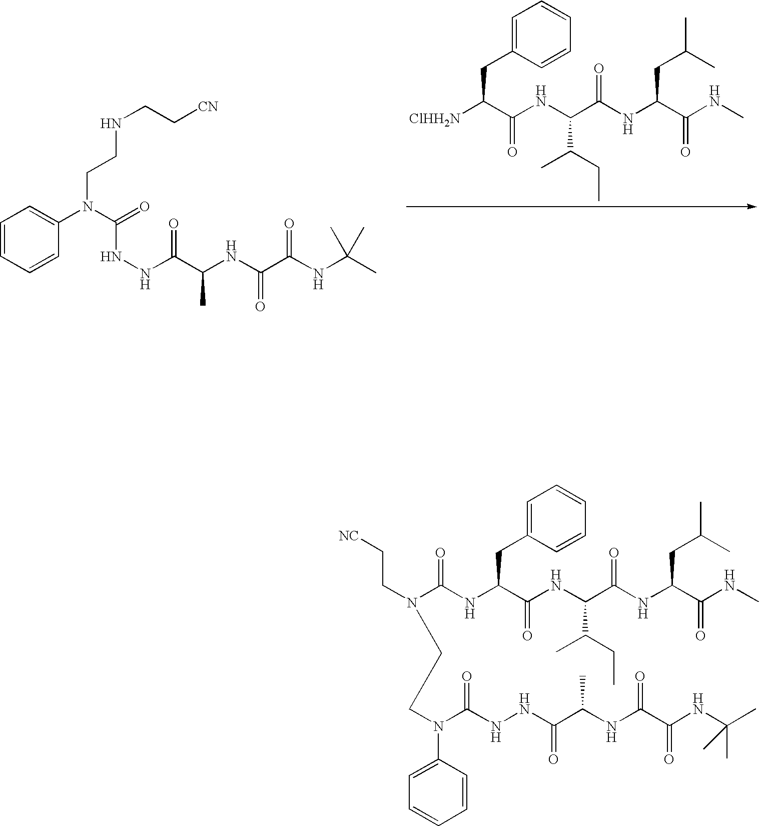

To the solution of triphosgene (3.4 g, 11.46 mmol) in CH₂Cl₂ (10 mL), a mixture of glycine ethyl ester HCl (4.0 g, 28.66 mmol) and DIEA (14.87 mL, 85.97 mmol) in CH₂Cl₂ (15 mL) was added slowly over a period of 30 min. After a further 15 min of stirring, a solution of Boc-Hydrazine (3.79 g, 28.66 mmol) in CH₂Cl₂ (15 mL) was added in one portion. The reaction mixture was further refluxed for 2 hrs, evaporated to dryness, diluted with EtOAc, washed with 10% aq NaHCO₃ and brine, dried over Na₂SO₄ and evaporated to give 5.6 g of crude compound which was purified by column chromatography to give 3.63 g (48%) of product as a sticky mass which after overnight standing at rt gave an off white solid; mp. 137-138° C. R_{f}=0.29 (5% MeOH in CH₂Cl₂). ¹H NMR (200 MHz, CDCl₃) δ (ppm) 7.2 (bs, 1H), 7.05 (s, 1H), 6.2 (t, J=5.46 Hz, 1H), 4.11 (q, J=7.04 Hz, 2H), 3.92 (d, J=5.66 Hz, 2H), 1.39 (s, 9H), 1.2 (t, J=7.04 Hz, 3H). ¹³C NMR (200 MHz, CDCl₃) δ (ppm) 171.07, 158.95, 156.43, 81.51, 61.25, 41.79, 28.09, 14.04.

### REFERENCES

1994 JOC 1937: Majer, Pavel; Randad, Ramnarayan S. A Safe and Efficient Method for Preparation of N,N′-Unsymmetrically Disubstituted Ureas Utilizing Triphosgene Journal of Organic Chemistry (1994), 59(7), 1937-8.

### Step 2:

### Boc-aza-Gly-N(Me)₂ (as in Example 75)

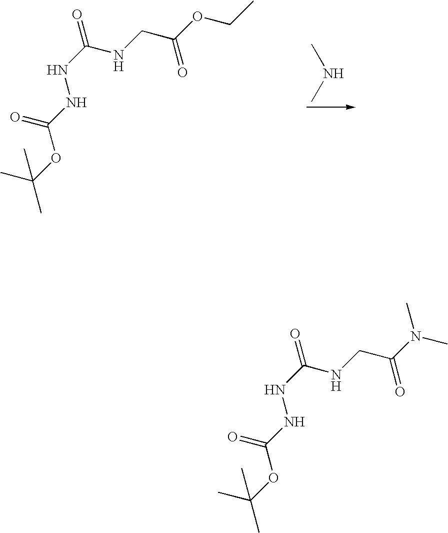

The solution of Boc-aza-Gly-OEt (Step-1 of Example 73) (3.8 g, 14.54 mmol) in 33% solution of dimethylamine in EtOH (20 mL) was stirred overnight at rt. Solvent was evaporated and was well dried under high vacuum to give 3.78 g (100%) of a off-white solid; mp. 71-72° C. R_{f}=0.24 (5% MeOH in CH₂Cl₂). ¹H NMR (200 MHz, CDCl₃) δ (ppm) 7.35 (bs, 1H), 7.07 (s, 1H), 6.48 (t, J=4.3 Hz, 1H), 4.0 (d, J=4.3 Hz, 2H), 2.92 (s, 3H), 2.89 (s, 3H), 1.38 (s, 9H). ¹³C NMR (200 MHz, CDCl₃) δ (ppm) 169.15, 158.74, 156.33, 81.08, 41.8, 36.01, 35.64, 28.13.

### Step 3:

### Boc-Pro-aza-Gly-N(Me)₂ (as in Example 76)

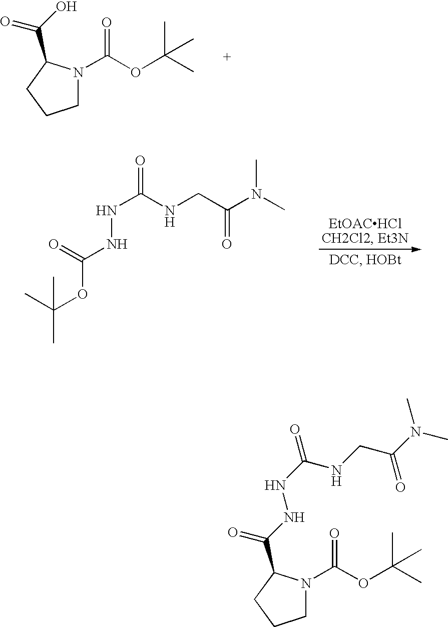

To the solution of Boc-aza-Gly-N(Me)₂ (Example 75) (1.87 g, 7.2 mmol) in EtOAc (10 mL), EtOAc saturated with HCl (10 mL) was added slowly with stirring at 0° C. The reaction mixture was stirred at rt for 30 min. Solvent was evaporated and the reaction mixture was well dried under high vacuum. In another flask Boc-Pro (1.55 g, 7.2 mmol), HOBt (1.02 g, 7.56 mmol) and DCC (1.56 g, 7.56 mmol) were dissolved in DCM (15 mL) and stirred for 15 min at rt, to this a mixture of above HCl salt and triethylamine (3.0 mL, 21.6 mmol) in DCM (15 mL) was added and the mixture was stirred overnight at rt. The precipitated DCHU was removed by filtration and the filtrate was evaporated. The additional DCHU was removed by subsequent trituration with cold EtOAc and filtration. The EtOAc solution was concentrated. The crude compound was purified by column chromatography to give 2.1 g (81%) of a white solid; mp. 74-75° C. R_{f}=0.51 (10% MeOH/CH₂Cl₂). ¹H NMR (200 MHz, CDCl₃) δ (ppm) 8.89 (bs, 1H), 7.75 (bs, 1H), 6.56 (bs, 1H), 3.87-4.22 (m, 3H), 3.41 (m, 2H), 2.91 (s, 3H), 2.88 (s, 3H), 1.68-2.1 (m, 4H), 1.36 (s, 9H). ¹³C NMR (200 MHz, CDCl₃) δ (ppm) 172.46, 169.06, 158.28, 155.33, 80.33, 58.72, 53.44, 47.07, 41.83, 36.05, 35.71, 28.33, 24.49.

### Step 3: t-BuO-Oxalyl-Pro-aza-Gly-(NMe)₂ (as in Example 77)

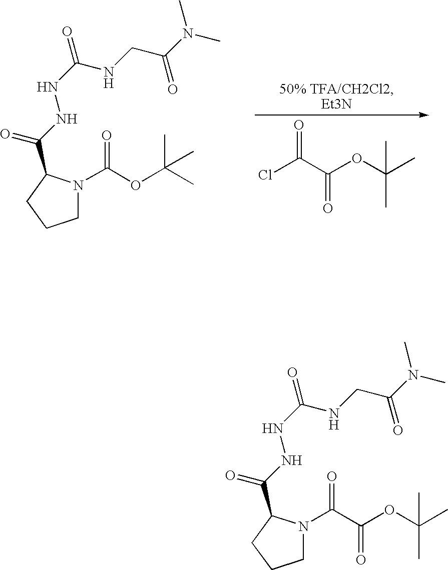

To the solution of Boc-Pro-aza-Gly-N(Me)₂ (Example 76) (0.72 g, 2.02 mmol) in DCM (10 mL), TFA (10 mL) was added slowly with stirring at 0° C., the reaction mixture was further stirred under argon at rt for 30 min. Solvent was evaporated and was well dried under high vacuum. The residue obtained was dissolved in DCM (25 mL), to that was added slowly with stirring triethylamine (0.85 mL, 6.04 mmol) at 0° C., followed by t-butyl oxalylchloride (0.33 g, 2.02 mmol) and the reaction mixture was further stirred under argon at rt for 30 min. The solvent was evaporated and the crude compound obtained was purified by column chromatography to give 250 mg (32%) of a white solid. mp. 172-173° C. R_{f}=0.36 (10% MeOH/CH₂Cl₂). ¹H NMR (200 MHz, DMSO-d₆) δ (ppm) 9.78 (bs, 1H), 8.15 (s, 1H), 6.28 (m, 1H), 4.41 (m, 1H), 3.97 (m, 2H), 3.56 (m, 2H), 2.91 (s, 3H), 2.83 (s, 3H), 1.8-2.35 (m, 4H), 1.46 (s, 9H)

### Step 4: (Me)₂N-Gly-NProO-NCH(CH3)2 (as in Example 78)

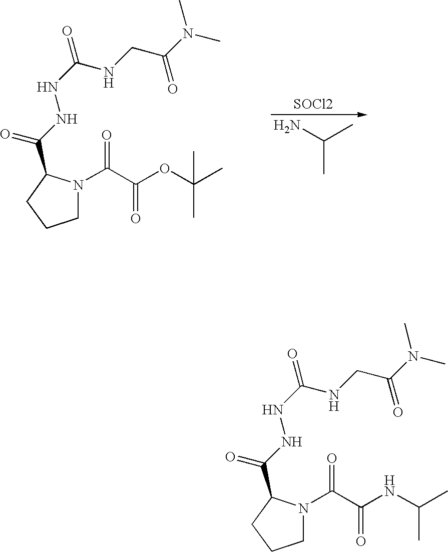

To the solution of t-BuO-Oxalyl-Pro-aza-Gly-(NMe)₂ (Example 77) (220 mg, 0.57 mmol) in DCM (5 mL), TFA (5 mL) was added slowly with stirring at 0° C., the reaction mixture was stirred overnight at rt. Solvent was evaporated and well dried under high vacuum. The residue obtained was dissolved in DCM (15 mL), to which was added with stirring SOCl₂ (0.21 mL, 2.85 mmol) followed by catalytic DMF (2 drops). The reaction mixture was further stirred overnight under argon at room temp. Solvent was evaporated on rotavap, the excess of SOCl₂ was removed under high vacuum. The residue obtained was dissolved in DCM (15 mL), to this was added slowly with stirring isopropyl amine (0.15 mL, 1.72 mmol) at 0° C. The mixture was diluted with EtOAc (30 mL) and washed with 1N HCL (10 mL), followed by 10% NaHCO₃(10 mL) and brine (10 mL). The EtOAc layer was over Na₂SO₄ and concentrated to give 220 mg of crude compound which was purified by column chromatography to give 150 mg (70) of a white solid. ¹H NMR (200 MHz, CDCl₃) δ (ppm) 9.71 (bs, 1H), 9.56 (bs, 1H), 8.11 (bs, 1H), 6.28 (bs, 1H), 4.34 (m, 1H), 4.05 (m, 2H), 3.94 (m, 1H), 3.6 (m, 2H), 2.92 (s, 3H), 2.89 (s, 3H), 1.87-2.39 (m, 4H), 1.18-1.3 (m, 6H). ¹³C NMR (200 MHz, CDCl₃) δ (ppm) 170.5, 164.9, 162.1, 162.0, 158.5, 62.7, 46.57, 43.1, 41.1, 36.8, 37.2, 30.1, 25.27, 23.63, 24.1.

### Example 91

### Synthesis of Beta Sheet Mimic

### Step 1: (2-Phenylamino-ethyl)-carbamic acid 9H-fluoren-9-ylmethyl ester (as in Example 82)

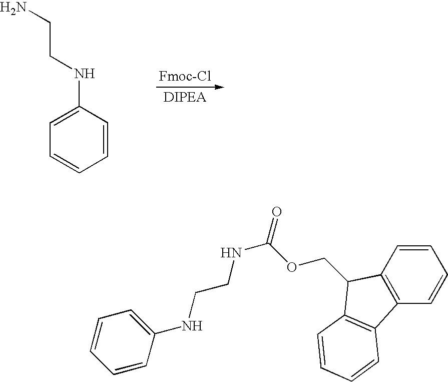

To a mixture of phenyl ethylenediamine (3.0 g, 22.12 mmol), DIPEA (4.65 mL, 26.54 mmol) and DCM (25 mL) was added slowly with stirring and ice-bath cooling a solution of 9-fluorenylmethyl chloroformate (5.72 g, 22.12 mmol) in DCM (25 mL) under argon for a period of 30 min, the reaction mixture was stirred further at rt for 15 min. The reaction mixture was then washed with 1N HCL (15 mL), followed by 10% NaHCO₃ (15 mL) and brine (15 mL), dried (Na₂SO₄) and concentrated to give 7.8 g of crude compound which was crystallized from EtOAc to give 6.5 g (82%) of a white solid; mp. 174-175° C. R_{f}=0.54 (1:2 EtOAc/Pentane). ¹H NMR (200 MHz, DMSO-d₆) δ (ppm) 7.87 (d, J=7.24 Hz, 2H), 7.67 (m, 3H), 7.27-7.43 (m, 9H), 4.2-4.31 (m, 3H), 3.31 (bs, 4H). ¹³C NMR (200 MHz, DMSO-d₆) δ (ppm) 156.07, 143.74, 140.66, 138.6, 138.18, 129.63, 127.58, 127.03, 125.16, 121.09, 120.06, 65.56, 48.46, 46.6, 36.97.

### Step 2: Boc-aza-(2-Phenylamino-ethyl)-carbamic acid 9H-fluoren-9-ylmethyl ester (as in Example 83)

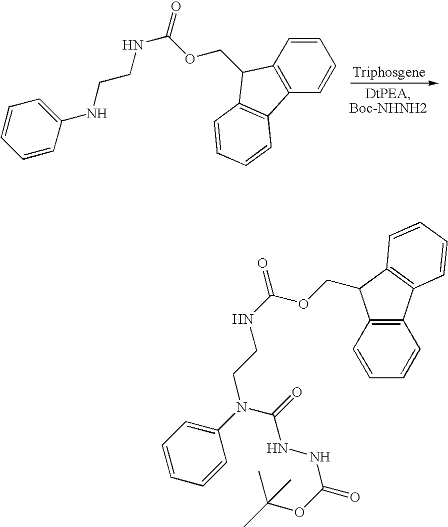

To the solution of triphosgene (578 mg, 1.95 mmol) in DCM (10 mL), a mixture of (2-Phenylamino-ethyl)-carbamic acid 9H-fluoren-9-ylmethyl ester ((Example 82) (1.79 g, 4.99 mmol) and DIEA (1.0 mL, 5.99 mmol) in CH₂Cl₂ (10 mL) was added slowly over a period of 30 min. After a further 15 min of stirring, a solution of Boc-Hydrazine (990 mg, 7.49 mmol) in CH₂Cl₂ (10 mL) was added in one portion. The reaction mixture was further refluxed for 2 hrs, evaporated to dryness, diluted with EtOAc, washed with 10% aq NaHCO₃ and brine, dried over Na₂SO₄ and evaporated to give 1.9 g of crude compound which was purified by column chromatography to give 1.1 g (42%) of product as a white solid; mp. 69-70° C. R_{f}=0.51 (5% MeOH in CH₂Cl₂). ¹H NMR (200 MHz, CDCl₃) δ (ppm) 7.67 (d, J=7.22 Hz, 2H), 7.53 (d, J=7.24 Hz, 2H), 7.18-7.42 (m, 9H), 6.33 (bs, 1H), 5.96 (bs, 1H), 5.63 (bs, 1H), 4.23 (d, J=6.84 Hz, 2H), 4.11 (t, J=6.44 Hz, 1H), 3.77 (m, 2H), 3.33 (m, 2H), 1.37 (s, 9H). ¹³C NMR (200 MHz, CDCl₃) δ (ppm) 157.77, 156.61, 156.28, 144.02, 141.23, 140.22, 130.41, 128.52, 128.27, 127.63, 127.04, 125.23, 119.91, 81.39, 66.85, 49.44, 47.18, 40.3, 28.17.

### Step 3: Preparation of (S)-1-Isopropylaminooxalyl-pyrrolidine-2-carboxylic acid methyl ester (as in Example 84)

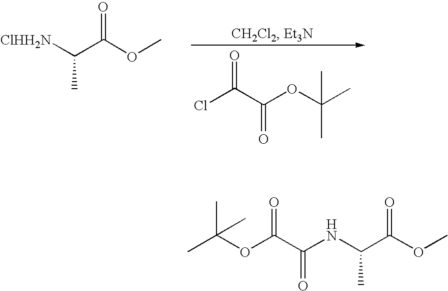

To a solution of L-alanine methylester HCl (3.02 g, 21.62 mmol) in DCM (30 mL) was added slowly with stirring triethylamine (7.15 mL, 64.87 mmol) at 0° C., followed by t-butyl oxalylchloride (3.74 g, 22.7 mmol). The reaction mixture was further stirred under argon at rt for 30 min. The mixture was diluted with EtOAc (50 mL) and washed with 1N HCL (10 mL), followed by 10% NaHCO₃(10 mL) and brine (10 mL). The EtOAc layer was dried (Na₂SO₄) and concentrated to give 4.92 g (98%) of pure product as a thick dark brown liquid. R_{f}=0.51 (1:2 EtOAc/Pentane). ¹H NMR (200 MHz, CDCl₃) δ (ppm) 7.58 (bs, 1H), 4.55 (m, 1H), 3.74 (s, 3H), 1.53 (s, 9H), 1.44 (d, J=7.24 Hz, 3H). ¹³C NMR (200 MHz, CDCl₃) δ (ppm) 172.33, 159.02, 156.8, 84.62, 52.61, 48.42, 27.63, 17.97.

### Step 4: (S)-2-(t-Butylaminooxalyl-amino)-propionic acid methyl ester (as in Example 85)

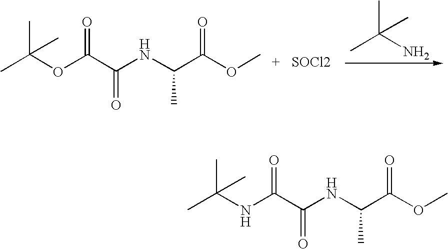

To the solution of Preparation of (S)-1-Isopropylaminooxalyl-pyrrolidine-2-carboxylic acid methyl ester (Example 84) (1.71 g, 7.4 mmol) in DCM (10 mL), TFA (10 mL) was added slowly with stirring at 0° C., the reaction mixture was stirred overnight at rt. Solvent was evaporated and well dried under high vacuum to give 1.3 g (100%) of a thick brown liquid. The residue obtained was dissolved DCM (30 mL), to that was added with stirring SOCl₂ (2.7 mL, 36.97 mmol) followed by catalytic DMF (2 drops). The reaction mixture was further stirred overnight under argon at room temp. Solvent was evaporated on rotavap, the excess of SOCl₂ was removed under high vacuum. The residue obtained was dissolved in DCM (30 mL), to this was added slowly with stirring isopropyl amine (2.33 mL, 22.18 mmol) at 0° C. The mixture was diluted with EtOAc (50 mL) and washed with 1N HCL (10 mL), followed by 10% NaHCO₃(10 mL) and brine (10 mL). The EtOAc layer was over Na₂SO₄ and concentrated to give 1.37 g (80%) of desired compound as a off-white solid; mp. 80-81° C. R_{f}=0.67 (1:2 EtOAc/Pentane). ¹H NMR (200 MHz, CDCl₃) δ (ppm) 7.94 (bs, 1H), 7.25 (bs, 1H), 4.47 (m, 1H), 3.69 (s, 3H), 1.4 (d, J=7.24 Hz, 3H), 1.33 (s, 9H). ¹³C NMR (200 MHz, CDCl₃) δ (ppm) 172.03, 160.5, 158.24, 52.53, 51.46, 48.34, 29.87, 28.2, 17.89.

### Step 5: (S)-2-(t-Butylaminooxalyl-amino)-propionic acid (as in Example 86)

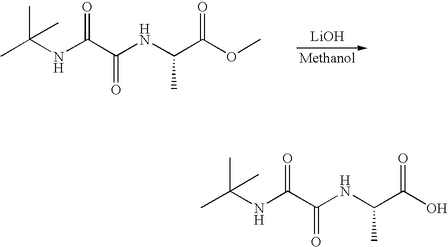

To a solution of (S)-2-(t-butylaminooxalyl-amino)-propionic acid methyl ester (Example 85) (500 mg, 2.17 mmol) in MeOH (5 mL) was added drop-wise the aqueous solution of LiOH (62 mg, 2.61 mmol) in 0.5 mL of water. The reaction mixture was stirred further for 20 min at rt. MeOH was evaporated and the residue obtained was dissolved in water (10 mL), washed with ether (2×5 mL), acidified (pH=4) with 1N HCl and extracted with EtOAc (3×105 mL). The combined EtOAc layer was washed with brine (2×5 mL), dried (Na₂SO₄) and concentrated give 400 mg (85%) of desired product as a white crystalline solid. mp 118-119° C. ¹H NMR (200 MHz, CDCl₃) (ppm) 11.1 (bs, 1H), 8.22 (d, J=7.04 Hz, 1H), 7.43 (s, 1H), 4.5 (m, 1H), 1.45 (d, J=7.24 Hz, 3H), 1.33 (s, 9H). ¹³C NMR (200 MHz, CDCl₃) δ (ppm) 175.64, 160.17, 158.4, 51.79, 48.32, 28.17, 17.44.

### Step 6: (as in Example 87)

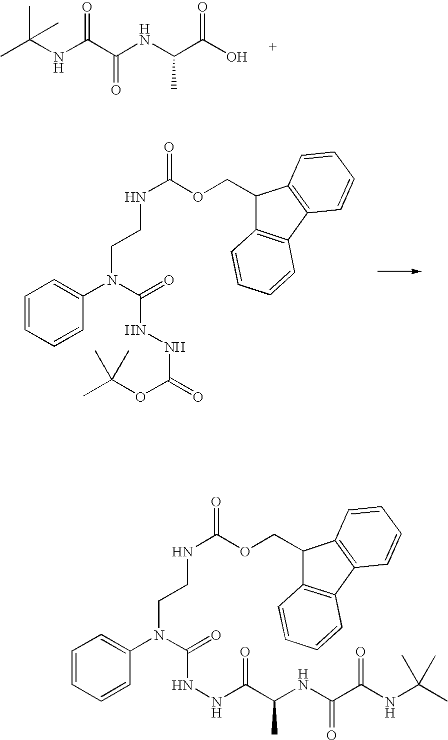

To the solution of Boc-aza-(2-phenylamino-ethyl)-carbamic acid 9H-fluoren-9-ylmethyl ester (Example 83) (900 mg, 1.7 mmol) in DCM (10 mL), TFA (10 mL) was added slowly with stirring at 0° C., the reaction mixture was further stirred under argon at rt for 30 min (After stirring overnight a by-product forms, so stirring only for 30 min is crucial). Solvent was evaporated and well dried under high vacuum, the residue obtained was dissolved in DCM (15 mL). In another flask (S)-2-(t-butylaminooxalyl-amino)-propionic acid (Example 86) (377 mg, 1.74 mmol), HOBt (247 mg, 1.83 mmol) and DCC (377 mg, 1.83 mmol) were dissolved in DCM (15 mL) and stirred for 15 min at rt, to this a mixture of above TFA salt and DIPEA (0.34 mL, 1.92 mmol) in DCM (15 mL) was added and the mixture was stirred overnight at rt. The precipitated DCHU was removed by filtration and the filtrate was evaporated. The additional DCHU was removed by subsequent trituration with cold EtOAc and filtration. The EtOAc solution was washed with 1N HCl (15 mL), 10% NaHCO₃ (15 mL), brine (15 mL), dried (Na₂SO₄) and concentrated to give 1.1 g of crude compound which was purified by column chromatography to give 974 mg (90%) of product as a white solid; mp. 48-49° C. R_{f}=0.42 (5% MeOH in CH₂Cl₂). ¹H NMR (200 MHz, CDCl₃) δ (ppm) 9.25 (bs, 1H), 8.35 (d, J=8.6 Hz, 1H), 7.75 (d, J=7.22 Hz, 2H), 7.58 (d, J=7.24 Hz, 2H), 7.25-7.45 (m, 10H), 6.48 (bs, 1H), 5.88 (bs, 1H), 4.55-4.75 (m, 1H), 4.14-4.29 (m, 3H), 4.25 (m, 2H), 3.4 (m, 2H), 1.45 (d, J=7.04 Hz, 3H), 1.35 (s, 9H). ¹³C NMR (200 MHz, CDCl₃) δ (ppm) 170.52, 160.5, 158.29, 157.26, 156.57, 143.95, 141.22, 139.94, 130.46, 128.74, 128.1, 127.63, 127.02, 125.16, 119.92, 66.78, 51.53, 49.64, 47.74, 47.16, 39.78, 28.26, 17.67.

### Step 7: (as in Example 88)

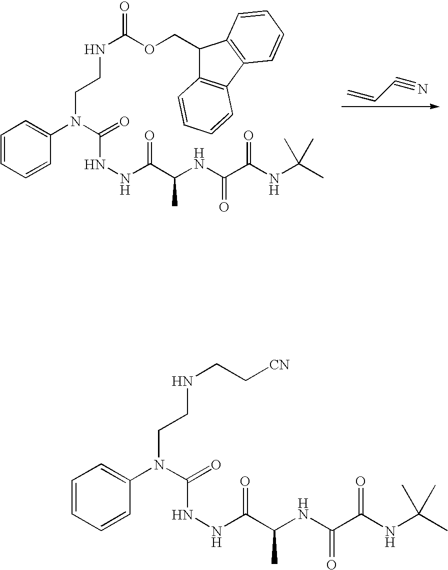

The solution of compound (Example 87) (915 mg, 1.49 mmol) in 10% piperidine in CH₂—Cl₂ (15 mL) was stirred at rt for 20 min. Solvent was evaporated and the reaction mixture was well dried under high vacuum to remove the traces of piperidine. The residue obtained was crystallized from EtOAc-pentane to give 575 mg (98%) of free amine. The free amine was dissolved in MeOH (15 mL), to that was added acrylonitrile (0.15 mL, 2.23 mmol) and the reaction mixture was stirred overnight at rt under argon. The solvent was evaporated and the crude compound was purified by column chromatography to give 333 mg (50%) of a white solid. mp. 62-63° C. R_{f}=0.42 (5% MeOH in CH₂Cl₂). ¹H NMR (200 MHz, CDCl₃) δ (ppm) 8.14 (d, J=8.4 Hz, 1H), 7.21-7.43 (m, 6H), 4.4-4.55 (m, 1H), 3.72 (t, J=6.06 Hz, 2H), 2.84 (t, J=6.54 Hz, 2H), 2.71 (t, J=6.06 Hz, 2H), 2.41 (t, J=6.65 Hz, 2H), 1.37 (d, J=7.04 Hz, 3H), 1.3 (s, 9H). ¹³C NMR (200 MHz, CDCl₃) δ (ppm) 170.51, 160.4, 158.23, 157.09, 140.11, 130.27, 128.48, 128.23, 118.81, 51.51, 49.52, 47.69, 47.01, 44.83, 28.25, 18.61, 17.8.

### Step 8: (as in Example 89)

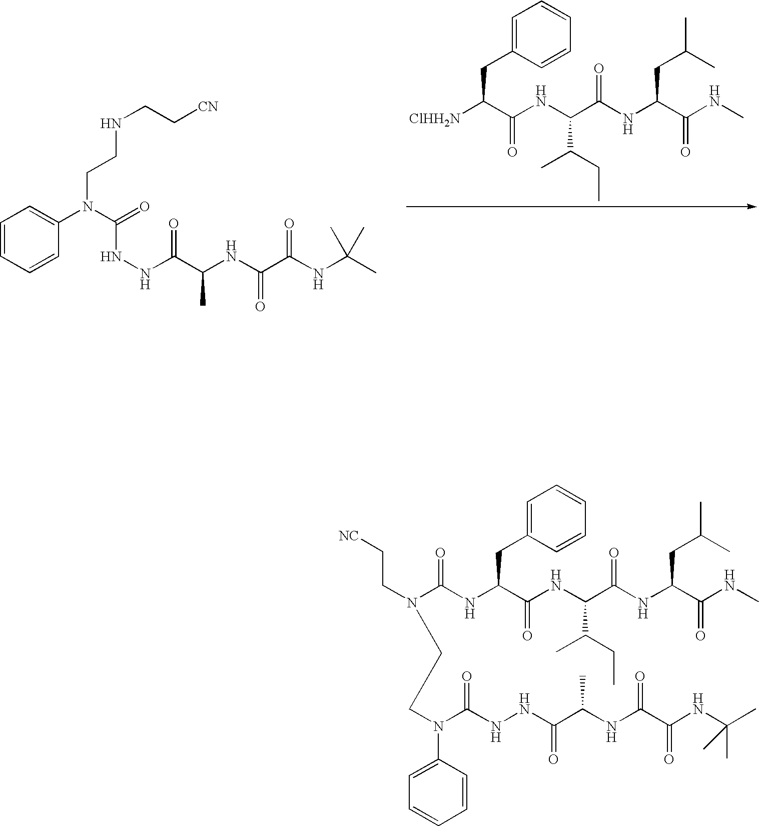

A 25 mL one-neck round-bottom flask, was charged with the HCl salt of compound (Example 71) (363 mg, 0.82 mmol), 10 mL of CH₂Cl₂, and 10 mL of saturated aqueous NaHCO₃. The biphasic mixture was cooled to 0° C. in an ice bath. Stirring was stopped, the layers were allowed to separate, and 1.93 M solution of phosgene in toluene (0.85 mL, 1.64 mmol) was added in a single portion via syringe to the lower (organic) phase. Stirring was resumed immediately, and the ice-cooled reaction mix was stirred for 10 min at 600 rpm. The layers were then separated, the aqueous phase was extracted with CH₂Cl₂ (3×5 mL), and the combined organic layer was dried over Na₂SO₄, filtered, and concentrated by rotary evaporation. The residue obtained was dissolved in toluene (15 mL), to that was added compound (Example 88) (333 mg, 0.75 mmol). The reaction mixture was further refluxed overnight, evaporated to dryness, diluted with EtOAc, washed with 10% aq NaHCO₃ and brine, dried over Na₂SO₄ and evaporated to give crude compound which was purified by column chromatography to give (68%) of a white solid. ¹H NMR (200 MHz, CDCl₃) δ (ppm) 9.12 (bs, 1H), 8.04 (d, J=7.82 Hz, 1H), 7.56 (bs, 1H), 7.15-7.37 (m, 12H), 6.91 (bs, 1H), 6.42 (bs, 1H), 6.23 (bs, 1H), 2.28-4.52 (m, 17H), 1.29-1.69 (m, 14H), 0.6-0.77 (m, 16H).

### N′-(2-Benzyloxycarbonylamino-3-phenyl-propionyl)-hydrazinecarboxylic acid t-butyl ester

### Example 92

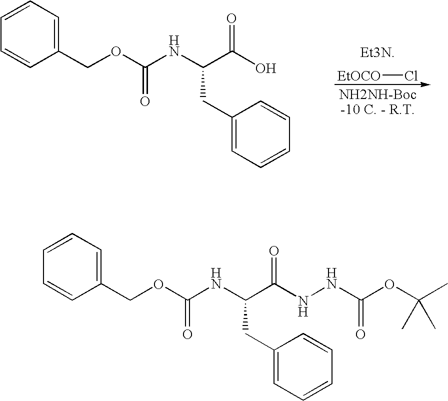

To a solution of Z-L-phenylalanine (3.6 g, 12 mmol) in dry THF (30 mL) was added slowly with stirring triethylamine (1.85 mL, 13.23 mmol) at −10° C., followed by ethylchloroformate (1.26 mL, 13.23 mmol). The reaction mixture was stirred at same temperature for 30 min under argon and the solution of t-butyl carbazate (1.59 g, 12.03 mmol) in dry THF (20 mL) was added slowly with stirring, reaction mixture was stirred further at rt for 1 h. The mixture was diluted with EtOAc (50 mL) and washed with 1N HCL (10 mL), followed by 10% NaHCO₃(10 mL) and brine (10 mL). The EtOAc layer was dried (Na₂SO₄) and concentrated to give 5.2 g crude compound which was purified by column chromatography to give 3.6 g (72%) of pure product as a colorless viscous oil which, on standing overnight at rt, crystallized as a white solid, mp 104-105° C. ¹H NMR (200 MHz, CDCl₃) δ (ppm) 8.13 (br s, 1H), 7.09-7.25 (m, 10H), 6.51 (br s, 1H), 5.43 (d, J=6.98 Hz, 1H), 4.95 (dd, J=12.34, 12.36 Hz, 2H), 4.28-4.59 (m, 1H), 3.1 (dd, J=5.9, 6.44 Hz, 1H), 2.93 (dd, J=7.92 Hz, 1H), 1.38 (s, 9H).

1.-24. (canceled)

25. A compound of formula: 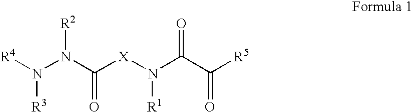
or an ester, amide, salt, stereoisomer or racemate thereof, wherein:
X is a connection between an CO-hydrazine and a NR¹-oxalic acid, oxalic ester or oxalic amide group and is either an unsubstituted 5-11-membered heteroaryl or an unsubstituted or substituted:
C₃₋₂₀-cycloalkyl group;
3-20-membered heterocyclyl group; or
linear, branched, cyclic, fused cyclic, bicyclic, or fused bicyclic C₁₋₂₀-alkyl, C₂₋₂₀-alkenyl or C₂₋₂₀-alkinyl group;
R⁵ is —SR¹⁰, —OR¹⁰ or —NR¹⁰R¹¹, provided that —NR¹⁰R¹¹ is not an amide functionality of an amino acid hydrazide, or R⁵ can cooperate with R² or R³ to form a bond or an 8 to 10 membered heterocyclic ring;
R¹⁰ and R¹¹ are independently H or a substituted or unsubstituted group further defined as a C₃₋₁₄-cycloalkyl, C₅₋₁₄-aryl, 3-14-membered heterocyclyl or heteroaryl, linear, or branched C₁₋₁₄-alkyl, C₂₋₁₄-alkenyl, or C₂₋₁₄-alkinyl group;
R³ and R⁴ together may constitute a double bond to a group R¹², wherein R¹² is a group further defined as a substituted or unsubstituted C₃₋₁₄-cycloalkyl, 3-14-membered heterocyclyl or heteroaryl, linear or branched C₁₋₁₄-alkyl, C₂₋₁₄-alkenyl, or C₂₋₁₄-alkinyl group;
R² is H or a substituted or unsubstituted group further defined as a C₃₋₁₄-cycloalkyl, 3-14-membered heterocyclyl or heteroaryl, linear or branched C₁₋₁₄-alkyl, C₂₋₁₄-alkenyl, or C₂₋₁₄-alkinyl group; and
R¹, R³, and R⁴ are independently H or a substituted or unsubstituted group further defined as a C₃₋₁₄-cycloalkyl, C₅₋₁₄-aryl, 3-14-membered heterocyclyl or heteroaryl, linear or branched C₁₋₁₄-alkyl, C₂₋₁₄-alkenyl, or C₂₋₁₄-alkinyl group;
wherein:
when X is CH₂, R³ and R⁴ are bound by single bonds, and R² is H or a group further defined as a C₃₋₂₀-cycloalkyl, C₅₋₂₀-aryl, 3-20-membered heterocyclyl or heteroaryl, linear or branched C₂₋₂₀-alkenyl, C₂₋₂₀-alkinyl, or unsubstituted C₁₋₄-alkyl group; and
X is heteroaryl;
X does not cooperate to form a ring with R³, R⁴ or R⁵.

26. The compound of claim 25, wherein X is a 3-20-membered heterocyclyl further defined as a 3-20-membered heterocyclyl other than a 1,6-naphthyridine.

27. The compound of claim 25, wherein X is an unsubstituted or substituted linear, branched, cyclic, fused cyclic, bicyclic, or fused bicyclic C₂₋₁₀-alkyl group.

28. The compound of claim 25, wherein R² is H.

29. The compound of claim 25, wherein at least two of R¹, R², R⁴ and X can cooperate to form a 3 to 10 membered ring.

30. The compound of claim 29, wherein the ring is monocyclic.

31. The compound of claim 29, wherein 1, 2, 3 or 4 rings are formed by R¹, R², R³, R⁴ and X.

32. The compound of claim 31, wherein X or R² cooperates with one of R³ or R⁴ to form the ring.

33. The compound of claim 25, wherein X does not cooperate to form a heterocycloalkyl ring with R³, R⁴ or R⁵.

34. The compound of claim 25, wherein X is a chemical group of one of Formulas 2-6: 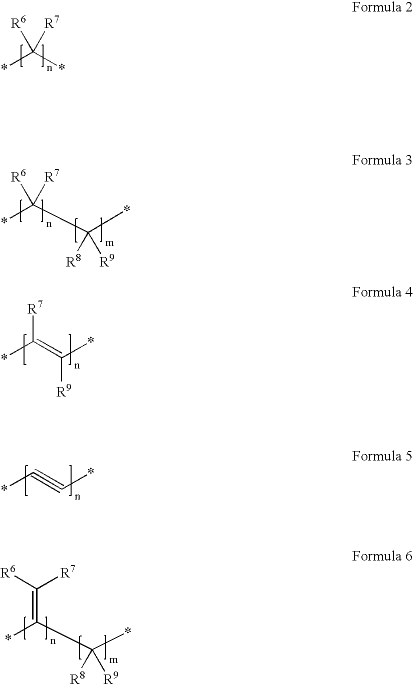
wherein:
R⁶, R⁷, R⁸ and R⁹ are independently H or a substituted or unsubstituted group further defined as a C₃₋₁₄-cycloalkyl, C₅₋₁₂ aryl, 3-12-membered heterocyclyl or heteroaryl and linear or branched C₁₋₁₄-alkyl, C₂₋₁₄-alkenyl, and/or C₂₋₁₄-alkynyl group; and
and n and m are independent integers between 0 and 5.

35. The compound of claim 34, wherein at least two of R⁶, R⁷, R⁸ and R⁹ cooperate to form a 3 to 22-membered substituted or unsubstituted or fused cycloalkyl or heterocyclic ring, or a bicycle thereof.

36. The compound of claim 34, wherein n and m are independently 1, 2, 3 or 4.

37. The compound of claim 25, wherein:
X is an NR¹-oxalic acid or ester bound group, further defined as a C₁₋₂-alkyl, guanidinylbutyl, 2-methyl-butyl, phenylethyl, p-hydroxyphenylethyl, indole-3-ylethyl, hydroxyethyl, methylthiopropyl, thioethyl, C₂₋₃-alkyl acid, C₂₋₃-alkyl acidamide, aminopentyl, 4-imidazolylethyl; or
X and R¹ cooperate to form a butyl group, forming a pyrrolidine ring with the nitrogen of the NR¹-oxalic acid or ester group.

38. The compound of claim 37, wherein X is further defined as an alpha, beta or gamma, NR¹-oxalic acid or ester bound group, wherein:
alpha means that the CO-hydrazine and the NR¹-oxalic acid, oxalic ester or oxalic amide group of Formula 1 are bound to the same atom of the X group;
beta means that the CO-hydrazine and the NR¹-oxalic acid, oxalic ester or oxalic amide group of Formula 1 are bound to neighboring atoms of the X group; and
gamma means that the CO-hydrazine and the NR¹-oxalic acid, oxalic ester or oxalic amide group of Formula 1 are bound to atoms of the X group.

39. The compound of claim 37, wherein:
the CO-hydrazine and the NR¹-oxalic acid, oxalic ester or oxalic amide group of Formula 1 are bound to the same carbon (“C-alpha”) atom of the X group;
the CO-hydrazine and the NR¹-oxalic acid, oxalic ester or oxalic amide group of Formula 1 are bound to neighboring C atoms “C-alpha” and “C-beta”, respectively, of the X group; or
the CO-hydrazine and the NR¹-oxalic acid, oxalic ester or oxalic amide group of Formula 1 are bound to C atoms “C-alpha” and “C-gamma,” respectively, separated by a C atom “C-beta” atom of the X group.

40. The compound of claim 25, wherein X is —CHR¹³—, wherein R¹³ is either H or D or the side chain of an amino acid further defined as alanine, arginine, asparagine, aspartic acid, cysteine, glutamine, glutamic acid, glycine, histidine, isoleucine, leucine, lysine, methionine, phenylalanine, proline, 4-hydroxyproline, serine, threonine, tryptophan, tyrosine, or valine.

41. The compound of claim 40, wherein X is an L-enantiomer of alanine, arginine, asparagine, aspartic acid, cysteine, glutamine, glutamic acid, glycine, histidine, isoleucine, leucine, lysine, methionine, phenylalanine, proline, 4-hydroxyproline, serine, threonine, tryptophan, tyrosine, or valine.

42. The compound of claim 25, wherein R¹⁰ and R¹¹ are independently H or a substituted or unsubstituted group further defined as a C₁₋₅-alkyl, C₂₋₁₀-alkenyl, C₂₋₁₀-alkinyl, C₃₋₁₄-cycloalkyl, C₅₋₁₄-aryl, 3-14-membered heterocyclyl, and/or 3-14-membered heteroaryl group.

43. The compound of claim 25, further defined as one of the following compounds:

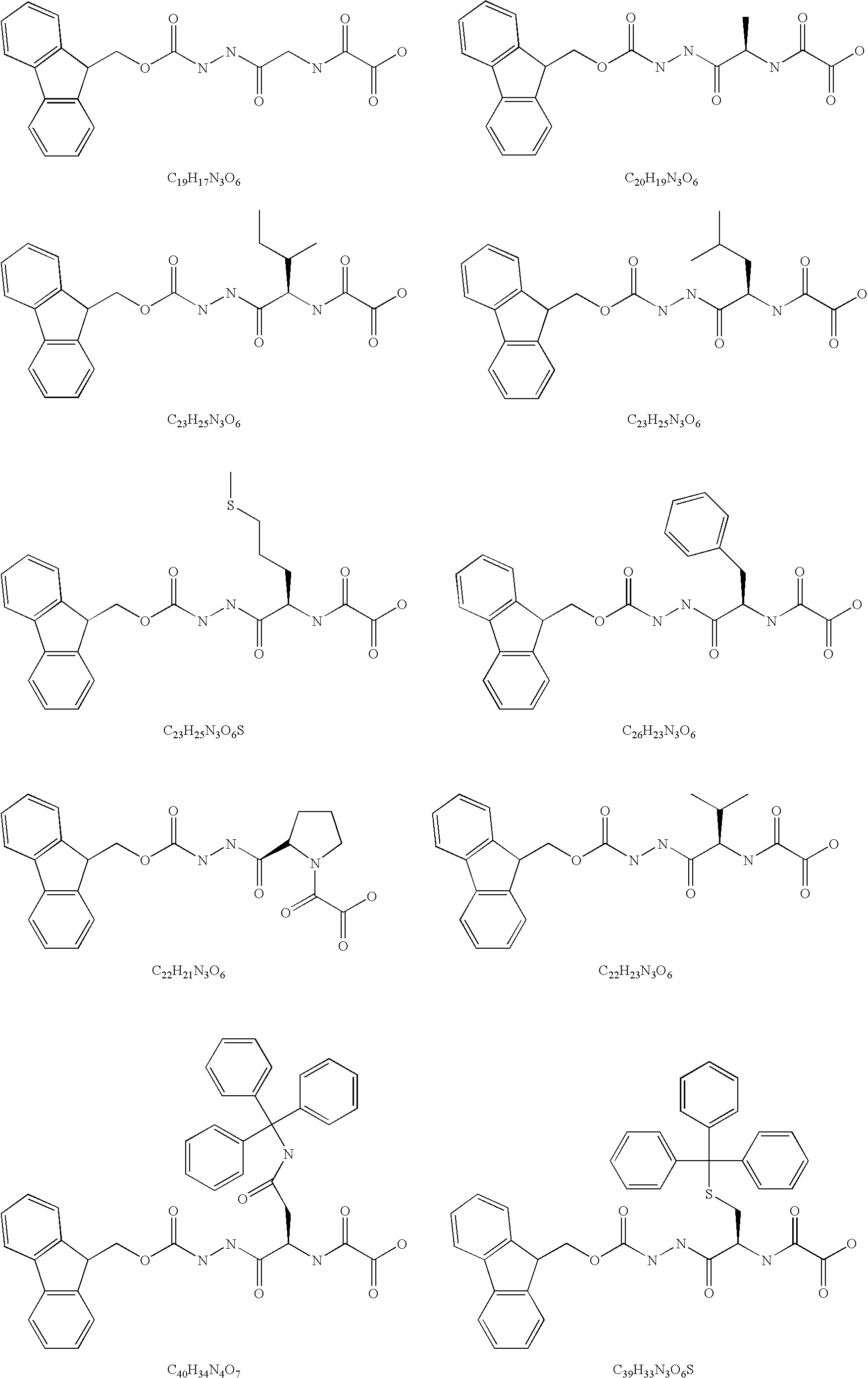

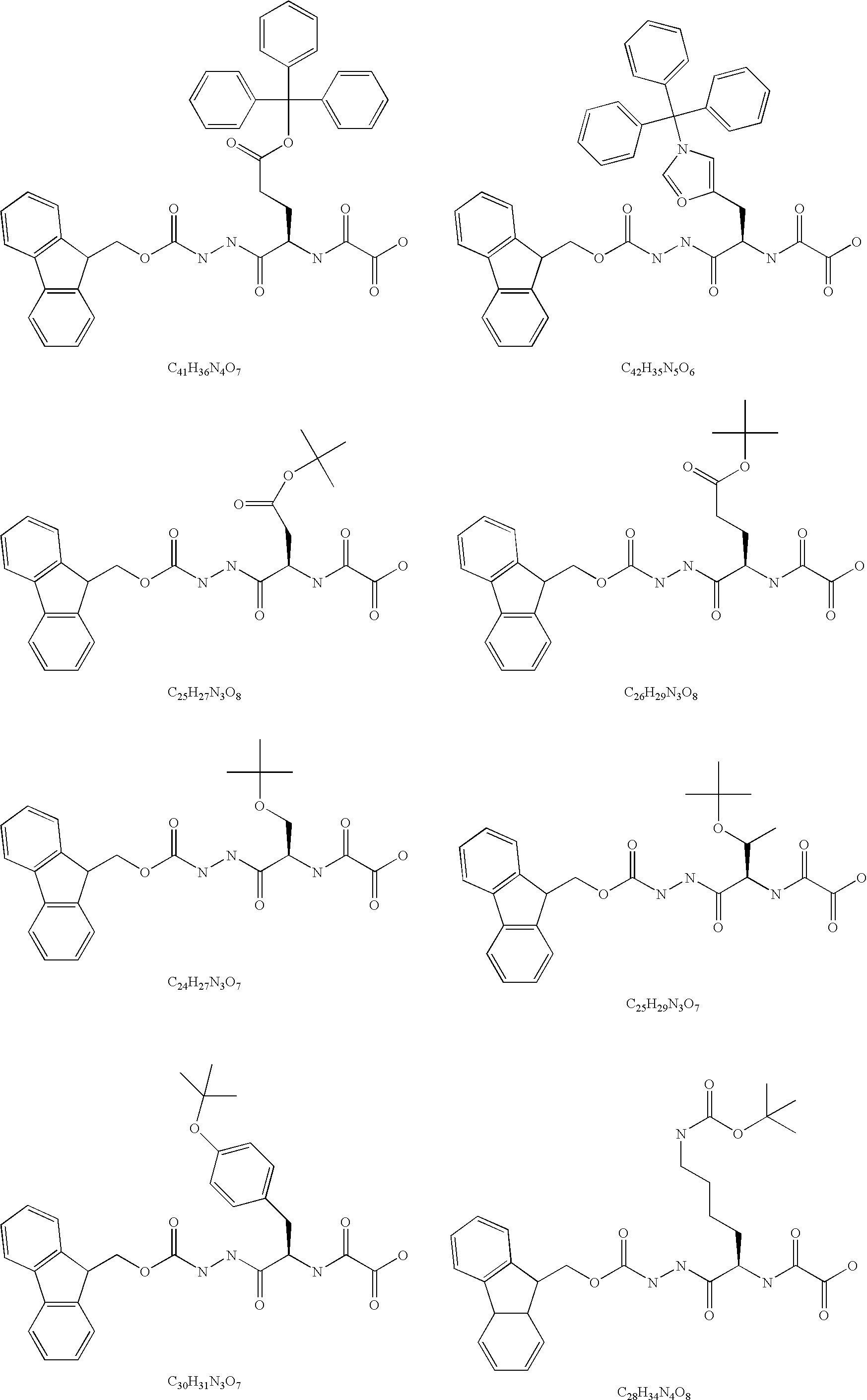

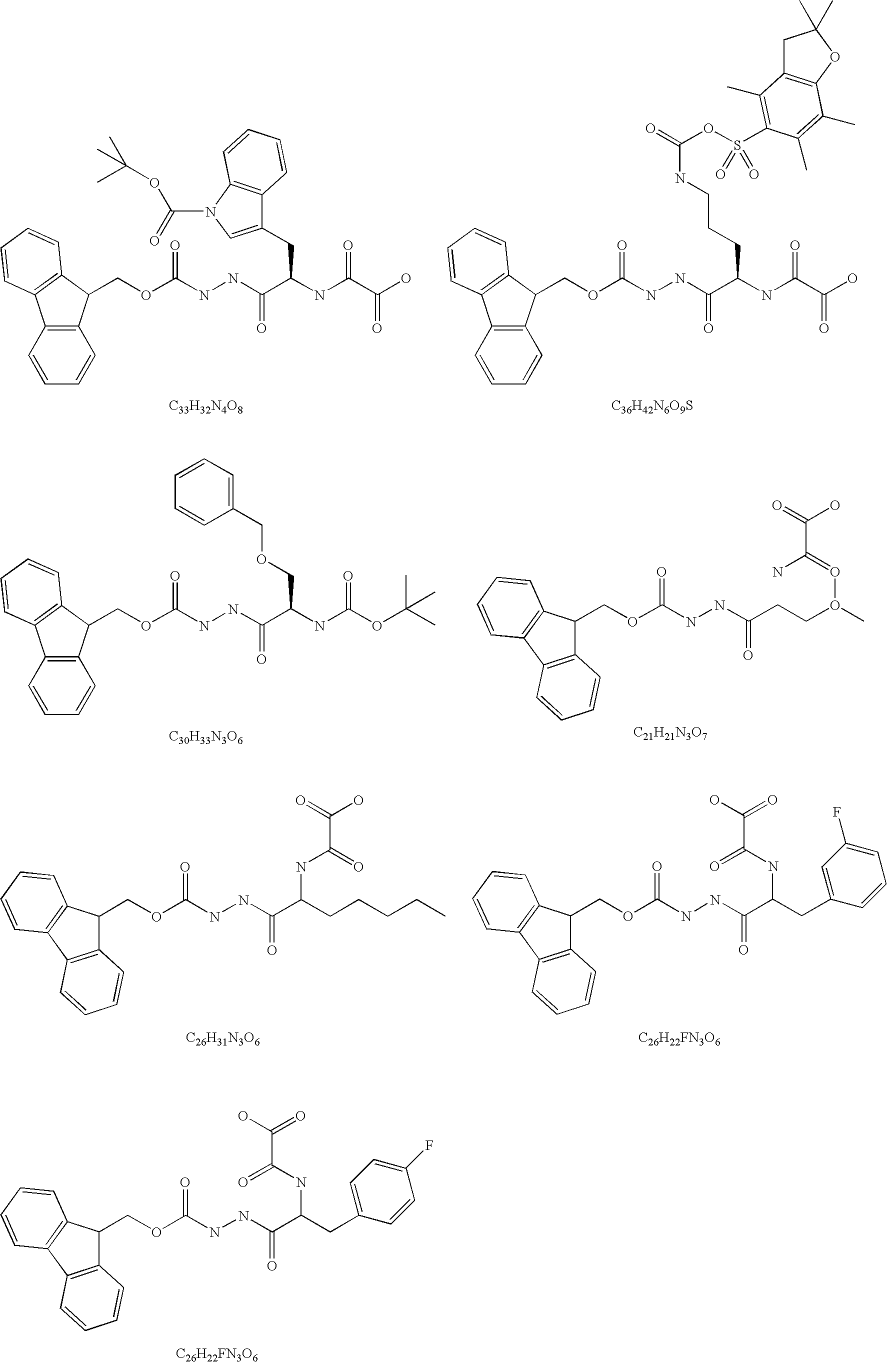

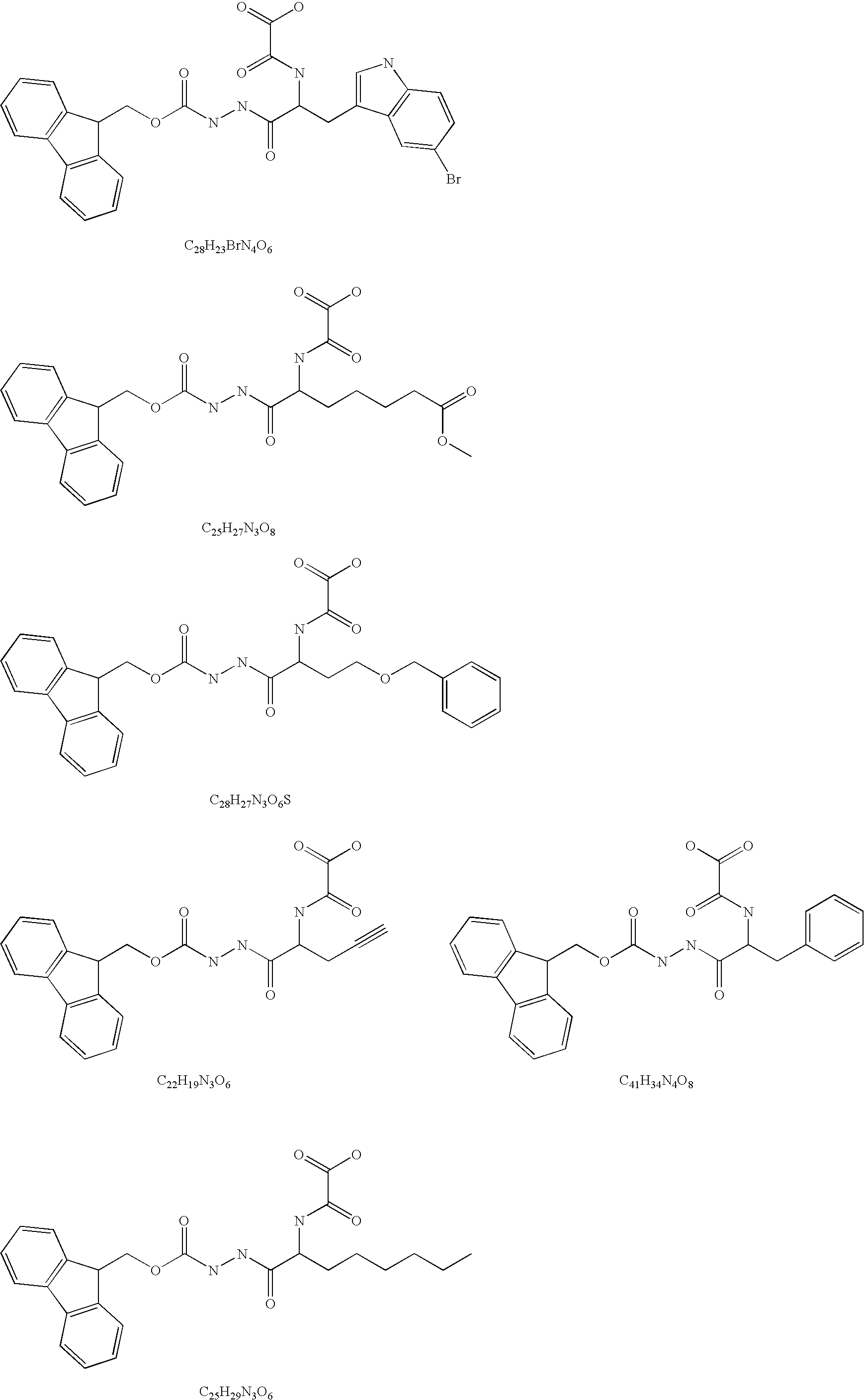

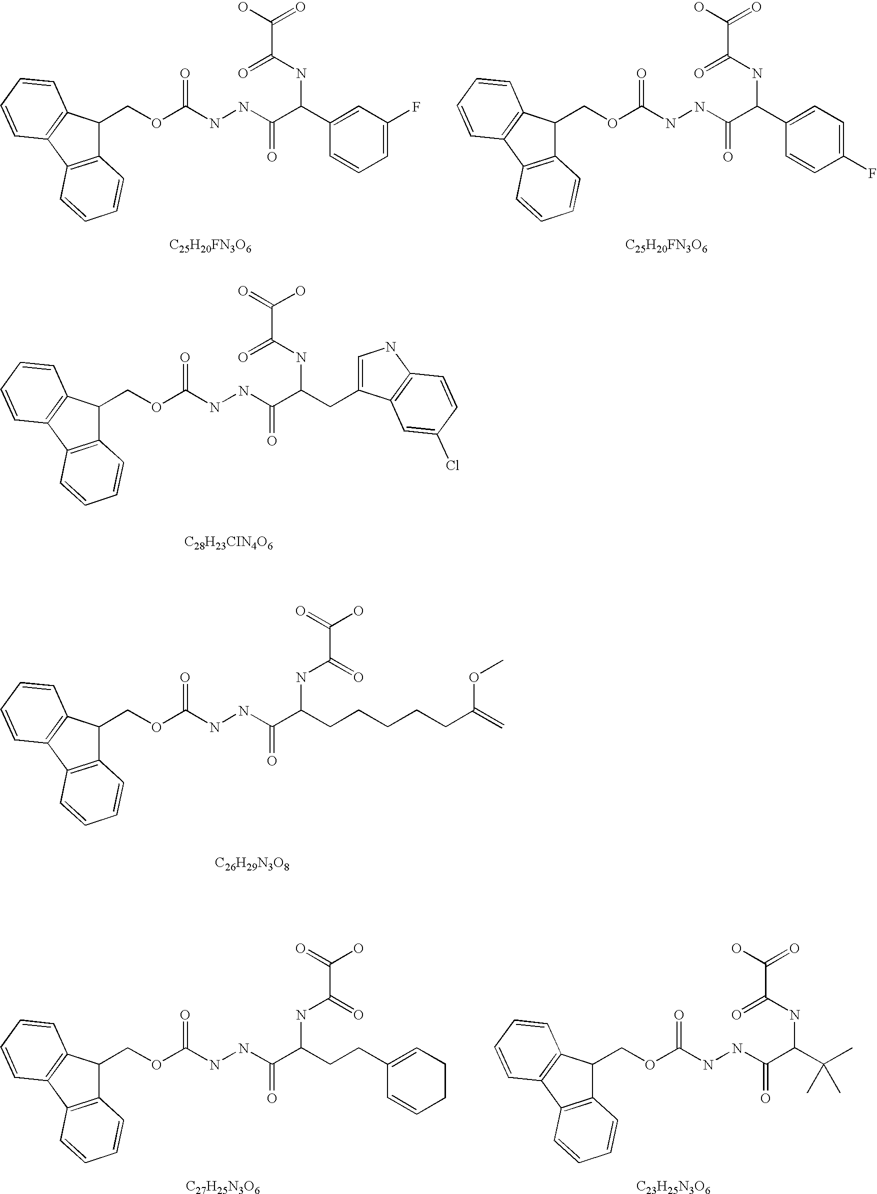

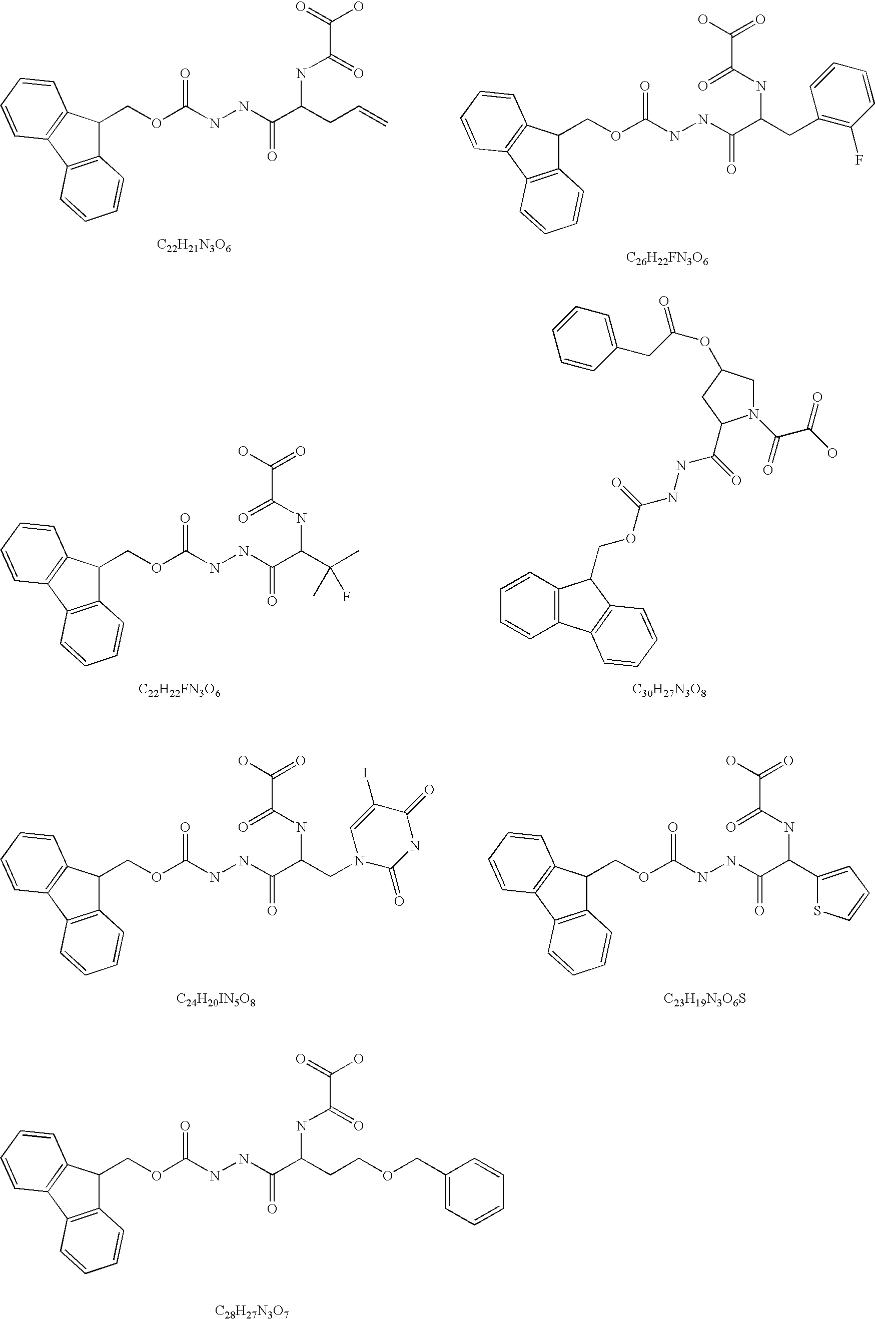

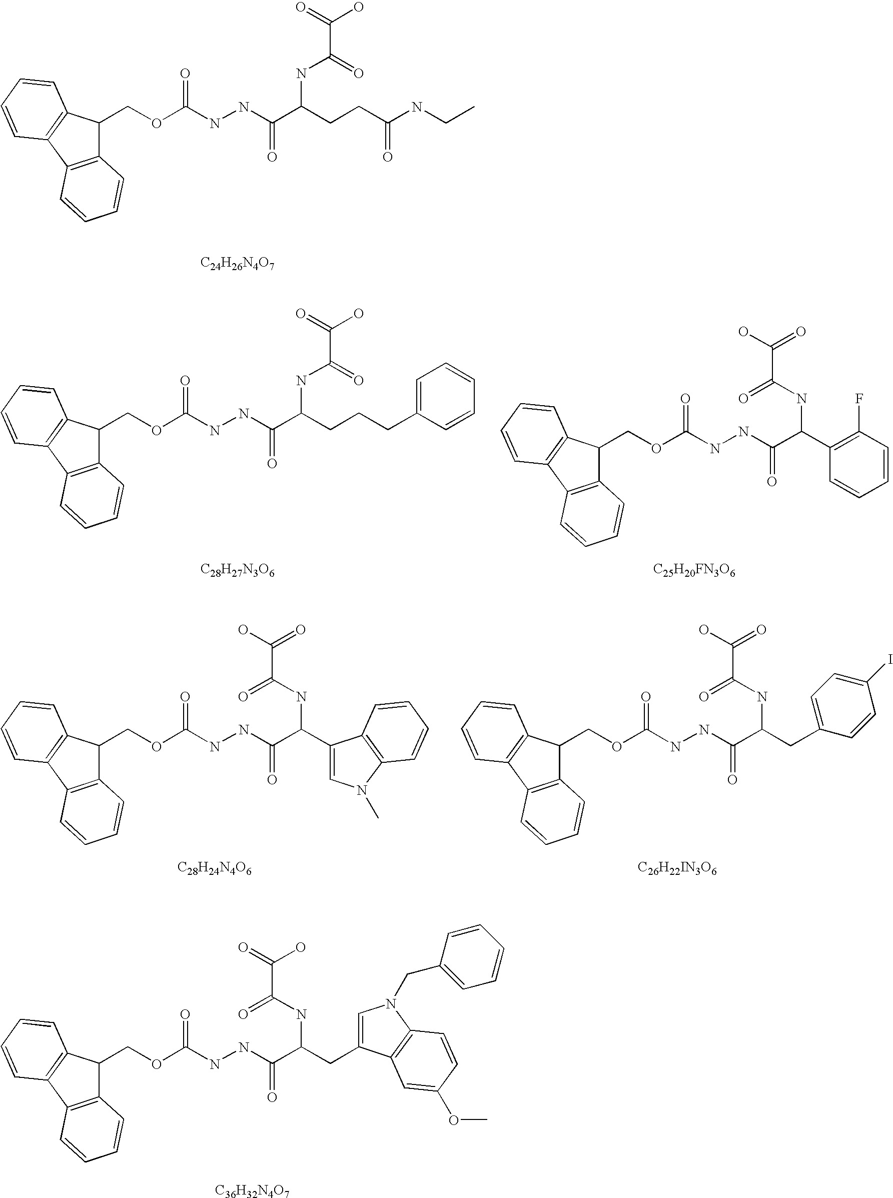

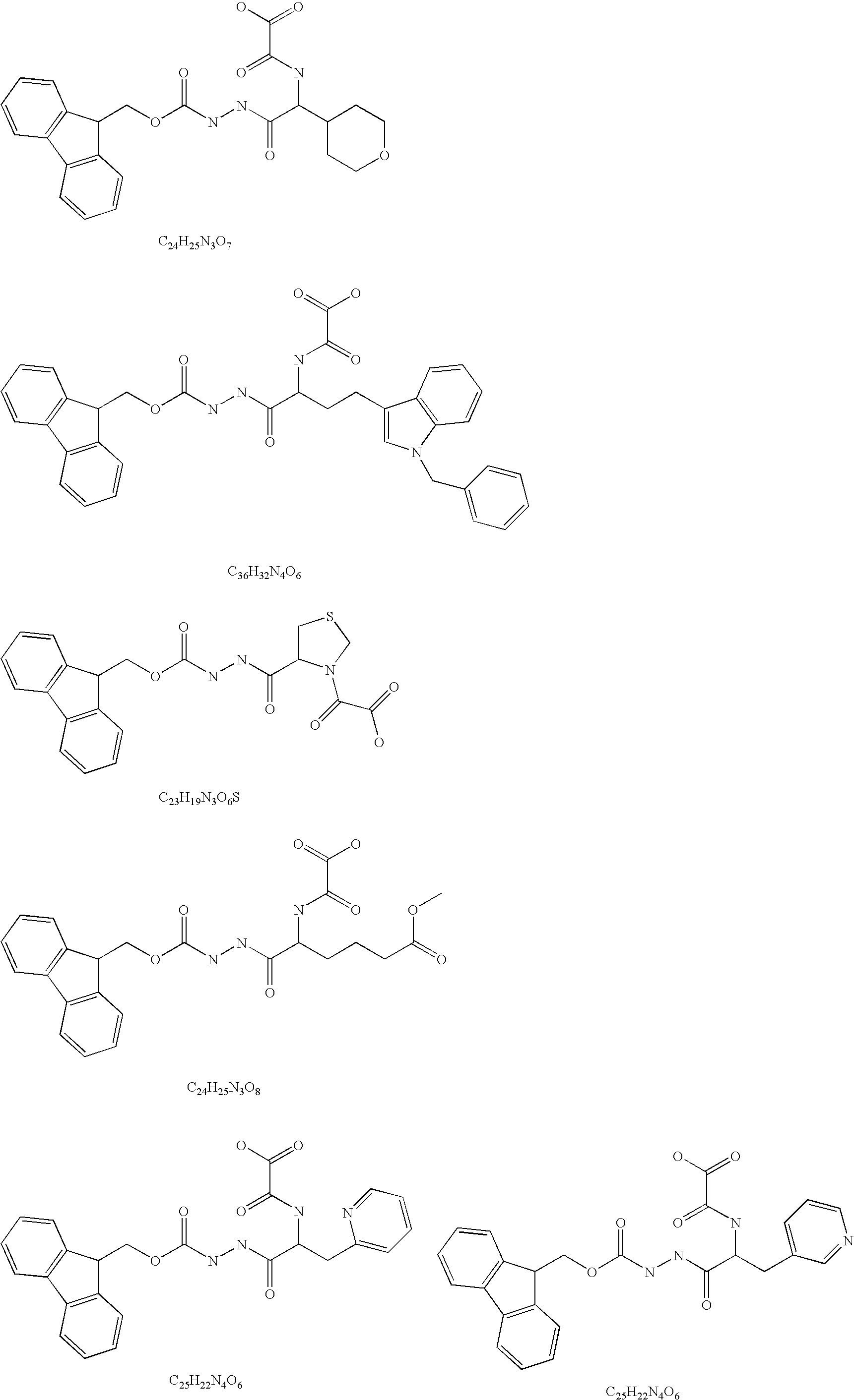

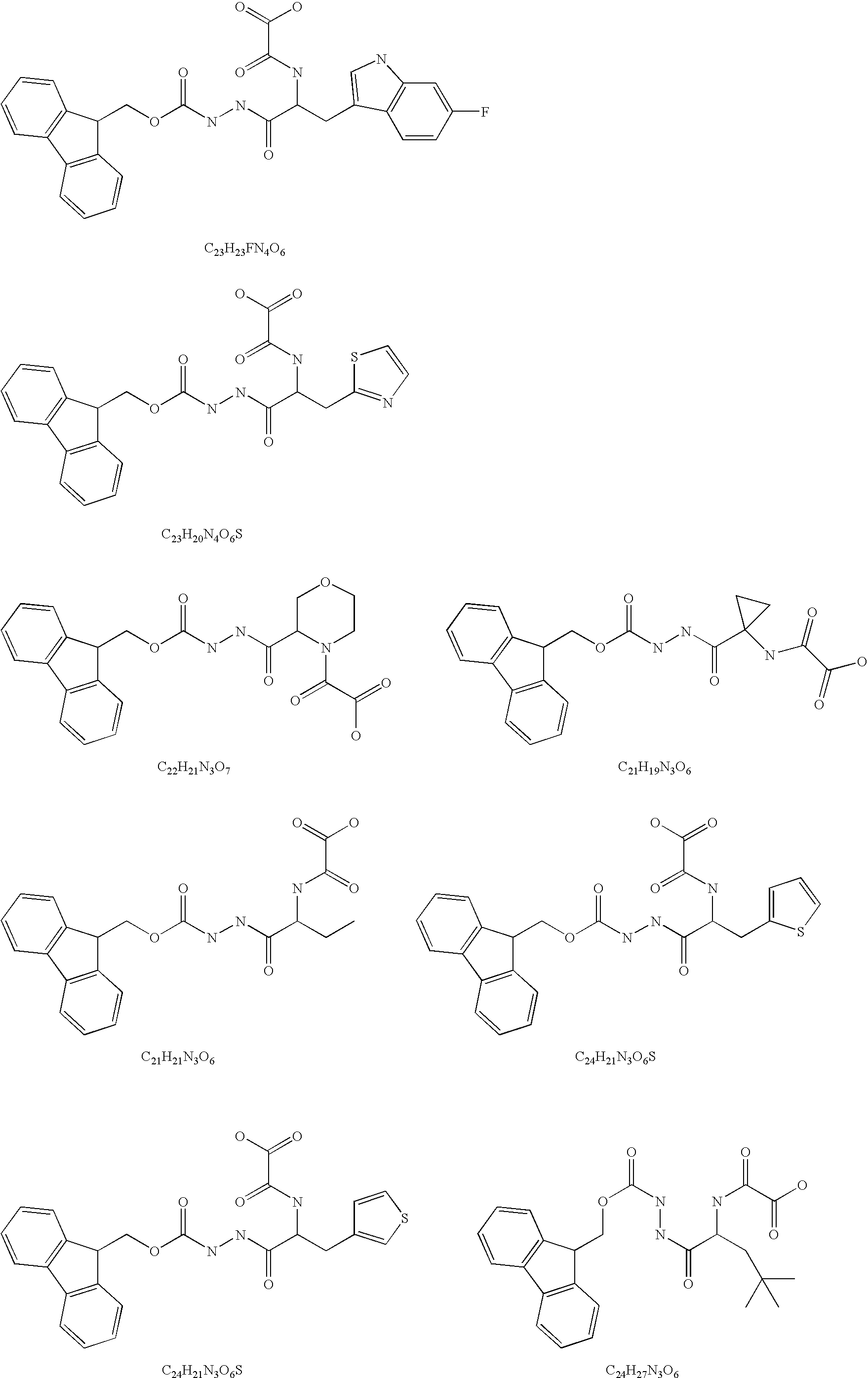

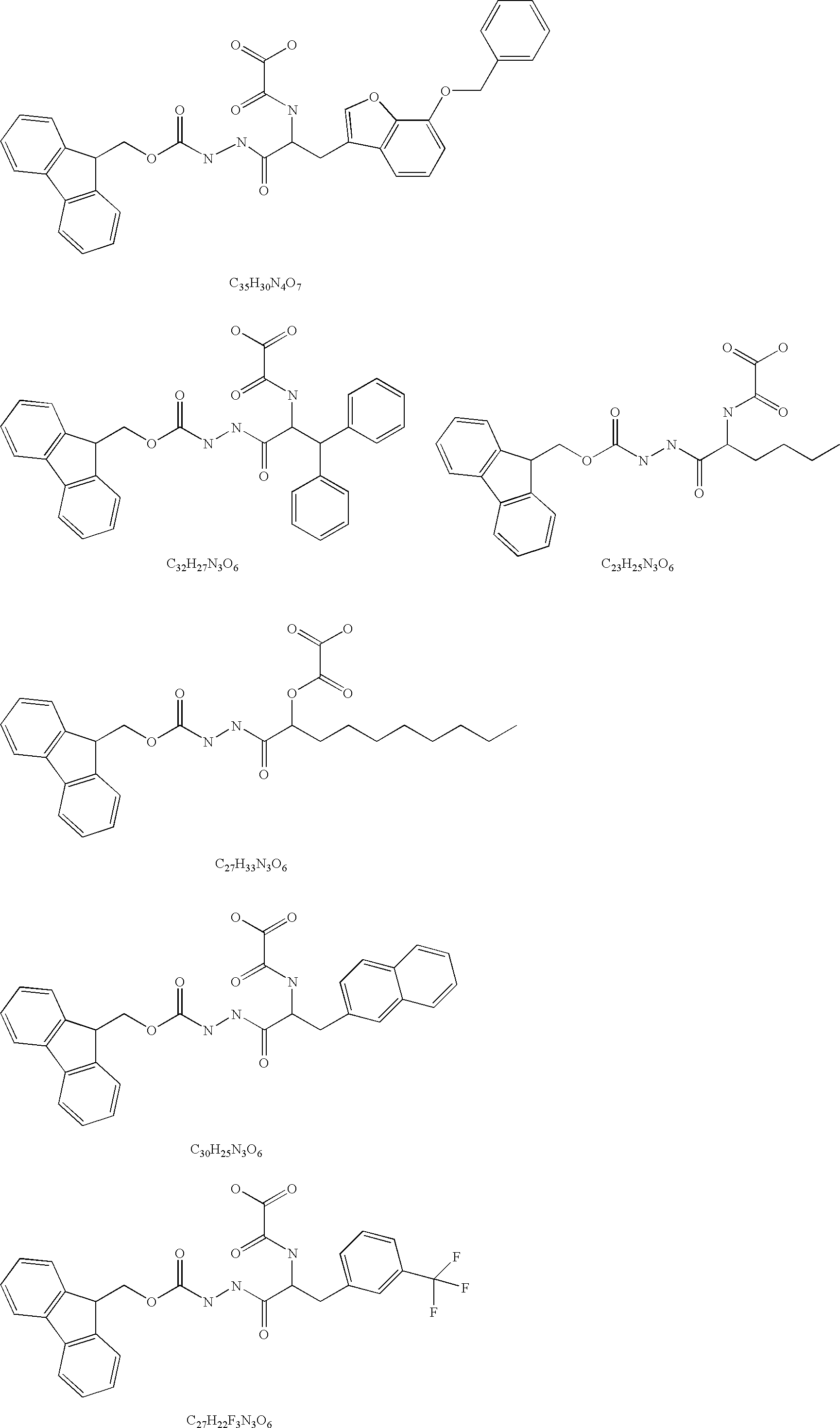

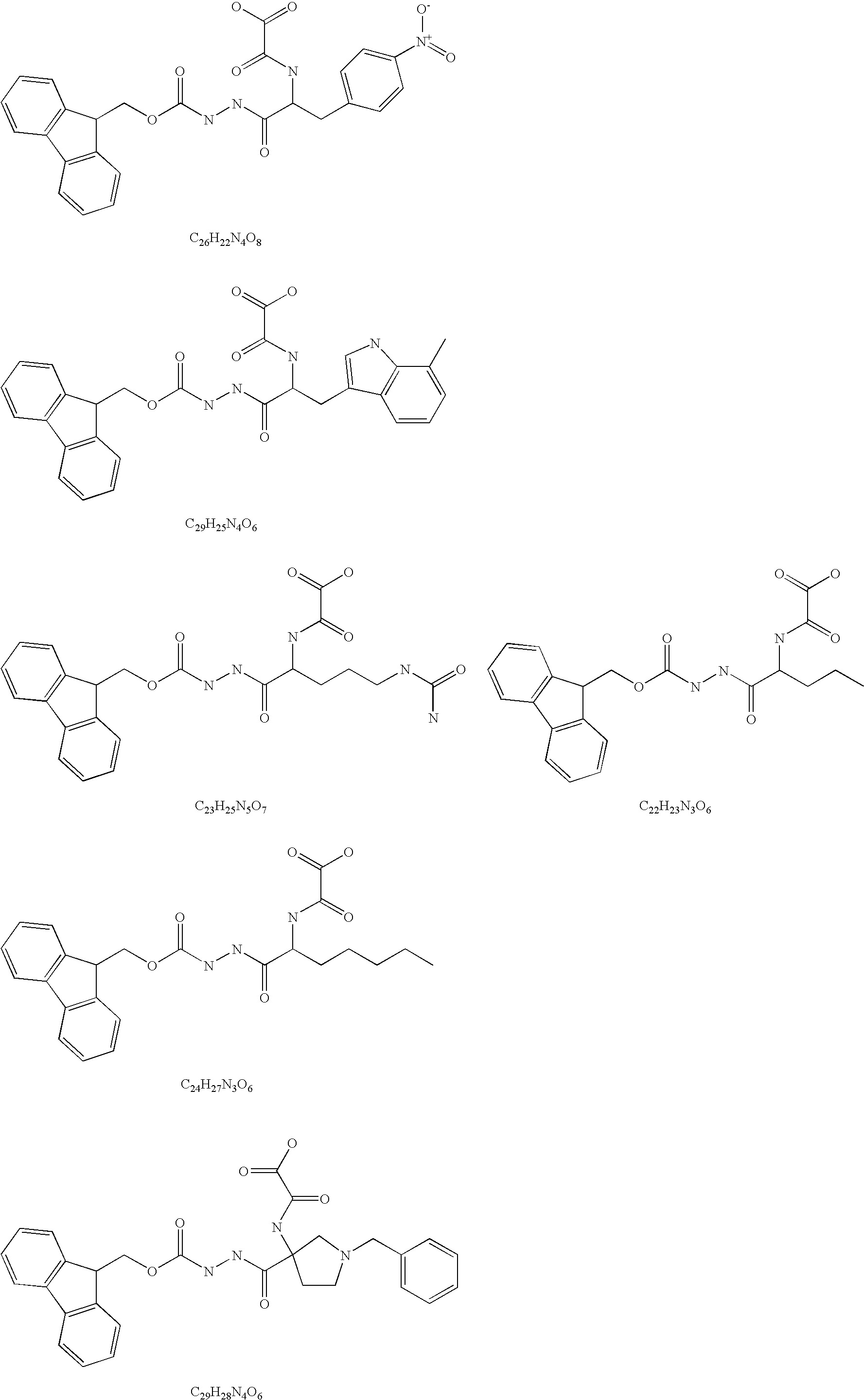

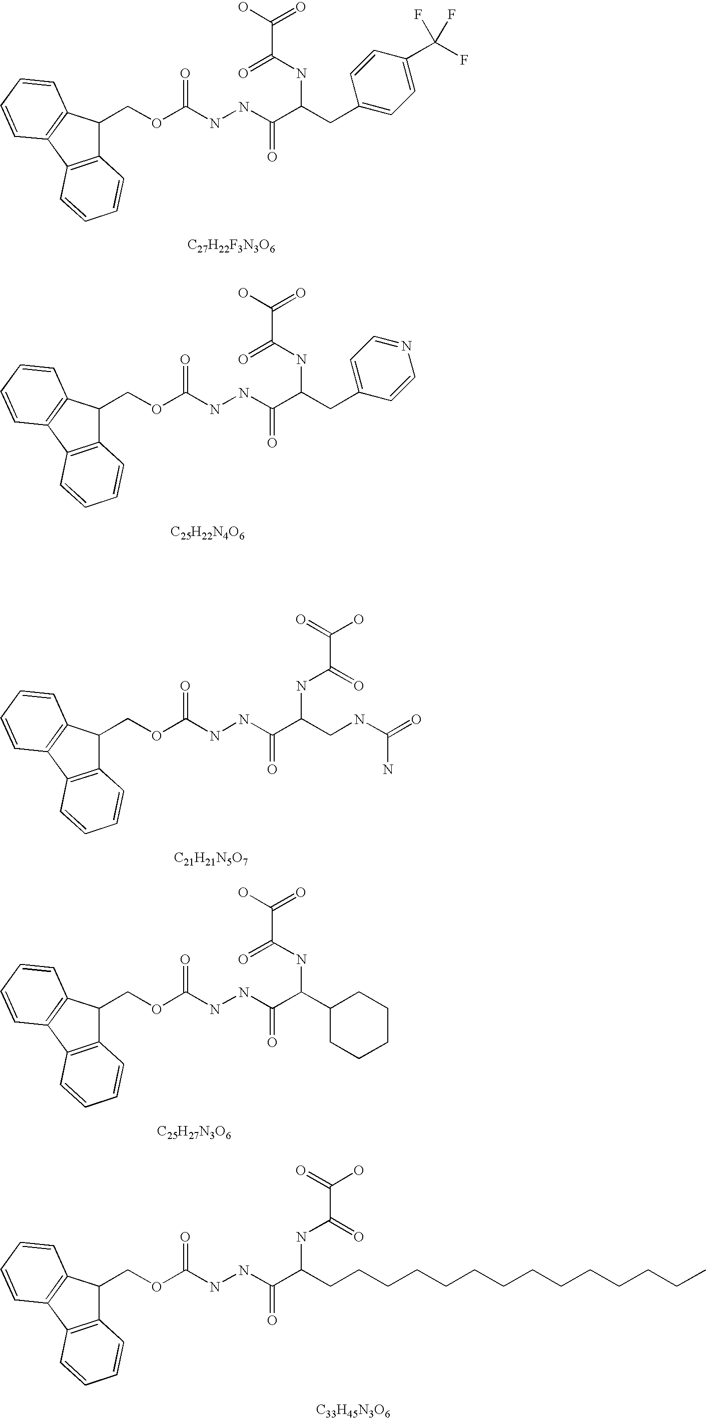

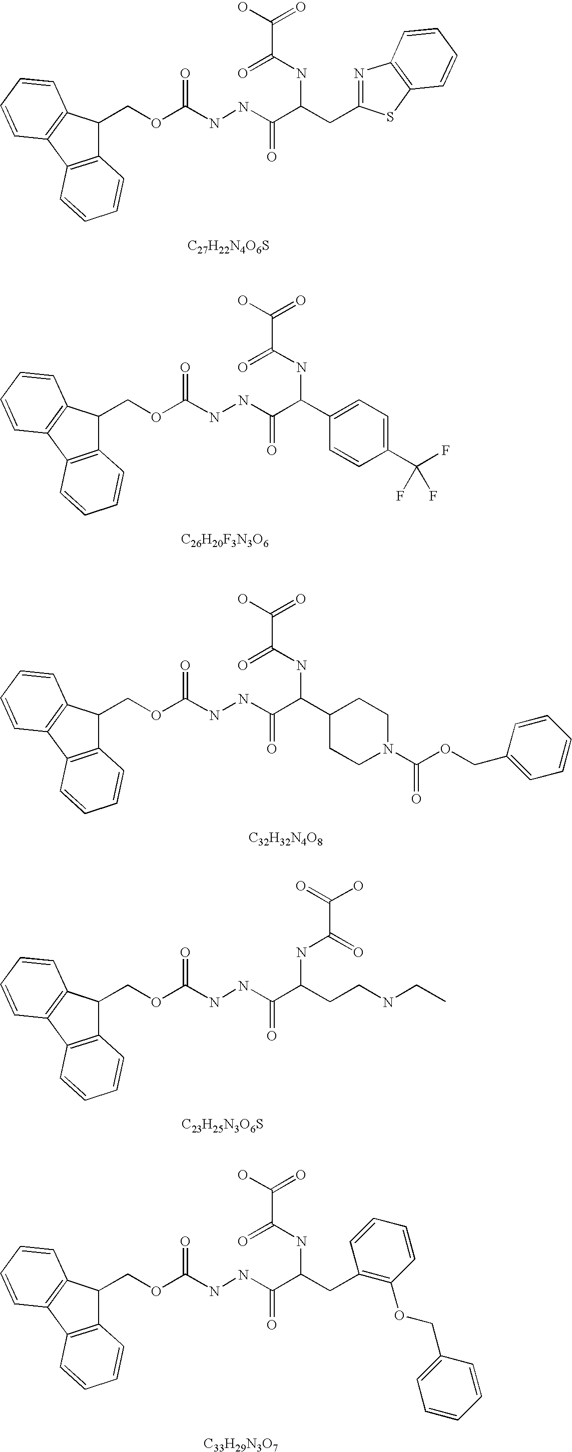

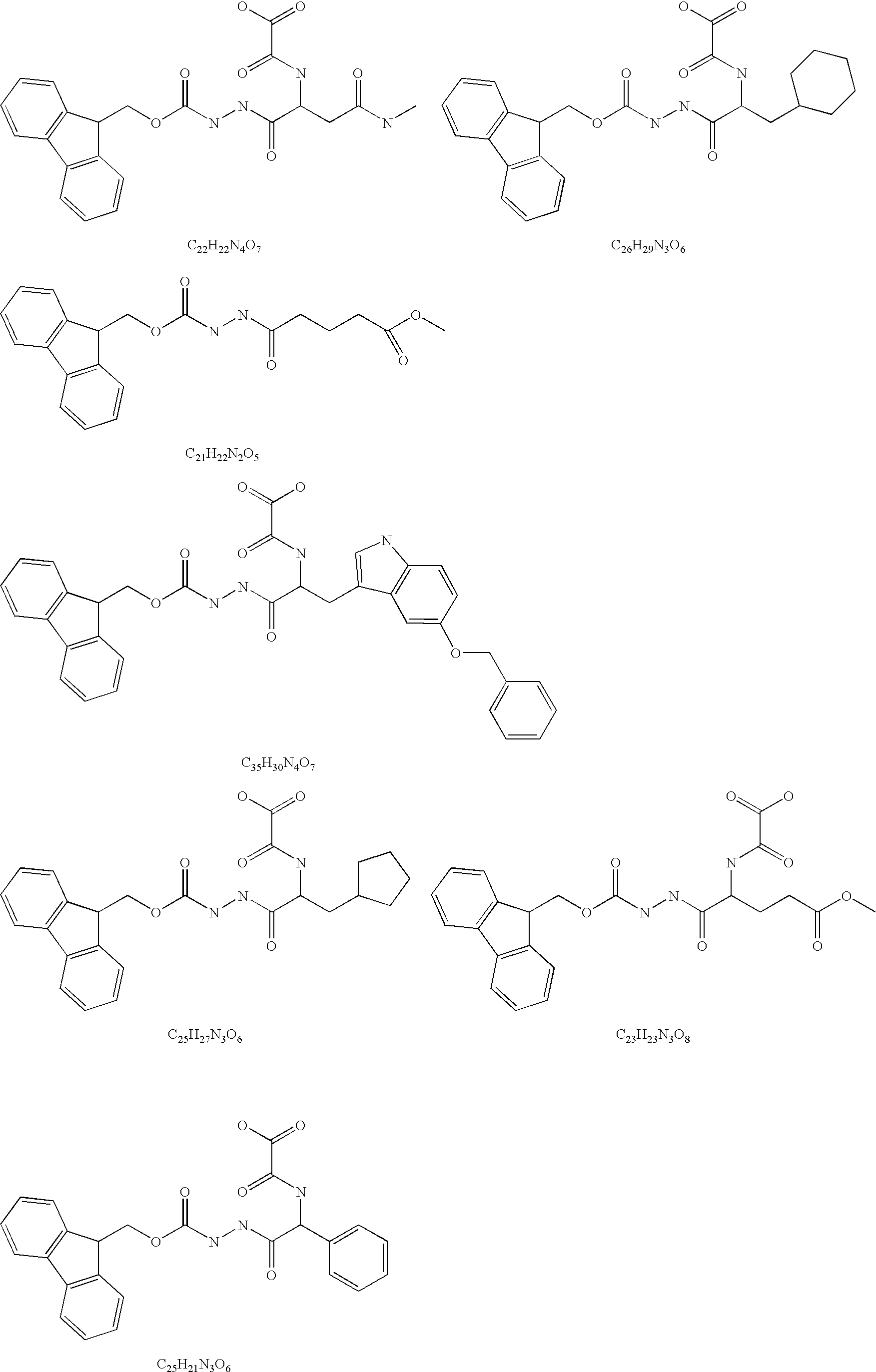

44. The compound of claim 25, wherein X is an O-, S-, N-, or P-heterosubstituted 3-20-membered heterocycle, excluding 1,6-naphthyridine.

45. The compound of claim 25, wherein R³ and R⁵ form a bond resulting in a heterocylic compound of the general Formula 10:

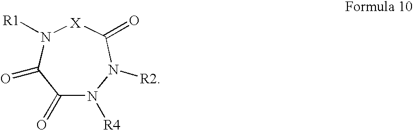

46. The compound of claim 25, wherein:
R³ or R⁴ is an N-protecting group further defined as Boc, Fmoc, Alloc, trifluoroacetyl, methoxycarbonyl, ethoxycarbonyl, benzyloxycarbonyl, 2-(trimethylsilyl)ethoxycarbonyl, 2,2,2-trichloroethoxycarbonyl, arylsulfonyl, 2-trimethylsilyl)ethylsulfonyl, or trityl; and/or
R⁵ is a carboxylic acid protecting or activating group further defined as OMe, OEt, O-t-Bu, OBn, OCHPh₂, aphenacyl ester, an alkoxyalkyl ester, a 2,2,2-trichloroethyl ester, a 2-(trimethylsilyl)ethyl ester, a 2-tosylethyl ester, or a silyl ester or activating group; and
both the protecting or activating groups can be used alternatively in their polymer- or resin bound form.

47. The compound of claim 46, wherein R⁵ is a carboxylic acid protecting or activating group further defined as an N-hydroxysuccinimid ester, 1-hydroxybenzotriazoleester, 4-nitrophenylester, or esters prepared in situ.

48. The compound of claim 47, wherein R⁵ is an ester prepared in situ using the reagents HNTU (=2-(endo-5-norbornene-2,3-dicarboximido)-1,1,3,3,-tetramethyluronium hexafluorophosphate), HOCt (=1-hydroxy-1H-1,2,3-triazole-4-carboxylate, and/or HONB (=N-hydroxy-5-norbornene-2,3-dicarboxyl.

49. The compound of claim 25, further defined as one of the following compounds:

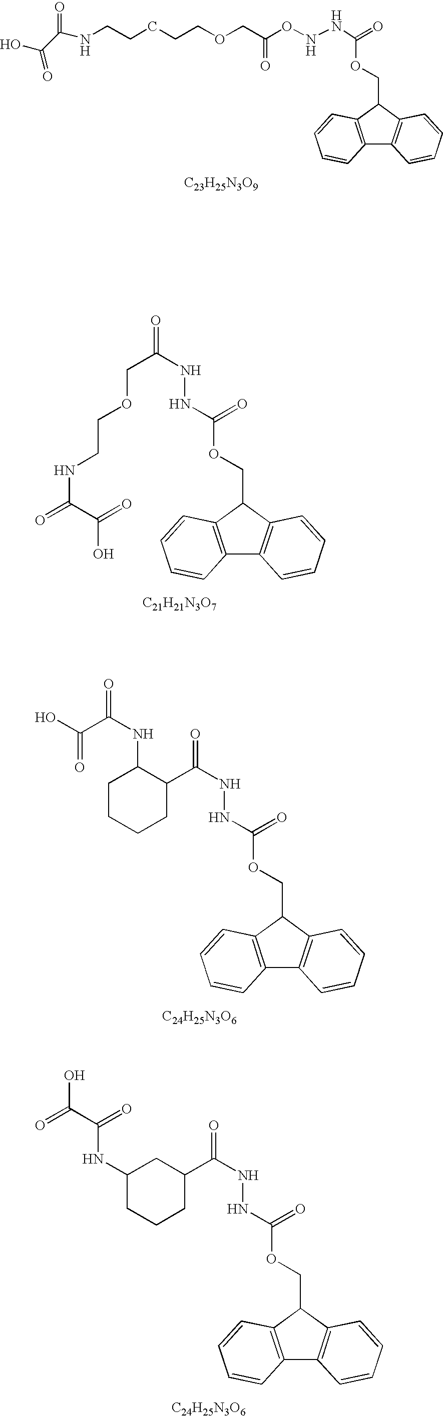

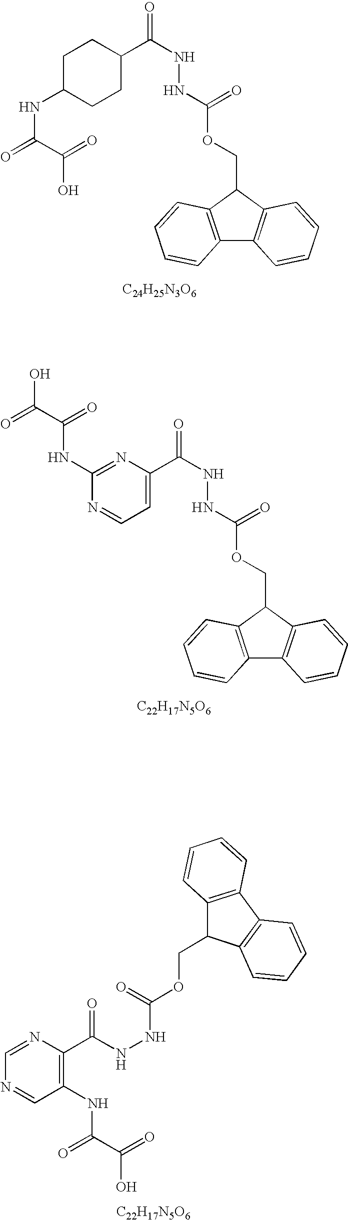

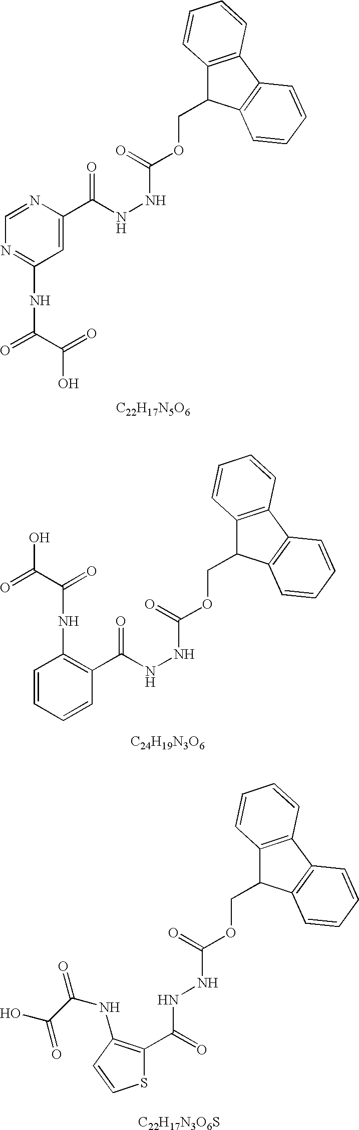

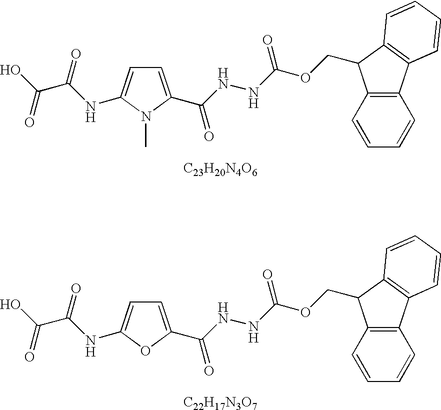

50. A method of making a compound of claim 25, comprising:
contacting, in any order, a compound of Formula 7: 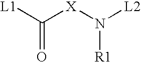
with a hydrazine or hydrazine derivative of Formula 8: 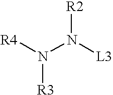
and an oxalic acid, ester or derivative of Formula 9: 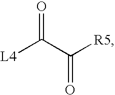
wherein L¹, L², L³ and L⁴ are independent arbitrary leaving groups or hydrogen.

51. The method of claim 50, wherein L4 is Cl, OH, an activated ester, or an anhydride.

52. The method of claim 51, wherein one of R³ or R⁴ is a protecting group.

53. The method of claim 52, wherein one of R³ or R⁴ is Fmoc.

54. A peptide or protein mimetic comprising a compound of claim 1 as a molecular part and a natural or unnatural amino acid or an additional amino acid mimetic.

55. The peptide or protein mimetic of claim 54, wherein the molecular part and a natural or unnatural amino acid or an additional amino acid mimetic are bound by an amide bond.

56. The peptide or protein mimetic of claim 54, further defined as comprising at least 2 natural or unnatural amino acids or additional amino acid mimetic in addition to the molecular part.

57. The peptide or protein mimetic of claim 56, further defined as comprising at least 3 natural or unnatural amino acids or additional amino acid mimetic in addition to the molecular part.

58. The peptide or protein mimetic of claim 57, further defined as comprising at least 5 natural or unnatural amino acids or additional amino acid mimetic in addition to the molecular part.

59. The peptide or protein mimetic of claim 58, further defined as comprising at least 10 natural or unnatural amino acids or additional amino acid mimetic in addition to the molecular part.

60. The peptide or protein mimetic of claim 59, further defined as comprising at least 20 natural or unnatural amino acids or additional amino acid mimetic in addition to the molecular part.

61. The peptide or protein mimetic of claim 54, further defined as a mimic of three dimensional structure of an alpha-helix, beta-sheet, or turn motif.

62. A method for the synthesis of a peptide mimetic of claim 54, comprising using a compound of the general Formula 1, wherein a reaction target comprising an amine is contacted with the compound of Formula 1, and R³ or R⁴ is an amide with a protecting group.

63. The method of claim 62, wherein R³ or R⁴ is Fmoc.

64. The method of claim 62, further comprising removing the R³ or R⁴ protecting group.

65. The method of claim 64, wherein an amino acid or amino acid mimetic is contacted with the compound of Formula 1.

66. The method of claim 64, wherein an amino acid or amino acid mimetic comprises a protected amino group.

67. The method of claim 64, wherein the amino acid or amino acid mimetic is contacted with the compound of Formula 1 after removing the R³ or R⁴ protecting group.

68. The method of claim 62, wherein the reaction target is solid.

69. The method of claim 62, wherein the reaction target is a solid resin.

70. A peptide, protein or protein fragment comprising a compound of the general Formula 1 as a covalently bound insert or replacement at any position of the protein amino acid sequence.

71. A method for the manufacture of a peptide, protein or protein fragment of claim 70, comprising ligating a compound of Formula 1 to a peptide, protein or protein fragment.

72. The method of claim 71, further comprising ligating a further amino acid, peptide, protein or protein fragment to the compound of Formula 1.

73. A compound comprising a compound of claim 1 as a spacer moiety.

74. A combinatorial library comprising a compound of claim 1.
